SEQUENCE LISTING (4 sequences)

SEQ ID NO: 1 - protein, 926 aa, Homo sapiens ETAA1
MSRRRKHDDSPSPKKTPHKTVAAEECGSVVEPGRRRLRSARGSWPCGAREGPPGPVRQREQPPTAALCSKSNPEERYETPKRALKMDSLSSSFSSPNDPDGQNDIFWDQNSPLTKQLGKGRKKQIYTTDSDEISHIVNRIAPQDEKPTTNSMLDMWIGETAIPCTPSVAKGKSRAKISCTKLKTQSQEEELMKLAKQFDKNMEELDVIQEQNKRNYDFTQMISETEILSNYKDNIQMWSLHNIVPEIDNATKKPIKGNTKISVANNQNSSQKPFDQIAEAAFNAIFDGSTQKCSGQLSQELPEAFWSTSNTTFVKTNALKEEKIITNETLVIEKLSNKTPRSLSSQVDTPIMTKSCVTSCTKEPETSNKYIDAFTTSDFEDDWENLLGSEPFAMQNIDMPELFPSKTAHVTDQKEICTFNSKTVKNTSRANTSPDARLGDSKVLQDLSSKTYDRELIDAEYRFSPNSNKSNKLSTGNKMKFENSSNKIVIQDEIQNCIVTSNLTKIKEDILTNSTEASERKSALNTRYSNEQKNKCILNQSIKAPVNTDLFGSANLGSKTSVSNPNQTSASKVGSFFDDWNDPSFANEIIKACHQLDNTWEADDVDDDLLYQACDDIERLTQQQDIRKDSKTSESICEINNNSEHGAKLTQQQDIRKDSKTSESICEINNNSEHGAKNMFAISKQGSNLVQSKHLNPGSISVQTSLTNSSQIDKPMKMEKGEMYGNSPRFLGATNLTMYSKISNCQINNLHVSYTNTDVPIQVNSSKLVLPGSSSLNVTSDHMNTEITTYKKKLSTNQPCHKTVTDEAQSNLNTTVGFSKFTFTRMKNSQILSQFNQNCITGSMSDTKITQGVEKKKGVNPLLEEAVGQQSLVKLSESLKQSSKEEEEKNRKCSPEEIQRKRQEALVRRMAKARASSVNAAPTSFL

SEQ ID NO: 2 - protein, 175 aa, Homo sapiens ETAA1-ADD
VRQREQPPTAALCSKSNPEERYETPKRALKMETDSLSSSFSSPNDPDGQNDIFWDQNSPLTKQLGKGRKKQIYTTDSDEISHIVNRIAPQDEKPTTNSMETLDMETWIGETAIPCTPSVAKGKSRAKISCTKLKTQSQEEELMETKLAKQFDKNMETEELDVIQEQNKRNYDFTQ

SEQ ID NO: 3 - DNA, 576 bp, Homo sapiens ETAA1
gtgcggcagcgagagcagcctccgaccgccgccctgtgcagtaaaagtaaccccgaggaaaggtatgaaacaccaaagagagcgctgaaaatggactcactgtcatcttccttcagttctcctaatgatccagatggacagaatgatatcttttgggatcagaattctccattgacaaagcagttaggtaaaggaagaaaaaaacagatttacaccacagatagtgatgagatttcacatattgttaatcgtattgctcctcaggatgaaaaaccaacaacaaattctatgctggacatgtggattggtgaaactgctattccttgtactcccagtgtagcaaaaggaaaatcaagagcaaaaatcagctgcacaaagttaaaaacacaaagtcaagaagaagaacttatgaaactggctaaacaatttgataaaaatatggaagagctagatgtgattcaagagcaaaacaagaggaattatgattttacccaggctcgagctgatccaaaaaagaagagaaaggtagatccaaaaaagaagagaaaggtagatccaaaaaagaagagaaaggta

SEQ ID NO: 4 - DNA, 495 bp, Homo sapiens ETAA1-ADD
gtgcggcagcgagagcagcctccgaccgccgccctgtgcagtaaaagtaaccccgaggaaaggtatgaaacaccaaagagagcgctgaaaatggactcactgtcatcttccttcagttctcctaatgatccagatggacagaatgatatcttttgggatcagaattctccattgacaaagcagttaggtaaaggaagaaaaaaacagatttacaccacagatagtgatgagatttcacatattgttaatcgtattgctcctcaggatgaaaaaccaacaacaaattctatgctggacatgtggattggtgaaactgctattccttgtactcccagtgtagcaaaaggaaaatcaagagcaaaaatcagctgcacaaagttaaaaacacaaagtcaagaagaagaacttatgaaactggctaaacaatttgataaaaatatggaagagctagatgtgattcaagagcaaaacaagaggaattatgattttacccag

(19) United States
(12) Patent Application Publication
(10) Pub. No.: US 2020/0131473 A1
(43) Pub. Date: Apr. 30, 2020

(54) METHOD OF GENERATING 2 CELL-LIKE STEM CELLS

(71) Applicant: IFOM FONDAZIONE ISTITUTO FIRC DI ONCOLOGIA MOLECOLARE, Milano (IT)

(72) Inventors: Vincenzo COSTANZO, Napoli (IT); Sina ATASHPAZGARGARI, Milano (IT); Sara SAMADI SHAMS, Milano (IT)

(21) Appl. No.: 16/494,249

(22) PCT Filed: Mar. 20, 2018
(86) PCT No.: PCT/EP2018/056986

§ 371 Date: Sep. 13, 2019

(30) Foreign Application Priority Data
Mar. 20, 2017 (EP) 17161888.7

(51) Int. Cl. C12N 5/073; C12N 15/86

(52) U.S. Cl. CPC C12N 5/0603; C12N 15/86; C12N 2740/15043; C12N 2500/02; C12N 2501/999; C12N 2506/02

(57) ABSTRACT

The present invention relates to 2 cell-like stem cells and particularly, although not exclusively, the generation of such cells from pluripotent stem cells.

### RELATED APPLICATIONS

This application claims priority from EP 17161888.7 filed 20 Mar. 2017, the contents and elements of which are herein incorporated by reference for all purposes.

### FIELD OF THE INVENTION

The present invention relates to 2 cell-like stem cells and particularly, although not exclusively, the generation of such cells from pluripotent stem cells.

### BACKGROUND TO THE INVENTION

Early cellular events after fertilization are of immense importance for the better understanding of the embryonic development during which extensive reprogramming of the genome is followed by transition from totipotent to pluripotent state. The zygote and its daughter cells (2-cell-stage blastomeres) are the only known totipotent cells as they are capable of generating an entire organism while inner cell mass (ICM)-derived embryonic stem cells (ESC) are regarded as pluripotent because they have the ability to contribute to embryonic but not extra-embryonic tissues.

Recent studies have identified 2C-like cells as a rare transient cell population (˜1-5%) within mouse embryonic (ESC) and induced pluripotent stem cell (iPSC) cultures that express high levels of transcripts also found in totipotent two-cell (2C) embryos. Notably, nearly all PSCs cycle in and out of this metastable state, at least once during nine passages that is accompanied by the transient transcriptional activation of endogenous retroviruses (ERVs), in particular MuERV-L. MuERV-L is highly abundant mRNA at the 2-cell stage of development but it undergoes repression through various epigenetic silencing mechanisms at the blastocyst stage. Recent data indicates that MuERV-L regulates the unique transcriptional signature of both 2C-like cells and 2C embryos through various mechanisms including: the activation of enhancers, introducing alternative promoters and generation of chimeric transcripts for early embryonic mouse genes. Interestingly, the remarkable genomic stability of mouse pluripotent stem cells has been recently linked to the transient bursts of Zscan4 (a well-known 2C marker) expression during this metastable state. Zscan4 knockdown in PSCs shortens telomeres, increases karyotype abnormalities and spontaneous sister chromatid exchanges that subsequently leads to crisis by passage eight. These results overall suggest that transition to 2C-like state in PSC culture could be beneficial to maintain the genomic stability of these cells.

Although the extended developmental potential of 2C-like cells in generating embryonic plus extra-embryonic tissues as well as their higher reprogrammability through SCNT assay has been explored by several groups the molecular mechanism underlying the reactivation of 2C-like transcriptional signature is not well investigated.

### SUMMARY OF THE INVENTION

The present invention concerns the use of replication stress (RS) response and DNA damage signalling pathways to regulate the transition of pluripotent stem cells into the 2C-like state. Increasing the proportion of 2C-like cells, such as transiently increasing the number of 2C-like cells, in a population of cells may be useful for increasing the genomic stability of that population. This may allow a population of stem cells to maintain pluripotency for longer. It may allow a larger number of passages of the stem cell culture, whilst maintaining the developmental potential or pluripotency of the cell population. Providing 2C like cells may allow the generation of extra-embryonic tissues.

Disclosed herein are methods for inducing the expression of one or more 2 cell-like genes in a pluripotent stem cell, the method comprising activating replication stress or inducing replication stress in a pluripotent stem cell. The method may involve exposing the pluripotent cells to one or more DNA damage inducing agents, such as culturing the pluripotent stem cell in the presence of one or more agents selected from Aphidicolin, ATR (ATM and Rad3-related kinase), hydroxyurea or high O₂ or exposing the pluripotent stem cell to UV radiation or ionising radiation. The method may involve the overexpression of, or activation of signalling associated with ETAA1 (Ewing's tumor-associated antigen 1). The method may involve overexpression of, or activation of, signalling associated with ATR or CHK1. The method may involve the administration of ATR or CHK1 protein.

In some cases, the treatment induces totipotency in some or all cells. In some cases, cells develop an increased developmental potential as a result of the method. In some cases, the cells express one or more 2C-like genes.

Also disclosed are methods for inducing the expression of one or more 2 cell-like genes in a pluripotent stem cell, the method comprising activating the ATR pathway in the pluripotent stem cell.

Also described are methods for the generation of extra-embryonic tissue. These methods may involve the activation of replication stress response or induction of replication stress in an embryo or embryonic stem cell, preferably an embryo.

Methods disclosed herein may involve overexpressing ETAA1 or an ATR binding fragment or ETAA1 in a pluripotent stem cell. In some methods, this involves infecting a pluripotent stem cell with a lentivirus that expresses ETAA1 or an ATR binding fragment thereof. The method may involve the injection of a vector or lentivirus comprising nucleic acid encoding a polypeptide having at least 80%, at least 85%, at least 90%, at least 95%, at least 98%, at least 99% or 100% identity to SEQ ID NO: 1.

In some cases, the method involves overexpressing a polypeptide having at least 80%, at least 85%, at least 90%, at least 95%, at least 98%, at least 99% or 100% identity to SEQ ID NO: 1 in a cell. The polypeptide preferably binds to ATR.

Also disclosed herein is vector such as an expression vector comprising nucleic acid encoding a polypeptide having at least 80%, at least 85%, at least 90%, at least 95%, at least 98%, at least 99% or 100% identity to SEQ ID NO: 1.

Methods disclosed herein result in the expression of one or more 2 cell-like genes in a pluripotent stem cell, wherein the 2 cell-like genes may be selected from the group consisting or comprising of Eif1a-like genes (Gm5662, Gm2022, Gm4027, Gm2016, and Gm8300), MERVL, MT2_Mm, Dux, Gm4981, Zfp352, Zfp750, Tdpoz genes (Tdpozl and Tdpoz3), and Tmem92, Zscan4 genes (Zscan4a-Zscan4d), Dux, Tcstv3, GM12794, γH2AX, P-CHK1, GM4340, Gm12794, Afp352, Sfp750, Tdpoz, Eif1a and Tmem19.

Expression of 2 cell-like (2C-like) genes in a pluripotent stem cell may result in the cell becoming totipotent, or substantially totipotent. The resultant cell may be capable of differentiating into embryonic or extra-embryonic tissues, and the methods disclosed herein may involve a step of differentiating the cell into an embryonic or extra-embryonic tissue cell.

Pluripotent stem cells useful in the methods disclosed herein include induced pluripotent stem cells or embryonic stem cells. The cells may be human, such as human embryonic stem cells. In some cases disclosed herein, the embryonic stem cell is a non-human embryonic stem cell. Disclosed herein are pluripotent stem cells containing a plasmid encoding ETAA1 or an ATR binding fragment thereof.

Also disclosed herein are 2 cell-like stem cells prepared by the methods disclosed herein, and populations of cells containing a high proportion of 2 cell-like stem cells. These cells express one or more of Eif1a-like genes (Gm5662, Gm2022, Gm4027, Gm2016, and Gm8300), MERVL, MT2_Mm, Dux, Gm4981, Zfp352, Zfp750, Tdpoz genes (Tdpozl and Tdpoz3), and Tmem92, Zscan4 genes (Zscan4a-Zscan4d) Dux, Tcstv3, GM12794, γH2AX, P-CHK1, GM4340, Gm12794, Afp352, Sfp750, Tdpoz, Eif1a and Tmem19. The methods may induce the transient expression of one or more of these markers. In some cases, the methods disclosed herein may induce expression, such as transient expression, of Zscan4 genes.

In some cases, the methods disclosed herein may induce expression, such as transient expression, of Zscan4, Tcstv3, GM12794 and Eif1a 2. In some cases, the methods disclosed herein lead to the expression of γH2AX and p-CHK1⁺ in the cells. Methods disclosed herein result in a high proportion of 2C-like cells in a pluripotent stem cell population. Accordingly, also provided herein is a population of pluripotent stem cells, such as a culture of pluripotent stem cells wherein at least 6%, 7%, 8%, 9% or 10% of the stem cells express one or more 2C-like genes. In some cases, the cells are induced to express one or more damage associated genes, particularly ATR signalling genes such as γH2AX, P-CHK1. Also disclosed herein are induced totipotent stem cells, or 2C-like stem cells produced by the methods disclosed herein. The cells may be genetically engineered to express ETAA1 or an ATR binding fragment thereof.

Also disclosed herein is a composition comprising pluripotent stem cells and a DNA damage agent, or a composition comprising pluripotent stem cells that overexpress ETAA1 or an ATR binding fragment thereof. In some cases, a composition comprising a pluripotent stem cell comprising a heterogenous nucleic acid encoding ETAA1 or an ATR binding fragment thereof is provided. In some aspects, 2C-like cells are isolated from other cells, such as pluripotent stem cells in the culture. For example, cells may be isolated from pluripotent cells that do not express one or more 2C-like genes. The cells may be isolated on the basis of the expression of one or more 2C-like genes, such as isolated based on their expression of Zscan4. In these aspects, a population containing substantially all 2C-like cells is obtained. Such a population may have a reduced proportion of pluripotent stem cells that do not express 2C-like genes, or substantially no pluripotent stem cells that do not express 2C-like genes.

In one aspect, the disclosure provides culture medium comprising a DNA damage inducing agent, such as Aphidicolin, hydroxyurea or high O₂. In another aspect, the invention provides the use of such a DNA damage inducing agent in the culture of stem cells.

Also disclosed herein is stem cell culture medium comprising a DNA damage inducing agent. The culture medium may contain a DNA damage agent selected from Aphidicolin, hydroxyurea or high O₂.

### DESCRIPTION

The invention includes the combination of the aspects and preferred features described except where such a combination is clearly impermissible or expressly avoided.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

The zygote and its daughter cells (2C stage blastomeres) are the only known totipotent cells as they are capable of generating an entire organism while inner cell mass-derived embryonic stem cells (ESC) are regarded as pluripotent because they have the ability to contribute to embryonic but not extra-embryonic tissues 1-3. Recent studies have identified 2C-like cells as a rare transient cell population (˜1-5%) within pluripotent stem cell (PSC) cultures that exhibit transcriptional and functional features of totipotent 2C-embryos 2,4-6.

Although the extended developmental potential of 2C-like cells in generating embryonic plus extra-embryonic tissues as well as their higher reprogrammability through SCNT assay has been explored by several groups 1, 2, 6, 7, the molecular mechanism and the physiological relevance underlying the transition to of 2C-like cells are not well understood.

Here, we show that ATR activation in embryonic stem cells (ESCs) and mouse embryos triggers transition to 2C-like cell. We provide several lines of evidence that ATR and CHK1-mediated response to RS triggers transition to 2C-like state in ESCs and mouse embryos. This transition is abolished or hampered in ESCs derived from ATR-defective Seckel and CHK1 haploinsufficinet mice models and following inhibition of ATR or CHK1. More importantly, 2C event is stimulated in the absence of replication stress through ETAA1-mediated direct ATR activation. Significantly, we show that ETAA1-mediated direct ATR activation is sufficient to potently trigger the activation of 2C-like cells in the absence of RS. Mechanistically, ATR-induced transition to 2C-like state is mediated by activation of the Dux gene, which shapes the transcriptional signature of 2C-like cells and totipotent 2C-stage embryos in placental mammals⁸⁻¹⁰. In addition, ATR activation is unable to enhance the formation of placental giant trophoblast-like cells (TGCs) in both DUX knockout (KO) and ATR-deficient Seckel ESCs. Strikingly, ATR-activated ESCs exhibit expanded cell fate potential in vivo, as shown by their ability to contribute to both inner cell mass and extra-embryonic compartments.

In ESCs, ATR activation induces a specific transcriptional program characterized by: 1) a prominent expression of early embryo-specific and genome caretaker genes such as Zscan4 and Tcstv1/3, known to improve genomic stability of ESCs^{5,11,12}, and 2) the induction of the trophectoderm directed differentiation program that minimizes contribution of ESCs with active checkpoints to inner cell mass, by directing them, instead, to extra-embryonic compartment.

Overall, we uncovered a fundamental ATR-CHK1-mediated response that ensures genomic integrity during mammalian development, which is achieved by potential increase in the repair capacity of ESCs and through reprogramming ESCs to a more primitive and developmentally potent stage.

Stem Cells

As used in this document, the term “stem cell” refers to a cell that on division faces two developmental options: the daughter cells can be identical to the original cell (self-renewal) or they may be the progenitors of more specialised cell types (differentiation).

The stem cell is therefore capable of adopting one or other pathway (a further pathway exists in which one of each cell type can be formed). Stem cells are therefore cells which are not terminally differentiated and are able to produce cells of other types.

In general, reference herein to cells (plural) may include the singular (stem cell), and vice versa. In particular, methods of culturing and differentiating stem cells may include single cell and aggregate culturing techniques. In the present invention stem cell cultures may be of aggregates or single cells.

Stem cells can be described in terms of the range of cell types into which they are able to differentiate, as discussed below. The stem cells obtained or produced by the methods of the present invention are preferably at least pluripotent. Optionally, they are multipotent.

“Totipotent” stem cells refers to a cell which has the potential to become any cell type in the adult body, or any cell of the extraembryonic membranes (e.g., placenta). Thus, normally, the only totipotent cells are the fertilized egg and the first 4 or so cells produced by its cleavage.

“Pluripotent” stem cells are true stem cells, with the potential to make any differentiated cell in the body. However, they cannot contribute to make the extraembryonic membranes which are derived from the trophoblast. Embryonic Stem (ES) cells are examples of pluripotent stem cells, and may be isolated from the inner cell mass (ICM) of the blastocyst, which is the stage of embryonic development when implantation occurs.

The stem cells may be obtained or produced by the methods of the present invention, and are normally created from non-pluripotent cells such as somatic cells, and may therefore be referred to as “induced pluripotent stem cells”.

Methods of characterising stem cells are known in the art, and include the use of standard assay methods such as clonal assay, flow cytometry, long-term culture and molecular biological techniques e.g. PCR, RT-PCR and Southern blotting.

In addition to morphological differences, human and murine pluripotent stem cells differ in their expression of a number of cell surface antigens (stem cell markers). Markers for stem cells and methods of their detection are described elsewhere in this document (under “Maintenance of Stem Cell Characteristics”).

The present invention includes techniques for the generation of totipotent cells from pluripotent cells. The “stem cells” generated by the methods of the present invention have not been obtained by a method that causes the destruction of an embryo. In particular, the pluripotent cells of the present invention have been obtained by a method that does not cause the destruction of a human or mammalian embryo. As such, methods of the invention may be performed using cells that have not been prepared exclusively by a method which necessarily involves the destruction of human embryos from which those cells may be derived. Indeed, cells obtained from embryos are not required to perform methods according to the present invention. This optional limitation is specifically intended to take account of Decision G0002/06 of 25 Nov. 2008 of the Enlarged Board of Appeal of the European Patent Office.

### Sources of Pluripotent Cells

Several methods have now been provided for the isolation of pluripotent stem cells that do not lead to the destruction of an embryo, e.g. by transforming (inducing) adult somatic cells or germ cells. These methods include:

1. Reprogramming by nuclear transfer. This technique involves the transfer of a nucleus from a somatic cell into an oocyte or zygote. In some situations this may lead to the creation of an animal-human hybrid cell. For example, cells may be created by the fusion of a human somatic cell with an animal oocyte or zygote or fusion of a human oocyte or zygote with an animal somatic cell.
2. Reprogramming by fusion with embryonic stem cells. This technique involves the fusion of a somatic cell with an embryonic stem cell. This technique may also lead to the creation of animal-human hybrid cells, as in 1 above.
3. Spontaneous re-programming by culture. This technique involves the generation of pluripotent cells from non-pluripotent cells after long term culture. For example, pluripotent embryonic germ (EG) cells have been generated by long-term culture of primordial germ cells (PGC) (Matsui et al., Derivation of pluripotential embryonic stem cells from murine primordial germ cells in culture. Cell 70, 841-847, 1992, incorporated herein by reference). The development of pluripotent stem cells after prolonged culture of bone marrow-derived cells has also been reported (Jiang et al., Pluripotency of mesenchymal stem cells derived from adult marrow. Nature 418, 41-49, 2002, incorporated herein by reference). They designated these cells multipotent adult progenitor cells (MAPCs). Shinohara et al also demonstrated that pluripotent stem cells can be generated during the course of culture of germline stem (GS) cells from neonate mouse testes, which they designated multipotent germline stem (mGS) cells (Kanatsu-Shinohara et al., Generation of pluripotent stem cells from neonatal mouse testis. Cell 119, 1001-1012, 2004).
4. Reprogramming by defined factors. For example, the generation of iPS cells by the retrovirus-mediated introduction of transcription factors (such as Oct-3/4, Sox2, c-Myc, and KLF4) into mouse embryonic or adult fibroblasts, e.g. as described above. Kaji et al (Virus-free induction of pluripotency and subsequent excision of reprogramming factors. Nature. Online publication 1 Mar. 2009) also describe the non-viral transfection of a single multiprotein expression vector, which comprises the coding sequences of c-Myc, Klf4, Oct4 and Sox2 linked with 2A peptides, that can reprogram both mouse and human fibroblasts. iPS cells produced with this non-viral vector show robust expression of pluripotency markers, indicating a reprogrammed state confirmed functionally by in vitro differentiation assays and formation of adult chimaeric mice. They succeeded in establishing reprogrammed human cell lines from embryonic fibroblasts with robust expression of pluripotency markers.

Methods 1-4 are described and discussed by Shinya Yamanaka in Strategies and New Developments in the Generation of Patient-Specific Pluripotent Stem Cells (Cell Stem Cell 1, July 2007 ^{a}2007 Elsevier Inc), incorporated herein by reference.

5. Derivation of hESC lines from single blastomeres or biopsied blastomeres. See Klimanskaya I, Chung Y, Becker S, Lu S J, Lanza R. Human embryonic stem cell lines derived from single blastomeres. Nature 2006; 444:512, Lei et al Xeno-free derivation and culture of human embryonic stem cells: current status, problems and challenges. Cell Research (2007) 17:682-688, Chung Y, Klimanskaya I, Becker S, et al. Embryonic and extraembryonic stem cell lines derived from single mouse blastomeres. Nature. 2006; 439:216-219. Klimanskaya I, Chung Y, Becker S, et al. Human embryonic stem cell lines derived from single blastomeres. Nature. 2006; 444:481-485. Chung Y, Klimanskaya I, Becker S, et al. Human embryonic stem cell lines generated without embryo destruction. Cell Stem Cell. 2008; 2:113-117 and Dusko Ilic et al (Derivation of human embryonic stem cell lines from biopsied blastomeres on human feeders with a minimal exposure to xenomaterials. Stem Cells And Development—paper in pre-publication), all incorporated herein by reference.
6. hESC lines obtained from arrested embryos which stopped cleavage and failed to develop to morula and blastocysts in vitro. See Zhang X, Stojkovic P, Przyborski S, et al. Derivation of human embryonic stem cells from developing and arrested embryos. Stem Cells 2006; 24:2669-2676 and Lei et al Xeno-free derivation and culture of human embryonic stem cells: current status, problems and challenges. Cell Research (2007) 17:682-688, both incorporated herein by reference.
7. Parthogenesis (or Parthenogenesis). This technique involves chemical or electrical stimulation of an unfertilised egg so as to cause it to develop into a blastomere from which embryonic stem cells may be derived. For example, see Lin et al. Multilineage potential of homozygous stem cells derived from metaphase II oocytes. Stem Cells. 2003; 21(2):152-61 who employed the chemical activation of nonfertilized metaphase II oocytes to produce stem cells.
8. Stem cells of fetal origin. These cells lie between embryonic and adult stem cells in terms of potentiality and may be used to derive pluripotent or multipotent cells. Human umbilical-cord-derived fetal mesenchymal stem cells (UC fMSCs) expressing markers of pluripotency (including Nanog, Oct-4, Sox-2, Rex-1, SSEA-3, SSEA-4, Tra-1-60, and Tra-1-81, minimal evidence of senescence as shown by 3-galactosidase staining, and the consistent expression of telomerase activity) have been successfully derived by Chris H. Jo et al (Fetal mesenchymal stem cells derived from human umbilical cord sustain primitive characteristics during extensive expansion. Cell Tissue Res (2008) 334:423-433, incorporated herein by reference). Winston Costa Pereira et al (Reproducible methodology for the isolation of mesenchymal stem cells from human umbilical cord and its potential for cardiomyocyte generation J Tissue Eng Regen Med 2008; 2: 394-399, incorporated herein by reference) isolated a pure population of mesenchymal stem cells from Wharton's jelly of the human umbilical cord. Mesenchymal stem cells derived from Wharton's jelly are also reviewed in Troyer & Weiss (Concise Review: Wharton's Jelly-Derived Cells Are a primitive Stromal Cell Population. Stem Cells 2008:26:591-599). Kim et al (Ex vivo characteristics of human amniotic membrane-derived stem cells. Cloning Stem Cells 2007 Winter; 9(4):581-94, incorporated herein by reference) succeeded in isolating human amniotic membrane-derived mesenchymal cells from human amniotic membranes. Umbilical cord is a tissue that is normally discarded and stem cells derived from this tissue have tended not to attract moral or ethical objection.
9. Chung et al. [(2008) Human Embryonic Stem Cell Lines Generated without Embryo Destruction. Cell Stem Cell. 2(2) 113-117. Epub 2008 Jan. 10] describes the generation of human embryonic stem cell lines with the destruction of an embryo. The method described by Chung et al (item 9 above) also permits obtaining of human embryonic stem cells by a method that does not cause the destruction of a human embryo.
10. WO 2003/046141 (5 Jun. 2003) described methods of parthenogenetic activation of human embryos, and the generation of embryonic stem cells therefrom. Such methods permit obtaining of human embryonic stem cells by a method that does not cause the destruction of a human embryo.

Induced pluripotent stem cells have the advantage that they can be obtained by a method that does not cause the destruction of an embryo, more particularly by a method that does not cause the destruction of a human or mammalian embryo. In certain methods disclosed herein, the pluripotent cells are induced pluripotent stem cells. Methods of generating induced pluripotent stem cells are well known in the art, and include induced pluripotent cells generated through the expression of one or more of the “Yamanaka factors” (Oct3/4, Sox2, Klf4, c-Myc) in a somatic cell.

As such, aspects of the invention may be performed or put into practice by using cells that have not been prepared exclusively by a method which necessarily involves the destruction of human or animal embryos from which those cells may be derived. This optional limitation is specifically intended to take account of Decision G0002/06 of 25 Nov. 2008 of the Enlarged Board of Appeal of the European Patent Office.

Pluripotent stem cells useful in the methods disclosed herein can be from any type of animal. In some preferred embodiments the cell is from a primate. The cells may be from non-human cells, e.g. rabbit, guinea pig, rat, mouse or other rodent (including cells from any animal in the order Rodentia), cat, dog, pig, sheep, goat, cattle, horse, non-human primate or other non-human vertebrate organism; and/or non-human mammalian cells. Preferably, the cells are human cells.

### Culture of Stem Cells

Any suitable method of culturing cells, including induced pluripotent stem cells, may be used in the methods and compositions described here. Methods may involve the culture medium that includes DNA damage inducing agents or agents that activate ATR.

Any suitable container may be used to propagate cells according to the methods and compositions described here. Suitable containers include those described in US Patent Publication US2007/0264713.

Containers may include bioreactors and spinners, for example. A “bioreactor”, as the term is used in this document, is a container suitable for the cultivation of eukaryotic cells, for example animal cells or mammalian cells, such as in a large scale. A typical cultivation volume of a regulated bioreactor is between 20 ml and 500 ml.

The bioreactor may comprise a regulated bioreactor, in which one or more conditions may be controlled or monitored, for example, oxygen partial pressure. Devices for measuring and regulating these conditions are known in the art. For example, oxygen electrodes may be used for oxygen partial pressure. The oxygen partial pressure can be regulated via the amount and the composition of the selected gas mixture (e.g., air or a mixture of air and/or oxygen and/or nitrogen and/or carbon dioxide). Suitable devices for measuring and regulating the oxygen partial pressure are described by Bailey, J E. (Bailey, J E., Biochemical Engineering Fundamentals, second edition, McGraw-Hill, Inc. ISBN 0-07-003212-2 Higher Education, (1986)) or Jackson A T. Jackson A T., Verfahrenstechnik in der Biotechnologie, Springer, ISBN 3540561900 (1993)).

Other suitable containers include spinners. Spinners are regulated or unregulated bioreactors, which can be agitated using various agitator mechanisms, such as glass ball agitators, impeller agitators, and other suitable agitators. The cultivation volume of a spinner is typically between 20 ml and 500 ml. Roller bottles are round cell culture flasks made of plastic or glass having a culture area of between 400 and 2000 cm². The cells are cultivated along the entire inner surface of these flasks; the cells are coated with culture medium accomplished by a “rolling” motion, i.e. rotating the bottles about their own individual axis.

Alternatively, culture may be static, i.e. where active agitation of the culture/culture media is not employed. By reducing agitation of the culture aggregates of cells may be allowed to form. Whilst some agitation may be employed to encourage distribution and flow of the culture media over the cultured cells this may be applied so as not to substantially disrupt aggregate formation. For example, a low rpm agitation, e.g. less than 30 rpm or less than 20 rpm, may be employed.

### Co-Culture and Feeders

Culture methods for stem cells may comprise culturing cells in the presence or absence of co-culture. The term “co-culture” refers to a mixture of two or more different kinds of cells that are grown together, for example, stromal feeder cells. The two or more different kinds of cells may be grown on the same surfaces, such as particles or cell container surfaces, or on different surfaces. The different kinds of cells may be grown on different particles.

Feeder cells, as the term is used in this document, may mean cells which are used for or required for cultivation of cells of a different type. In the context of stem cell culture, feeder cells have the function of securing the survival, proliferation, and maintenance of cell pluripotency. Cell pluripotency may be achieved by directly co-cultivating the feeder cells. Alternatively, or in addition, the feeder cells may be cultured in a medium to condition it. The conditioned medium may be used to culture the stem cells.

By way of example, the inner surface of the container such as a culture dish may be coated with a feeder layer of mouse embryonic skin cells that have been treated so they will not divide. The feeder cells release nutrients into the culture medium which are required for pluripotent cell growth. The stem cells growing on particles may therefore be grown in such coated containers.

Arrangements in which feeder cells are absent or not required are also possible. For example, the cells may be grown in medium conditioned by feeder cells or stem cells.

### Media and Feeder Cells

Media for isolating and propagating stem cells can have any of several different formulas, as long as the cells obtained have the desired characteristics, and can be propagated further.

Suitable sources are as follows: Dulbecco's modified Eagles medium (DMEM), Gibco #11965-092; Knockout Dulbecco's modified Eagles medium (KO DMEM), Gibco #10829-018; 200 mM L-glutamine, Gibco #15039-027; non-essential amino acid solution, Gibco 11140-050; beta-mercaptoethanol, Sigma # M7522; human recombinant basic fibroblast growth factor (bFGF), Gibco #13256-029. Exemplary serum-containing embryonic stem (ES) medium is made with 80% DMEM (typically KO DMEM), 20% defined fetal bovine serum (FBS) not heat inactivated, 0.1 mM non-essential amino acids, 1 mM L-glutamine, and 0.1 mM beta-mercaptoethanol. The medium is filtered and stored at 4 degrees C. for no longer than 2 weeks. Serum-free embryonic stem (ES) medium is made with 80% KO DMEM, 20% serum replacement, 0.1 mM non-essential amino acids, 1 mM L-glutamine, and 0.1 mM beta-mercaptoethanol. An effective serum replacement is Gibco #10828-028. The medium is filtered and stored at 4 degrees C. for no longer than 2 weeks. Just before use, human bFGF is added to a final concentration of 4 ng/mL (Bodnar et al., Geron Corp, International Patent Publication WO 99/20741).

The media may comprise Knockout DMEM media (Invitrogen-Gibco, Grand Island, N.Y.), supplemented with 10% serum replacement media (Invitrogen—Gibco, Grand Island, N.Y.), 5 ng/ml FGF2 (Invitrogen-Gibco, Grand Island, N.Y.) and 5 ng/ml PDGF AB (Peprotech, Rocky Hill, N.J.).

Feeder cells (where used) may be propagated in mEF medium, containing 90% DMEM (Gibco #11965-092), 10% FBS (Hyclone #30071-03), and 2 mM glutamine. mEFs are propagated in T150 flasks (Corning #430825), splitting the cells 1:2 every other day with trypsin, keeping the cells subconfluent. To prepare the feeder cell layer, cells are irradiated at a dose to inhibit proliferation but permit synthesis of important factors that support human embryonic stem cells (about 4000 rads gamma irradiation). Six-well culture plates (such as Falcon #304) are coated by incubation at 37 degrees C. with 1 mL 0.5% gelatin per well overnight, and plated with 375,000 irradiated mEFs per well. Feeder cell layers are typically used 5 h to 4 days after plating. The medium is replaced with fresh human embryonic stem (hES) medium just before seeding pluripotent stem (pPS) cells.

Conditions for culturing other pluripotent cells are known, and can be optimized appropriately according to the cell type. Media and culture techniques for particular cell types referred to in the previous section are provided in the references cited.

### Serum Free Media

The methods and compositions described here may include culture of cells in a serum-free medium. In some cases, a composition comprising serum free medium and a DNA damage agent is provided.

The term “serum-free media” may comprise cell culture media which is free of serum proteins, e.g., fetal calf serum. Serum-free media are known in the art, and are described for example in U.S. Pat. Nos. 5,631,159 and 5,661,034. Serum-free media are commercially available from, for example, Gibco-BRL (Invitrogen).

The serum-free media may be protein free, in that it may lack proteins, hydrolysates, and components of unknown composition. The serum-free media may comprise chemically-defined media in which all components have a known chemical structure. Chemically-defined serum-free media is advantageous as it provides a completely defined system which eliminates variability, allows for improved reproducibility and more consistent performance, and decreases possibility of contamination by adventitious agents.

The serum-free media may comprise Knockout DMEM media (Invitrogen-Gibco, Grand Island, N.Y.).

The serum-free media may be supplemented with one or more components, such as serum replacement media, at a concentration of for example, 5%, 10%, 15%, etc. The serum-free media may be supplemented with 10% serum replacement media from Invitrogen—Gibco (Grand Island, N.Y.).

The serum-free medium in which the dissociated or disaggregated embryonic stem cells are cultured may comprise one or more growth factors. A number of growth factors are known in the art, including FGF2, IGF-2, Noggin, Activin A, TGF beta 1, HRG1 beta, LIF, S1P, PDGF, BAFF, April, SCF, Flt-3 ligand, Wnt3A and others. The growth factor(s) may be used at any suitable concentration such as between 1 pg/ml to 500 ng/ml.

Propagation with Passage The stem cells produced or obtained in accordance with the present invention may be maintained in cell culture. Such culture may comprise passaging, or splitting during culture. The methods may involve continuous or continual passage.

The term “passage” may generally refer to the process of taking an aliquot of a cell culture, dissociating the cells completely or partially, diluting and inoculating into medium.

The passaging may be repeated one or more times. The aliquot may comprise the whole or a portion of the cell culture. The cells of the aliquot may be completely, partially or not confluent. The passaging may comprise at least some of the following sequence of steps: aspiration, rinsing, trypsinization, incubation, dislodging, quenching, re-seeding and aliquoting. The protocol published by the Hedrick Lab, UC San Diego may be used (http://hedricklab.ucsd.edu/Protocol/COSCell.html).

The cells may be dissociated by any suitable means, such as mechanical or enzymatic means known in the art. The cells may be broken up by mechanical dissociation, for example using a cell scraper or pipette. The cells may be dissociated by sieving through a suitable sieve size, such as through 100 micron or 500 micron sieves. The cells may be split by enzymatic dissociation, for example by treatment with collagenase, Trypsin or TrypLE™ harvested. The dissociation may be complete or partial.

Cells in culture may be dissociated from the substrate or flask, and “split”, subcultured or passaged, by dilution into tissue culture medium and replating. The dilution may be of any suitable dilution. The cells in the cell culture may be split at any suitable ratio. For example, the cells may be split at a ratio of 1:2 or more, 1:3 or more, 1:4 or more or 1:5 or more. The cells may be split at a ratio of 1:6 or more, 1:7 or more, 1:8 or more, 1:9 or more or 1:10 or more. The split ratio may be 1:10 or more. It may be 1:11, 1:12, 1:13, 1:14, 1:15, 1:16, 1:17, 1:18, 1:19 or 1:20 or more. The split ratio may be 1:21, 1:22, 1:23, 1:24, 1:25 or 1:26 or more.

Thus, cells may be passaged for 1 passage or more. For example, stem cells may be passaged for 2, 3, 4, 5, 6, 7, 8, 9, 10, 11, 12, 13, 14, 15, 16, 17, 18, 19, 20, 21, 22, 23, 24, 25 passages or more. In principle, cells may be propagated indefinitely in culture.

In some methods disclosed herein, the expression of 2C-like genes is induced in an embryo. The embryo may be rodent embryo such as a murine embryo. It may be a primate embryo, such as a non-human primate embryo. In some cases, the embryo is not a human embryo. The method may involve the expression of ETAA1 or an ATR binding fragment thereof in the embryo, for example by the injection of an expression plasmid comprising nucleic acid encoding ETAA1 or an ATR binding fragment thereof into the embryo.

### Expression of Markers

Stem cells may also be characterized by expressed cell markers. The biological activity that is retained may comprise expression of one or more pluripotency markers.

Stage-specific embryonic antigens (SSEA) are characteristic of certain embryonic cell types. Antibodies for SSEA markers are available from the Developmental Studies Hybridoma Bank (Bethesda Md.). Other useful markers are detectable using antibodies designated Tra-1-60 and Tra-1-81 (Andrews et al., Cell Lines from Human Germ Cell Tumors, in E. J. Robertson, 1987, supra). Human embryonic stem cells are typically SSEA-1 negative and SSEA-4 positive. hEG cells are typically SSEA-1 positive. Differentiation of primate pluripotent stem cells (pPS) cells in vitro results in the loss of SSEA-4, Tra-1-60, and Tra-1-81 expression and increased expression of SSEA-1. pPS cells can also be characterized by the presence of alkaline phosphatase activity, which can be detected by fixing the cells with 4% paraformaldehyde, and then developing with Vector Red as a substrate, as described by the manufacturer (Vector Laboratories, Burlingame Calif.).

Embryonic stem cells are also typically telomerase positive and OCT-4 positive. Telomerase activity can be determined using TRAP activity assay (Kim et al., Science 266:2011, 1997), using a commercially available kit (TRAPeze® XK Telomerase Detection Kit, Cat. s7707; Intergen Co., Purchase N.Y.; or TeloTAGGG™ Telomerase PCR ELISA plus, Cat. 2,013,89; Roche Diagnostics, Indianapolis). hTERT expression can also be evaluated at the mRNA level by RT-PCR. The LightCycler TeloTAGGG™ hTERT quantification kit (Cat. 3,012,344; Roche Diagnostics) is available commercially for research purposes.

Any one or more of these pluripotency markers, including FOXD3, alkaline phosphatase, OCT-4, SSEA-4, and TRA-1-60 etc, may be expressed by pluripotent stem cells.

Methods disclosed herein result in the expression of one or more markers associated with 2C-like cells, or totipotent stem cells. Markers of 2C-like cells include Eif1a-like genes (Gm5662, Gm2022, Gm4027, Gm2016, and Gm8300), MERVL, MT2_Mm, Dux, Gm4981, Zfp352, Zfp750, Tdpoz genes (Tdpozl and Tdpoz3), and Tmem92, Zscan4 genes (Zscan4a-Zscan4d), Dux, Tcstv3, GM12794, γH2AX, P-CHK1, GM4340, Gm12794, Afp352, Sfp750, Tdpoz, Eif1a and Tmem19. Cells produced by the methods disclosed herein may express one, two, three, four, five, six, seven, eight, nine, ten, eleven, twelve, thirteen, fourteen, fifteen or sixteen of the markers. In some cases, the cells express one, two, three, four, five or several markers selected from the group consisting of Dux, Zscan4 gene family, Eif1a family, Tdpoz family and Tcstv family and muERVL elements.

Detection of markers may be achieved through any means known in the art, for example immunologically. Histochemical staining, flow cytometry (FACS), Western Blot, enzyme-linked immunoassay (ELISA), etc may be used.

Flow immunocytochemistry may be used to detect cell-surface markers. Immunohistochemistry (for example, of fixed cells or tissue sections) may be used for intracellular or cell-surface markers. Western blot analysis may be conducted on cellular extracts. Enzyme-linked immunoassay may be used for cellular extracts or products secreted into the medium.

For this purpose, antibodies to the 2C-like gene markers as available from commercial sources may be used.

The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods. Sequence data for the particular markers listed in this disclosure can be obtained from public databases such as GenBank See U.S. Pat. No. 5,843,780 for further details.

In some embodiments at least 5%, 6%, 7%, 8%, 9%, 10%, 11%, 12%, 13%, 14%, 15%, 16%, 17%, 18%, 19%, 20%, 21%, 22%, 23%, 24%, 25% or more of the cells in the culture express 2C-like genes.

### 2C-Like Cells

2C-like cells are a rare and transient cell population within pluripotent stem cell cultures that exhibit transcriptional and functional features of totipotent 2-cell-stage embryos.

2C-like cells may express high levels of transcripts found in totipotent two-cell embryos. 2C-like cells may express one or more of Eif1a-like genes (Gm5662, Gm2022, Gm4027, Gm2016, and Gm8300), MERVL, MT2_Mm, Dux, Gm4981, Zfp352, Zfp750, Tdpoz genes (Tdpozl and Tdpoz3), and Tmem92, Zscan4 genes (Zscan4a-Zscan4d) Dux, Tcstv3, GM12794, γH2AX, P-CHK1, GM4340, Gm12794, Afp352, Sfp750, Tdpoz, Eif1a and Tmem19. Accordingly, the term “2C-like cell” is used herein to refer to a cell that expresses one or more 2 cell-like gene.

2C-like cells may exhibit upregulation in one or more Eif1a-like genes (Gm5662, Gm2022, Gm4027, Gm2016, and Gm8300), MERVL, MT2_Mm, Dux, Gm4981, Zfp352, Zfp750, Tdpoz genes (Tdpozl and Tdpoz3), and Tmem92, Zscan4 genes (Zscan4a-Zscan4d) Dux, Tcstv3, GM12794, γH2AX, P-CHK1, GM4340, Gm12794, Afp352, Sfp750, Tdpoz, Eif1a and Tmem19. This may be the transcriptional signature of a 2C-like cell.

2C-like cells may have reduced expression of pluripotency markers at the protein but not transcriptional level. For example, 2C-like cells may have reduced expression of Nanog, Pou5f1 (Oct4) or Sox2 at the protein but not transcriptional level.

2C-like cells may be totipotent, or may have high reprogrammability in SCNT assay. The cells may not be truly totipotent, but instead may exhibit a complex cellular variation of totipotency.

2C-like cells may be capable of generating embryonic and extra-embryonic tissues, such as extraembryonic membrane, placental cells, yolk sac, allantois, amnion and chorion. The cells may be capable of differentiating into trophoblast cells.

### Replication Stress

Methods disclosed herein may involve inducing replication stress in cells. Replication stress is a stress occurring during DNA replication, typically resulting in a stalled replication fork. Replication stress can be due to DNA damage, excessive compacting of chromatin (preventing replisome access), over-expression of oncogenes or difficult-to-replicate genome structures. ATM and ATR (kinases recruited and activated by DNA damage) proteins mediate replication stress. Replication stress can lead to genome instability, cancer and aging.

In particular methods disclosed herein, replication stress is induced by DNA damage. DNA damage may be induced by a DNA damage inducing agent.

In the methods disclosed herein, replication stress may be induced by activation of the ATR dependent response. The ATR dependent response may be activated through the induction of DNA damage. The ATR dependent response may be activated through activation of the ATR kinase, e.g. by an ATR activating agent or ATR agonist. For example, though the binding of ETAA1 (Ewing's tumor-associated antigen 1) or a fragment thereof to ATR.

ATR (ATM and Rad3-related kinase) stabilizes and helps restart stalled replication forks, avoiding the generation of DNA damage and genome instability. Activation of the ATR dependent response may induce transcriptional alterations to produce 2C-like cells.

In some methods described herein, the induction of replication stress is indicated by the phosphorylation of H2AX and/or CHK1.

### DNA Damage Response

Cells have evolved a complex network of cellular pathways to mitigate the effects of genotoxic stress. The DNA Damage Response (DDR) is regulated by the kinases ATM and ATR. Both are activated in response to DNA damage and replication stress, but their effects are different. ATR is a serine/threonine kinase involved in sensing DNA damage and activating the DNA damage checkpoint (Chk1), leading to cell cycle arrest.

Methods disclosed herein involve the induction of replication stress or DNA damage response, in particular through the activation of ATR and/or Chk1.

### DNA Damage Inducing Agents

A DNA damage inducing agent is an agent capable of inducing DNA damage.

DNA damage inducing agents include aphidicolin, hydroxyurea, UV light, high O₂, and ionising radiation. In some cases, the DNA damage inducing agent is a Topoisomerase inhibitor, such as CPT, Topetecan, or Etoposide. In some cases, the DNA damage inducing agent is methyl methanesulfonate (MMS), Mitomycin C (MMC), Hydrogen peroxide (H₂O₂), or formaldehyde.

High O₂ conditions include around 15%, 16%, 17%, 18%, 19%, 20%, 21%, 22%, 23%, 24%, 25% O₂. Preferably, high O₂ conditions involve exposing the cells to about 20% O₂ or more.

Methods may involve exposing a pluripotent stem cell to an effective amount of DNA damage inducing agent. The method may involve culturing a pluripotent cell in a culture with a DNA damage inducing agent. Also disclosed herein is culture medium comprising an effective amount of DNA damage agent.

An effective amount of DNA damage may be an amount sufficient to initiate the expression of 2C-like genes in the cell. Preferably, the amount is not sufficient to induce apoptosis in the cell

In preferred aspects, Aphidicolin (APH) is used in an amount between 0.2 μM and 7 μM to induce the expression of 2C-like genes. For example, around 0.2 μM, 0.4 μM, 0.6 μM, 0.8 μM, 1 μM, 1.5 μM, 2 μM, 2.5 μM, 3 μM, 3.5 μM, 4 μM, 4.5 μM, 5 μM, 5.5 μM, 6 μM, 6.5 μM, or 7 μM may be used.

In preferred aspects Hydroxyurea (HU) is used in an amount between 0.1 mM and 4 mM. For example around 0.1 mM, 0.2 mM, 0.3 mM, 0.4 mM, 0.5 mM, 0.6 mM, 0.7 mM, 0.8 mM, 0.9 mM, 1.0 mM, 1.2 mM, 1.4 mM, 1.6 mM, 1.8 mM, 2.0 mM, 2.2 mM, 2.4 mM, 2.6 mM, 2.8 mM, 3 mM, 3.2 mM, 3.4 mM, 3.6 mM, 3.8 mM, or 4 mM, may be used.

In preferred aspects UV radiation dosage is between 1 and 12 J/m². For example, around 1, 2, 3, 4, 5, 6, 7, 8, 9, 10, 11 or 12 J/m².

In preferred aspects ionising radiation dosage is between 1 and 50 Gray. For example, about 1, 2, 3, 4, 5, 6, 7, 8, 9, 10, 15, 20, 25, 30, 35, 40, 45, or 50 Gray.

The DNA damage inducing agents may be used alone, or in combination with one or more other DNA damage inducing agents.

### Activation of ATR Kinase

Certain methods described herein involve the activation of ATR kinase. The methods may involve the activation of CHK1 kinase Activation may mean that the kinase is phosphorylated.

The ATR dependent response may be activated through activation of the ATR kinase. For example, though the binding of ETAA1 (Ewing's tumor-associated antigen 1) or a fragment thereof to ATR. In some cases, RS is induced in a cell by contacting the cell with an ATR agonist or a CHK1 agonist.

ETAA1 is deposited as Accession: NP_061875.2 GI: 37059814. The sequence is provided herein as SEQ ID NO:1.

Fragments of ETAA1 useful in the methods described herein include the ATR activating domain (ETAA1-AAD). Useful fragments are referred to herein as “ATR binding fragments”. Such fragments may have at least 80%, at least 85%, at least 90%, at least 95%, at least 98%, at least 99% or 100% identity to a portion of the full length ETAA1 sequence. Such fragments may bind to ATR. Such fragments may activate or induce ATR signalling.

ETAA1 or an ATR binding fragment thereof may be administered to the cells in an amount sufficient to initiate the expression of 2C-like genes in the cell. The ETAA1 or ATR binding fragment thereof may be administered to cells that leads to a level that is at least 1.1 times the amount in a comparator cell, such as the pluripotent stem cell prior to administration, or in another pluripotent stem cell. More preferably, the level may be selected from one of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4 at least 2.5, at least 2.6, at least 2.7, at least 2.8, at least 2.9, at least 3.0, at least 3.5, at least 4.0, at least 5.0, at least 6.0, at least 7.0, at least 8.0, at least 9.0, or at least 10.0 times that in the comparator cell.

An effective amount of nucleic acid encoding ETAA1 or an ATR binding fragment thereof administered to cells may be an amount that leads to a level of nucleic acid encoding ETAA1 or an ATR binding fragment thereof in a cell that is at least 5% more over normal endogenous levels of the nucleic acid (e.g. RNA transcript) in the cell, or alternatively one of at least 10%, 15%, 20%, 25%, 30%, 35%, 38%, 40%, 45%, 50%, 55%, 60%, 65%, 70%, 75%, 80%, 85%, 90%.

Some methods of the present invention involve the introduction to cell(s) of nucleic acid encoding ETAA1 or an ATR binding fragment thereof, such that an effective amount is expressed in the cell(s). Some methods of the present invention involve expressing in the cell an effective amount of ETAA1 or an ATR binding fragment thereof or a nucleic acid encoding ETAA1 or an ATR binding fragment thereof.

In some embodiments an effective amount may be provided by overexpression of ETAA1 or an ATR binding fragment thereof.

Over-expression of ETAA1 or an ATR binding fragment thereof or of a nucleic acid encoding ETAA1 or an ATR binding fragment thereof comprises expression at a level that is greater than would normally be expected for a cell of a given type. This may involve an increase in transcription of a polynucleotide into mRNA and/or the process by which the transcribed mRNA is translated into peptides, polypeptides or proteins that is greater than a base line expression in a cell of an endogenous polynucleotide or gene or one that is exogenous and expressed at base line levels.

As such, over-expression may be determined by comparing the level of expression of a marker between cells that have been transformed with exogenous ETAA1 or an ATR binding fragment thereof or nucleic acid encoding ETAA1 or an ATR binding fragment thereof, or have been induced to overexpress endogenous ETAA1 or an ATR binding fragment thereof, with a cell of the same type that has not been so transformed or induced.

Levels of expression may be quantitated for absolute comparison, or relative comparisons may be made.

In some embodiments over-expression may be considered to be present when the level of expression in a cell is at least 1.1 times that in a comparator cell of the same type. More preferably, the level of expression may be selected from one of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4 at least 2.5, at least 2.6, at least 2.7, at least 2.8, at least 2.9, at least 3.0, at least 3.5, at least 4.0, at least 5.0, at least 6.0, at least 7.0, at least 8.0, at least 9.0, or at least 10.0 times that in the comparator cell.

Overexpression may comprise at least 5% more of a nucleic acid encoding ETAA1 or an ATR binding fragment thereof, e.g. RNA transcript, over endogenous levels of RNA transcript, or alternatively one of at least 10%, 15%, 20%, 25%, 30%, 35%, 38%, 40%, 45%, 50%, 55%, 60%, 65%, 70%, 75%, 80%, 85%, 90%.

In some embodiments the effective amount may be provided by increasing the level of expression of the endogenous nucleic acid encoding ETAA1 or an ATR binding fragment thereof. This may also lead to the levels of over-expression described above.

Expression of an effective amount of ETAA1 or an ATR binding fragment thereof may be achieved from an endogenous nucleic acid encoding ETAA1 or an ATR binding fragment thereof (e.g. genomic nucleic acid encoding ETAA1 or an ATR binding fragment thereof) induced to overexpression or from an exogenous ETAA1 or an ATR binding fragment thereof polynucleotide or an equivalent thereof introduced into the cell and expressed in the cell.

Induction of overexpression of endogenous nucleic acid encoding ETAA1 or an ATR binding fragment thereof may involve inserting a regulatory element, e.g. enhancer element, in the genome of the cell such that it is operably linked to the genomic nucleic acid encoding ETAA1 or an ATR binding fragment thereof leading to upregulation of transcription of the ETAA1 or an ATR binding fragment thereof gene and ETAA1 or an ATR binding fragment thereof overexpression in the cell. Methods of this invention include the introduction of transgenes that are inducible by, for example, chemical agents or physical agents. In this instance, ETAA1 or an ATR binding fragment thereof can be made to be overexpressed in the cell, thereby causing the reprogramming of a non-pluripotent cell to the pluripotent cell.

In some cases, ETAA1 or an ATR binding fragment thereof may be administered to the cells using a lentiviral vector.

### ETAA1 and ATR Binding Fragments Thereof

As described herein, methods of the invention may involve overexpression of ETAA1 or an ATR binding fragment thereof.

ETAA1 is deposited as Accession: NP_061875.2 GI: 37059814. ETAA1 may have the sequence shown in SEQ ID NO: 1.

In some cases, the polypeptide is a fragment of ETAA1. The fragment may be an ATR binding fragment of ETAA1. Methods of determining whether or not a polypeptide is capable of binding to ATR are known in the art. For example, Surface Plasmon Resonance SPR may be used.

In certain aspects the invention concerns expression of peptides/polypeptides comprising an amino acid sequence having a sequence identity of at least 70% with a given sequence. Alternatively, this identity may be any of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity.

Percentage (%) sequence identity is defined as the percentage of amino acid residues in a candidate sequence that are identical with residues in the given listed sequence (referred to by the SEQ ID No.) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

For example, where a given sequence comprises 100 amino acids and the candidate sequence comprises 10 amino acids, the candidate sequence can only have a maximum identity of 10% to the entire length of the given sequence. This is further illustrated in the following example:

(A)

| | |
|---|---|
| Given seq: | XXXXXXXXXXXXXXX (15 amino acids) |
| Comparison seq: | XXXXXYYYYYYY (12 amino acids) |

The given sequence may, for example, be that
encoding ETAA1 (e.g. SEQ ID No. 1).
% sequence identity = the number of identically
matching amino acid residues after alignment
divided by the total number of amino acid residues
in the longer given sequence, i.e. (5 divided by
15) × 100 = 33.3%

Where the comparison sequence is longer than the given sequence, sequence identity may be determined over the entire length of the given sequence. For example:

(B)

| | |
|---|---|
| Given seq: | XXXXXXXXXX (10 amino acids) |
| Comparison seq: | XXXXXYYYYYYZZYZZZZZZ |
| | (20 amino acids) |

Again, the given sequence may, for example, be
that encoding ETAA1 (e.g. SEQ ID No. 1).
% sequence identity = number of identical amino
acids after alignment divided by total number of
amino acid residues in the given sequence, i.e.
(5 divided by 10) × 100 = 50%.

Alignment for purposes of determining percent amino acid sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

Identity of nucleic acid sequences may be determined in a similar manner involving aligning the sequences and introducing gaps if necessary, to achieve the maximum sequence identity, and calculating sequence identity over the entire length of the respective sequences. Where the aligned sequences are of different length, sequence identity may be determined as described above and illustrated in examples (A) and (B).

A fragment may comprise a nucleotide or amino acid sequence encoding a portion of the corresponding full length sequence. In this specification the corresponding full length sequence may be one of SEQ ID NO: 1. Said portion may be of defined length and may have a defined minimum and/or maximum length. Fragments are capable of binding to ATR.

Accordingly, the fragment may comprise at least, i.e. have a minimum length of, 10, 11, 12, 13, 14, 15, 16, 17, 18, 19, 20, 25, 30, 40, 50, 60, 70, 80, 85, 90, 95, 96, 97, 98 or 99% of the corresponding full length sequence. The fragment may have a maximum length, i.e. be no longer than, 10, 11, 12, 13, 14, 15, 16, 17, 18, 19, 20, 25, 30, 40, 50, 60, 70, 80, 85, 90, 95, 96, 97, 98 or 99% of the corresponding full length sequence.

The fragment may comprise at least, i.e. have a minimum length of, 10 nucleotides or amino acids, more preferably at least 15, 20, 25, 30, 40, 50, 100, 150, 200, 300, 400, 500, 600, 700, 800, 900 amino acids.

The fragment may have a maximum length of, i.e. be no longer than, 10 nucleotides or amino acids, more preferably no longer than 15, 20, 25, 30, 40, 50, 100, 150, 200, 300, 400, 500, 600, 700, 800 or 900 amino acids.

The fragment may have a length anywhere between the said minimum and maximum length.

### Uses

2C-like cells produced by the methods disclosed herein may be used as cell models. In particular, 2C-like cells produced by the methods disclosed herein may be used as a model useful for research into early embryonic development, including the study of diseases and disorders associated with early embryonic development.

2C-like cells produced by the methods disclosed herein may also be used to screen for agents or factors (such as solvents, small molecule drugs, peptides, polynucleotides, and the like) or environmental conditions (such as culture conditions or manipulation) that affect the characteristics of the respective cells. Particular screening applications relate to the testing of pharmaceutical compounds in drug research. The reader is referred generally to the standard textbook “In vitro Methods in Pharmaceutical Research”, Academic Press, 1997, and U.S. Pat. No. 5,030,015), as well as the general description of drug screens elsewhere in this document. Assessment of the activity of candidate pharmaceutical compounds generally involves combining the respective cells with the candidate compound, determining any change in the morphology, marker phenotype, or metabolic activity of the cells that is attributable to the compound (compared with untreated cells or cells treated with an inert compound), and then correlating the effect of the compound with the observed change.

Methods disclosed herein may involve separating the 2C-like cells. The 2C-like cells may be separated from pluripotent stem cells in culture that have not been induced to express 2C-like genes. The 2C-like cells may be separated by techniques known in the art, such as FACS. The 2C-like cells may be separated from other cells on the basis of the expression of one or more 2C-like genes. For example, 2C-like cells may be separated from pluripotent cells by separating the cells that express Zscan4 genes, and thereby obtaining a population of 2C-like cells. Thus, in some cases, a population consisting substantially of 2C-like cells, or a population of induced totipotent stem cells is provided.

Such methods may be used to obtain a population of cells that substantially all express 2C-like genes. The population of cells may contain around 99%, 98%, 97%, 96%, 95%, 94%, 93%, 92%, 91%, 90% 85%, 80%, 75%, 70% 2C-like cells.

Methods according to the present invention may be performed in vitro or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms.

In some aspects, 2C-like gene expression is transiently induced. Transient induction of 2C-like gene expression may be useful for maintaining the stability of a culture of pluripotent stem cells. Thus, in some aspects, a composition of pluripotent stem cells with increased genomic stability is provided.

In some methods, 2C-like gene expression is induced in order to increase the developmental potential of the cell population. For example, by inducing the cells to produce trophoblast or other extra-embryonic tissue. Thus, in some methods described herein, 2C-like cells may be used for the production of trophoblast or placental tissue.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1: Transition to Zscan4 state is associated with DNA replication stress in ESC. a, A schematic presentation for inducing replication stress in morula stage embryos through APH treatment. b,c qPCR analysis on blastocyst stage embryo for the key 2C-like specific markers, Zscan4 and Tcstv3. d, A schematic presentation for FACS analysis of Zscan4-Emerald ESC in the Zscan4+ cells and Zscan4− cells. e, FACS analysis on Zscan4-Emerald ESC for DNA damage marker γH2AX and P-CHK1 f, FACS analysis on Zscan4-Emerald ESC upon treatment with wide range of DNA replication stress inducing agents. g, qPCR analysis for MuERV-L gene upon treatment with APH and HU. h,j, immunostaining and qPCR analysis for the canonical pluripotency markers upon treatment with APH. J, qPCR for two key 2C-specific genes; MuERV-L and Zscan4d on E14 lines treated with variety of DNA damage inducing agents.

FIG. 2: ATR mediated replication stress response induces transcriptional signature of 2C-like cells in ESC. a, FACS analysis on Zscan4-Emerald ESC upon treatment with specific ATR and ATM inhibitors. b, Immunoblot for the phosphorylation status of key DDR kinases (CHK1 and CHK2) upon APH treatment in ESC. c, d, qPCR analysis for the key 2C-like specific genes upon treatment with ATR inhibitor. e, FACS analysis on Zscan4-Emerald ESC upon treatment with specific CHK1 inhibitor. f, Zscore-heatmap for the differentially expressed genes upon APH treatment. g, Venn diagram shows the overlap of APH induced DEG with 2C-like specific genes reported in the literature. h, qPCR validation for the main 2C-like specific gene upon APH treatment. i, Heatmap shows the number of APH induced 2C-like gene which have transcriptional reverted their expression upon ATR inhibitor or Caffeine.

FIG. 3: ATR Seckel and CHK1 heterozygous ESC have significantly lower expression level of totipotency genes upon replication stress. a, b, Immunostaining on ATR Sec and heterozygous CHK1 lines for the canonical pluripotency markers. c, d, Immunoblot for ATR and the phosphorylation status of CHK1 under normal and upon APH treatment in ATR Sec and heterozygous CHK1 ESC, respectively. e,f, qPCR for the key 2C-like specific genes in ATR Sec and CHK1 heterozygous ESC. g,h, FACS analysis on Zscan4-Emerald ESC for Emerald-GFP and γH2ax upon infection with two independently produced ETAA1-AAD lentiviruses.

FIG. 4: ATR activated 2C-like cells gain expanded developmental potential. a, Volcano plots generated based on geneset enrichment analysis for the gene categories that are significantly altered upon APH treatment and their position upon ATR or Caffeine. Two placenta-related gene categories are highlighted in red. b, qPCR assay for the giant trophoblast cell specific marker, Prl2c2 in wild-type and ATR Sec ESC derived giant trophoblast-like cells upon APH treatment. c, Phase contrast image and immunostaining of wild-type and ATR Sec ESC derived giant trophoblast-like cells upon APH treatment for PLET-1. d, The scatter plot shows the number of giant trophoblast-like cells generated from wild-type and ATR Sec ESC upon APH treatment. e, A schematic presentation for preparation of ESC before microinjection into morula-stage embryo. f. Contribution of the mCherry positive ESC into the inner cell mass and trophectoderm layer of the blastocysts upon treatment with APH. g. Model for the induction of 2C-like features through the activation of ATR during embryonic development.

FIG. 5: Wide range of DNA replication stress inducing agents trigger activation of 2C-specific genes in ESC. a,b, qPCR analysis on blastocyst stage embryo for the key 2C-like specific markers, Eif1a and GM12794. c, Cell cycle profile analysis of ESC upon treatment with APH. d, qPCR analysis for MuERV-L gene upon treatment with APH in two MEF lines.

FIG. 6: Reactivation of 2C-like specific genes are regulated through ATR. a, FACS analysis on Zscan4-Emerald-GFP ESC upon treatment with ATM and ATR inhibitors after replication stress induction by HU. b, FACS analysis on Zscan4-Emerald-GFP ESC upon treatment with alternative specific ATR inhibitor after replication stress induction c,d, qPCR assay for the key 2C-like specific genes upon HU treatment. e, FACS analysis for gamaH2ax positive cells upon treatment with ATR and ATM inhibitors after induction of replication stress by APH. f, Camera heat map based on geneset enrichment analysis upon ATR and Caffeine treatment after induction of replication stress by APH.

FIG. 7: Expression of 2C-like specific genes are regulated ATR. a,b, PCR genotyping assay on ARE Sec and CHK Het ESC lines c,d, expression of the key totipotency specific genes in ATR Sec and CHK1 Het ESC. e, Cell cycle profile of ETAA1-ADD overexpressing ESC.

FIG. 8: ATR induce trophectoderm directed differentiation program.

FIG. 9: Induction of RS increases the number of 2C-like cells in ESCs culture and activates the expression of 2C-like specific genes in mouse embryos. a. Clustering of 1400 Drop-seq single-cell expression profiles into four cell populations. The plot shows a two-dimensional representation (t-SNE) of global gene expression relationship; clusters are colored by cell class. b, Two-dimensional representation (t-SNE) of global gene expression relationship among 1400 cells; clusters are colored by biological sample. c, PCA on 1399 analyzed cells. Cells are colored by cluster. d, PCA on 1399 analyzed cells. Cells are colored by experimental condition. e, Heatmap showing the list of genes that are differentially expressed between cluster 4 cells (orange in FIG. 1a) and the rest of the population (cluster 1, 2, and 3). f-k, t-SNE plots showing the expression level of representative 2C-like genes across all CNTL and APH treated cells. j, Venn diagram showing the overlap of DEGs of cluster 4 with those expressed in 2C-like cells. i, FACS analysis on pZscan4-Emerald ESCs upon treatment with a wide range of RS inducing agents. m, Immunoblot showing the effect on p-ATM, t-ATM, p-Chk2, p-Chk1, t-Chk1, GAPDH. n. FACS analysis on pZscan4-Emerald ESCs upon treatment with a wide range of RS inducing agents, UV, IR, HU, and Aph. o, qPCR analysis for Zscan4d expression upon treatment with a wide range of RS inducing agents. p, FACS analysis for Emerald-GFP and CASPASE 3 upon increasing concentration of APH. q, qPCR analysis for MERVL element expression upon treatment with increasing concentrations of APH. r, qPCR analysis for MERVL element expression upon treatment with a wide range of RS inducing agents. s, qPCR analysis for Dux upon treatment with increasing concentrations of APH t, qPCR analysis for MERVL upon treatment with APH in two different MEF lines. u, v, Immunostaining and qPCR analysis of ESCs for canonical pluripotency markers upon treatment with APH. w, Cell cycle analysis of E14 and R1 ESCs upon treatment with low (0.3 μM) and high (6 μM) concentration of APH. x, Wide field microscope images of mouse embryos at blastocyst stage after APH treatment. y, qPCR analysis on blastocyst stage embryos for the key 2C-like markers. All bar-plots show mean with SD (*P≤50.05, **P≤50.01, ***P≤50.001, one-way ANOVA). z, A replicate of qPCR analysis on blastocyst-stage embryos for key 2C-like specific markers Dux, MERVL and Gm4981. All bar plots show mean with SD (*P≤50.05, **P≤50.01, ***P≤50.001, one-way ANOVA).

FIG. 10: ATR and CHK1-mediated RSR triggers activation of key 2C-like specific genes in ESCs. a, FACS analysis on pZscan4-Emerald ESCs showing the number of Em⁺ cells upon treatment with APH and specific ATR and ATM inhibitors. b, FACS analysis of pZscan4-Emerald ESCs upon treatment with ATM and ATR inhibitors after HU induced RS. c, FACS analysis of pZscan4-Emerald ESCs for DNA damage markers γH2AX and p-CHK1 (Em⁺ and Em⁻ correspond to Emeraled-GFP positive and negative population, respectively). d, Immunoblot for the phosphorylation status of key DDR kinases (CHK1 and CHK2) and the ZSCAN4 protein upon treatment with APH and ATM/ATR inhibitor in ESCs. e, qPCR analysis in two ESCs lines for the 2C-like specific element, MERVL upon treatment with APH and ATRi. f, qPCR analysis for Zscan4d gene upon treatment with a specific ATRi in two distinct ESC lines (E14 and R1). g, t-SNE plot showing the global gene expression relationship among 2100 cells; clusters are colored based on biological sample. h, tSNE plots showing the expression level of Zscan4d across all sequenced cells, with pullout showing the contribution of cells coming from CNTL, APH and APH+ATR conditions to Zscan4d cell population within the newly emerging cluster. i, j, tSNE plots showing the expression level of representative 2C-like genes across all sequenced cells. All bar plots show mean with SD (*P≤50.05, **P≤50.01, ***P≤50.001, one-way ANOVA). k, Immunostaining of ATR^{Sec/Sec} and ATR^{+/+} ESCs for the canonical pluripotency markers POU5F1 and NANOG. l, Immunoblot for ZSCAN4, ATR and the phosphorylation status of CHK1 upon APH treatment of ATR^{Sec/Sec} and ATR^{+/+} ESCs. m, Immunostaining of CHK1^{-/+} and CHK1^{+/+} ESCs for canonical pluripotency markers. n, qPCR analysis for 2C-like genes in ATR^{Sec/Sec} and ATR^{+/+} ESCs upon treatment with APH. o,p, qPCR for 2C-like specific genes in ATR^{Sec/Sec} and ATR^{+/+} ESCs. q, qPCR analysis for 2C-like genes in Chk1^{+/-} and Chk1^{+/+} ESCs upon treatment with APH. r, FACS analysis on pZscan4-Emerald ESCs showing the number of Em⁺ cells upon treatment with APH and specific CHK1 inhibitor. s, qPCR analysis for Trp53 expression upon siRNA mediated KD. CNTL sample was transfected with sc siRNA. t, Immunoblot showing ZSCAN4 expression and phosphorylation status of P53 and CHK1 upon APH treatment in CNTL and Trp53 KD ESCs.AII bar plots show mean with SD (*P≤50.05, **P≤50.01, ***P≤50.001, one-way ANOVA).

FIG. 11: ATR induces transcriptional signature of 2C-like cells in ESCs through DUX activation. a, Heatmap showing the robust z-scores for all the DEGs in three ESCs lines upon treatment with APH or APH-ATRi. b, MA plot showing global activation of 2C-like specific genes upon APH treatment. c, MA plot showing activation of 2C-like specific retrotransposons upon APH treatment. d, Validation of RNA-Seq results by qPCR analysis. e, Heatmap showing the robust z-scores for 2C-like specific genes in the indicated samples. 2C-like specific genes were identified based on performing a differential expression analysis on Z⁺ v.s. Z⁻ ESCs from Eckersley et. al.,¹³ f, Plot shows the number of genes expressed in ESCs upon treatment with APH overlapping with previously published datasets. The −log 10(p-value) is the result of the Fisher test used to test the significance of the overlap. g, Bar plot displaying the percentage of ATR dependent differentially expressed genes among the ones shared between APH treated ESCs and each dataset. h-j, qPCR analysis for Dux, MERVL and Zscan4 markers upon treatment with APH and ATRi in Dux KO and WT ESCs. All bar plots show mean with SD (*P≤50.05, **P≤50.01, ***P≤50.001, one-way ANOVA).

FIG. 12: ETAA1-mediated direct activation of ATR induces 2C-like cells in a RS free context. a, full length human ETAA1 sequence of SEQ ID NO: 1 b, ETAA1-ADD sequence of SEQ ID NO: 2. c, nucleic acid encoding ETAA1 d, nucleic acid encoding ETAA1-ADD e, FACS analysis for γH2AX and Emerald-GFP upon overexpression of ETAA1-AAD by Dox in two different lentivirus clones with respect to empty vector-infected ESCs. f, FACS analysis for γH2AX and Emerald-GFP in Dox-iETAA1 ESCs in the presence or absence of Dox and upon treatment with ATRi or ATMi. g, h, qPCR results for MERVL element and Dux gene in Dox-iETAA1 ESCs upon Dox induction in the presence or absence of ATRi or ATMi. i, Immunoblot showing the expression of ZSCAN4 and the phosphorylation status of CHK1, CHK2 and H2AX in Dox-iETAA1 ESCs upon treatment with Dox, ATRi or ATMi. j, k, l, m, qPCR results for Dux and Zscan4 expression in Dox-iETAA1 ESCs after 24, 48 and 72 hrs of Dox administration. n, o, qPCR results for 2C-like genes, Zscan4 and Dux in Dox-iETAA1 ESCs upon Dox and CHK1i treatment. p, Immunoblot for ZSCAN4 in Dox-iETAA1 ESCs upon Dox induction and Dux kKD. CNTL sample was transfected with scrambled siRNA (sc siRNA). q, qPCR analysis for MERVL, Zscan4 and Dux in Dox-iETAA1 ESCs upon induction with Dox and Dux KD. CNTL sample was transfected with sc siRNA. All bar plots show mean with SD (*P≤50.05, **P≤50.01, ***P≤50.001, one-way ANOVA).

FIG. 13: ATR-activated 2C-like cells gain expanded developmental potential in vitro and in vivo. a, Phase contrast images and immunostaining of ATR^{+/+} and ATR^{Sec/Sec} TGCs upon APH treatment. b, qPCR assay for the TGCs specific marker Pr/2c2 in ATR^{+/+} and ATR^{Sec/Sec} TGCs upon APH treatment. c, Plot showing the number of TGCs generated from ATR^{+/+} and ATR^{Sec/Sec} ESCs upon APH treatment. d, Plot showing the number of TGCs generated from WT and Dux KO ESCs upon APH treatment. e, Immunostaining of WT and DUX KO TGCs upon APH treatment. f, Images of blastocysts displaying the contribution of mCherry labeled Dox-iETAA1 ESCs to the ICM and TE layers in the presence and absence of Dox. g, Bar plot showing the percentage of chimeric embryos in which injected mCherry-labeled Dox-iETAA1 ESCs could contribute to either ICM or ICM+TE with or without Dox. The ratios on top of each bar show the actual number of embryos analyzed. h, Contribution of injected mCherry labelled ESCs treated with or without APH to the ICM or TE layers of the blastocyst. i, Images showing the contribution of injected mCherry-labeled Dox-iETAA1 ESCs to the epiblast (EPI) or extra-embryonic layers (EEL) of mouse embryos at E7.5 with or without Dox treatment. j, Schematic model defining a novel ATR-dependent transcriptional response to maintain the genomic integrity of developing embryos in response to RS: I) ATR and CHK1-mediated RSR triggers Dux expression that in turn increases bipotent 2C-like cells by global transcriptional activation of 2C-like specific genes including Zscan4. II) ATR-induced bipotent ESCs extend their contribution to placental compartment. All bar plots show mean with SD (*P≤50.05, **P≤50.01, ***P≤50.001, one-way ANOVA).

FIG. 14: Activation of genes involved in the invasiveness and immunoediting by RS. a, FACS analysis shows activation of PDL-1 upon replication stress in mESC. b, FACS analysis shows activation of PDL-1, GARP and BTLA upon replication stress in hESC.

### EXAMPLES

### Example 1: Materials & Methods

### Cell Culture

ESCs were grown in feeder free culture condition either with or without 0.1% gelatin coat and incubated in 37° C. and 3% 02 tension. ESC medium (high glucose DMEM (DMEM Media—GlutaMAX™-I, Gibco)), 15% ESC qualified FBS (HyClone™ Fetal Bovine Serum), 2 mM L-glutamine, 1/500 home-made leukemia inhibitory factor, 0.1 mM non-essential amino acids, 0.1 mM 2-bmercaptoethanol, 50 units/ml penicillin, 50 mg/ml streptomycin, 1 mM Sodium Pyruvate) supplemented with two inhibitors (2i from Axon medchem) that are PD 0325901; highly specific non-ATP-competitive inhibitor of MEK (aka MKK) 1/2 at final concentration of 1 uM and CHIR 99021; specific glycogen synthase kinase GSK-3 inhibitor at final concentration of 3 uM. MEF cells were cultured in MEF medium (high glucose DMEM (Lonza), 10% north America FBS, 2 mM L-glutamine, 0.1 mM non-essential amino acids, 50 units/ml penicillin, 1 mM Sodium Pyruvate and 50 mg/ml streptomycin).

### Treatments

All treatments including APH, HU, UV, ATRi, ATMi and Chk1i were added in to fresh ESC medium and the cells were kept in 37° C. and 3% 02 tension for overnight (16-17 hrs) unless mentioned in each particular result section. APH was used at various concentrations ranging from 0.3 uM to 6 uM that is mentioned in each experiment in order to induce mild to acute replication stress response. Similarly, HU was used at 0.1 mM-3 mM. ATMi (KU-55933) and ATRi (VE 822) were used at the concentration of 10 uM and 1 uM respectively. Moreover, Chk1i (UCN-01) at the concentration of 100 nM was used. UV radiation was performed at the dosage ranging from 2 to 10 J/m2. For induction of ETAA1-AAD expression, Dox was used at the concentration of 1 μg/mL for 48 hrs.

### Flow Cytometry (FACS)

ESCs were fixed and permeabilized using Cytoperm/Cytofix kit (BD Biosciences), and subsequently stained for one hour at room temperature with Alexa Fluor® 647 anti-H2AX-Phosphorylated (Ser139) antibody (Biolegend) or P-CHK1-PE conjugated antibody (cell signaling 12268). Cells were washed and acquired on a FACS Calibur instrument or Attune NxT (Thermo Fisher Scientific). For Emerald-GFP acquisition, cells were trypsinized, collected and subsequently acquired without fixation. Cell cycle profiles were acquired after incubation with DAPI (4′,6-diamidino-2-phenylindole) overnight. For Caspase3 FACS analysis after fixation and permeabilization, Cleaved Caspase3 antibody were used for one hr at RT followed by washes and one hr incubation in donkey anti rabbit secondary antibody. For Zscan4-Emerald-GFP acquisition cells were trypsinized, collected and subsequently acquired without fixation step.

RNA Extraction, cDNA Synthesis and qPCR

RNA was extracted using RNeasy mini kit (QIAGEN), quantified by NanoDrop spectrophotometer and cDNA was prepared from 2 μg total DNA-depleted RNA using SuperScript™ III reverse transcriptase kit (Invitrogen cat #18080-093) following manufacturer's instructions. qPCR assay performed based on standard protocol using 10× SsoFast™ EvaGreen® Supermix or later 1× LightCycler 480 SYBR Green I Master (Roche, 04707516001), 0.5 μM primer mix and 5 ng of cDNA. GAPDH was used as internal control to normalise the qPCR data following ΔΔCₜ method, 10 μM primer mix and a total amount of cDNA corresponding to 5 ng of starting RNA for each reaction.

RNA Isolation from Embryos and cDNA Preparation

Cryopreserved C57BL/6N morulae purchased from Janvier Labs (QUICKBLASTO®), were thawed according to the manufacturer's protocol. After recovery, a group of healthy morulae were treated with APH at final concentration of 1.5 μM in KSOM culture medium for 4 hrs while the rest were kept as control. The day after, the same number of morphologically healthy and synchronized blastocysts (22 and 7 blastocysts in the 1^{st} and 2^{nd} round, respectively) were selected from both groups. The APH concentration and the treatment interval had no effect on the viability of the embryos. RNA isolation was performed based on the previously published works^{14,15}. Briefly, following the removal of zona pellucida in acidic tyrode's solution (Sigma-Aldrich, T1788), the pool of embryos was collected in 10 μl of lysis buffer containing 5× First Strand Buffer (from SuperScript III reverse transcriptase kit, ThermoFisher) supplemented with 0.1% Tween-20. Subsequently, embryos were mechanically ruptured by three cycles of freeze/thaw. As the quantity of isolated RNA is not detectable by NanoDrop spectrophotometer, the supernatant was directly used for cDNA preparation following centrifugation at 10621 g for 2 minutes. cDNA preparation and qPCR were performed following the aforementioned protocols.

### Immunocytochemistry

Briefly cells fixed in 4% PFA and subsequently blocked for one hour in FBS (10%) and Triton (0.1%). Then, cells were incubated with primary antibody at 4° C. overnight, followed by washes and incubation with secondary antibody for an hour at room temperature. Next samples were mounted and images were acquired with wide field florescent microscope. Antibodies used in this study were Anti Oct-3/4 (Santa Cruz sc-5279), Anti Nanog (Abcam ab80892) and Plet-1 antibody (Nordic MUbio MUB1512P) and), Anti-GFP (Abcam, ab5450). Acquired figures were analyzed with ImageJ software.

### Immunoblotting

The cells were trypsinized and washed with PBS and then lysed in RIPA buffer plus protease/phosphatase inhibitor cocktail (cell signaling, #5872), incubating for 30 minutes on a rotating wheel at +4° C. Lysates were sonicated with a Bioruptor Sonication System (UCD200) at high power for 3 cycles of 30 seconds with one minute breaks. Lysates were centrifuged at 13000 rpm for 20-30 minutes and clear supernatants were transferred to new tubes. The proteins were quantified using Bio-Rad protein assay and following manufacturer's instructions. For the detection of each protein, 35 μg of total protein extracts were loaded. Standard western blot was performed using following antibodies: ATR antibody (Cell Signaling 2790), Phoshpho-Chk1 antibody-S345 (Cell signaling 2348), Phoshpho-Chk1 antibody S317 (cell signaling 12302), Zscan4 antibody (Abcam ab4340), total Chk1 antibody (Santa Cruz 8408) and Chk2 antibody (Millipore 05-649), Anti-P53 (Cell Signalling, 2524S) and Anti-p-P53 (S15) (Cell Signalling, 12571S), Anti-Goat ALexa488 (Thermo Fisher Scientific, A-11055), Anti-Rabbit Cy3 (Jackson ImmunoResearch, 711-165-152), Anti-Mouse Alexa647 (Thermo Fisher Scientific, A-31571).

### Target Preparation for Microarray

The quality of total RNA was first assessed using an Agilent Bioanalyzer 2100 (Agilent Technologies, Palo Alto, Calif.). Biotin-labeled cDNA targets were synthesized starting from 150 ng of total RNA. Double stranded cDNA synthesis and related cRNA was performed with GeneChip® WT Plus Kit (Affymetrix, Santa Clara, Calif.). With the same kit was synthesized the sense strand cDNA before to be fragmented and labeled. All steps of the labeling protocol were performed as suggested by Affymetrix. Each eukaryotic GeneChip® probe array contains probe sets for several B. subtilis genes that are absent in the samples analyzed (lys, phe, thr, and dap). This Poly-A RNA Control Kit contains in vitro synthesized, polyadenylated transcripts for these B. subtilis genes that are pre-mixed at staggered concentrations to allow GeneChip® probe array users to assess the overall success of the assay. Poly-A RNA Controls final concentration in each target are lys 1:100,000, phe 1: 50,000, thr 1:25,000 and dap 1:6,667.

### DNA Strip Hybridization

Hybridization was performed using the GeneAtlas™ Hybridization, Wash and Stain Kit. It contains a mix for target dilution, DMSO at a final concentration of 10% and pre-mixed biotin-labeled control oligo B2 and bioB, bioC, bioD and Cre controls (Affymetrix cat #900299) at a final concentration of 50 pM, 1.5 pM, 5 pM, 25 pM and 100 pM respectively. Targets were diluted in hybridization buffer at a concentration of 0.05 μg/ul, denatured at 99° C. for 5 minutes, incubated at 45° C. for 5 minutes and centrifuged at 5,000 rpm for 1 minute. The Array Strip is then moved to a hybridization tray, which contains the hybridization cocktails. A single Gene 2.1 PEG Array Strip is hybridized with four different biotin-labeled targets. Hybridizations were performed for 20 hours at 48° C. in the GeneAtlas™ Hybridization Station. The Array Strips were washed and stained in the GeneAtlas™ Personal Fluidics Station. At the end of this procedure the array strips were placed into the imaging tray, which contains the Array Holding buffer.

### Image Acquisition, Processing and Bioinformatics Analysis

The array strips were imaged using the GeneAtlas™ Imaging Station. Affymetrix GeneAtlas™ Command Console software was used to acquire the Array Strip images and generate.

### Differential Expression Analysis

We imported the microarray CEL files into R-3.2.2 (R Core Team. 2015. R: A Language and Environment for Statistical Computing. https://www.r-project.org/ (Accessed Sep. 12, 2016) and normalized intensity values with RMA 26 normalization. We performed differential expression analysis using the limma 27 package, weighting arrays with the arrayWeights function 28 and calculating p-values with an empirical Bayes method 29.

Expression Z-scores were calculated using the probe set level RMA normalized microarray intensity values using the standard formula:

Z-score=(x−μ)/σ

Where x is the RMA normalized microarray intensity, μ is the probeset level average intensity value across all samples, and σ is the probeset level standard deviation of the intensity values.

### Gene Set Enrichment

The CAMERA gene set enrichment p-values were calculated using the limma package and the camera function 30. Gene sets were defined based on the MsigDB gene sets 31, using a mouse orthologous collection (Hu Y. 2016. Mouse and human orthologues of the MSigDB in R format. http://bioinf.wehi.edu.au/software/MSigDB/ (Accessed Sep. 12, 2016).

In the GSVA based enrichment analysis, we used the MsigDB gene sets, and transformed the individual gene expression values into gene set expression values with the GSVA package 32. We calculated the gene set differential expression as in the gene level differential expression analysis, only skipping the RMA normalization step.

### Genotyping of ATR Seckel ESC

In order to obtain homozygous ATR Seckel ESC, all nine ESC lines derived from breeding of ATR Seckel heterozygous mice were genotyped using previously described primer sets 14. 300 bp and 545 bp bands correspond to WT and ATR Seckel −/− genotypes.

### In-Vitro Differentiation Toward Giant Trophoblast-Like Cells

ATR Seckel and WT ESCs were seeded on gelatin coated 6-well plate in ESC medium and 24 hours post treatment, medium was changed to trophectoderm stem cell (TSC) differentiation medium, which contains: 30% RPMI 1640 (with 20% FBS, 1 mM pyruvate, 2 mM L-glutamine, 100 mM β-mercaptoethanol), 70% of conditioned medium from mitomycin-C-inactivated fibroblasts, 25 ng/ml of FGF4 (R&D Systems, 235-F4-025) and 1 ug/ml of heparin (Sigma, H3149). We changed the medium with fresh one every other day to maintain TSCs. To induce giant cell differentiation established TSC were split at day 2 on gelatin coated plate. After 24 hrs medium was changed to RPMI 1640 (with 20% FBS, 1mMpyruvate, 2mML-glutamine, 100 mM β-mercaptoethanol) in the absence of heparin and FGF4, therefore, we changed the medium every other day for 3 days.

### Ex Vivo Differentiation

Cryopreserved C57BL/6NRj morulas purchased from Janvier lab (QUICKBLASTO), were thawed according to the manufacturer's protocol. After recovery, half of morphologically healthy and synchronized morulas were treated with 1.50 M aphidicolin for four hours and the other half were kept as an untreated control (about 20 embryos in each condition). The day after, the same number of healthy blastocysts (E3.5) were collected from each group for RNA extraction, cDNA preparation and qPCR assay.

### Mouse Chimaera Assay

Briefly, morula stage embryos were bought from Charles River laboratories. They were thawed few hours prior to injection and about five to eight ESC were injected into the perivitelline space of high quality morula-stage embryos using laser-assisted technique and glass micro-capillary tubes. Finally, embryos were cultured for 24 hours at 37 C, 5% CO2 to allow the blastocysts development prior to the microscopic analysis.

For ETAA-1 inducible cells, 8-cell morulae were obtained from superovulated C57BL/6 prepubescent females from Janvier Labs. Ten ESCs were injected into each morula after piercing their zona pellucida with beveled microinjection needle. Embryos were kept in M2 medium during the injection and afterwards in KSOM medium at 37° C. under 5% CO₂. Morulae were either cultured for 48 hrs to study ESC contribution at late blastocyst stage (E4.5), or transferred to pseudopregnant females and later dissected at E7.5. Chimerism and ESC contribution to the various embryonic layers were assessed by emission of mCherry fluorescence signal.

For the APH-treated ESCs, frozen morula-stage embryos were purchased from Charles River and Janvier laboratories. Embryos were thawed 2 hrs prior to injection and 4 to 8 ESCs were transferred into the perivitelline space by laser-assisted microinjection method. Afterwards, embryos were cultured for 24 hrs at 37° C., 5% CO₂ until blastocyst stage (E3.5).

### Lentivirus Production

pLVX-EF1α-IRES-mCherry vector was co-transfected with plasmids that are expressing POL, REV, TAT, and the vesicular stomatitis virus envelope glycoprotein (VSV-G) into 80% confluent 293T cells using calcium phosphate precipitation in the presence of 25 mM chloroquine. The supernatant of transfected cells were collected every 24 hrs for two days and concentrated using PEG-it™ virus precipitation solution and viral particles were re-suspended in DMEM and frozen in small aliquots in −80° C.

### Animals

All mice used to generate ATR Seckel ESCs and MEF lines were bred and maintained under specific pathogen-free conditions. C57Bl/6J and 129P2/OlaHsd mice were purchased from Charles River Laboratories Harlan Italy (currently known as Envigo), respectively. Animals were kept in ventilated cages in standard 12 hrs light-dark cycle. The procedure was approved by the FIRC Institute of Molecular Oncology Institutional Animal Care and Use Committee and performed in compliance with Italian law (D.Igs. 26/2014 and previously D.Igs. 116/92), which enforces Dir. 2010/63/EU (Directive 2010/63/EU of the European Parliament and of the Council of 22 Sep. 2010, on the protection of animals used for scientific purposes).

ESCs Derivation from ATR Seckel and Chk1 Mice

To establish ATR^{Sec/Sec} ESCs, ATR^{+/Sec} heterozygous mice were crossed. Morulae were recovered and cultured overnight in KSOM medium (Millipore, MR-020P-5D) under mineral oil. Blastocysts were placed on MEFs feeder layer in ESC medium with 2i and LIF at 37° C. under 5% CO₂. Upon ICM expansion, cells were passaged on feeder layer a to obtain the first stock. The cells were then characterized by genotyping. To establish Chk1^{+/-} ESCs lines, embryos at morula stage were cultured overnight in KSOM supplemented with 2i. On the following day, blastocysts were plated individually in 96-well plate and cultured in N2B27 medium supplemented with 2i and LIF. After 6-7 days, the ICM outgrowth was disaggregated using Accutase. Clumps of cells were expanded every second day, or when the size of colonies reached to proper expansion level.

### MEFs Generation

To produce MEFs, C57BL/6 and 129P2/OlaHsd mice were used. MEFs were prepared by mechanical disaggregation, trypsinization and seeding of embryos (E12-E13) in MEF medium after removal of the head, tail, limbs, and internal organs. Each trypsinized embryo was plated into a 10 cm dish. Once cells reached to 90% confluency (after 48 hrs), were frozen and stocked for the subsequent experiments.

Drop-Seq Single Cell mRNA Sequencing

ESCs from CNTL, APH (6 μM) and APH-ATRi experimental conditions were resuspended in PBS-BSA and processed with a microfluidic device according to the DropSeq Laboratory Protocol V3.1 from McCarroll's Lab website [http://mccarrolllab.com/dropseq/l]. For each condition, 3 distinct aliquots of collected emulsion, each containing 4000 beads underwent reverse transcription (RT) and fragment library preparation using Illumina Nextera XT Library Prep Kit. A distinct barcode was used for each library to allow subsequent demultiplexing of sequencing reads. mRNA sequencing was performed using an Illumina HiSeq2000 instrument, library fragments were sequenced at 50 base pairs (bp) in PE mode. Sequencing reads were aligned to UCSC Mouse Reference Genome version mm10 using STAR (version 2.5.3a), and processed according to DropSeq Alignment Cookbook V1.2 to generate a digital expression matrix of STAMPs (cells) for each experimental condition, which was then furtherly analyzed in the RStudio environment (R version 3.3.3) using Seurat package V2.0 from Satija Lab [http://satijalab.org/seurat/]. Seven-hundred most expressed cells were selected in each condition; genes expressed in less than 3 cells and cells expressing less than 200 genes were pruned from the dataset. Data from CNTL and APH conditions (1399 cells) or CNTL, APH and APH-ATRi conditions (2096 cells) were merged to generate two datasets, which were log normalised and scaled. Expression mean and variance to mean ratio (Log VMR>0.5) were used to estimate data dispersion and select variable genes across the dataset. Respectively 2614 and 2768 genes (in the 2-conditions and 3-conditions datasets) were used in principal component analysis (PCA) to identify the appropriate number of components to include in data modeling. Top 15 components were selected to explain dataset complexity. A shared nearest neighbor (SNN) graph and smart local moving algorithm was used to perform cells clustering; t-SNE (t-stochastic neighbour embedding) analysis was used to reduce (PCA) dimensionality and generate plots of cells distribution.

### RNA Sequencing Library Preparation and Sequencing

RNA samples were quantified using Qubit 2.0 Fluorometer (Life Technologies, Carlsbad, Calif., USA) and RNA integrity was checked with RNA Screen Tape on Agilent 2200 TapeStation (Agilent Technologies, Palo Alto, Calif., USA). All RNA samples had a RIN score of 10. RNA sequencing library preparation was prepared using Ribo-Zero rRNA Removal Kit and TruSeq Stranded Total RNA library Prep kit following manufacturer's protocol (Illumina, Cat # RS-122-2101). Briefly, rRNA was depleted with Ribo-Zero rRNA Removal Kit. rRNA depleted RNAs were fragmented for 8 minutes at 94° C. First strand and second strand cDNA were subsequently synthesized. The second strand of cDNA was marked by incorporating dUTP during the synthesis. cDNA fragments were adenylated at 3′ends, and indexed adapter was ligated to cDNA fragments. Limited cycle PCR was used for library enrichment. The incorporated dUTP in second strand cDNA quenched the amplification of second strand, which helped to preserve the strand specificity. Sequencing libraries were validated using DNA Analysis Screen Tape on the Agilent 2200 TapeStation (Agilent Technologies, Palo Alto, Calif., USA), and quantified by using Qubit 2.0 Fluorometer (Invitrogen, Carlsbad, Calif.) as well as by quantitative PCR (Applied Biosystems, Carlsbad, Calif., USA).

The sequencing libraries were multiplexed and clustered on 1 lane of flowcell. After clustering, the flowcell was loaded on an Illumina HiSeq 4000 instrument according to manufacturer's instructions. The samples were sequenced using a 2×150 Pair-End (PE) High Output configuration. Image analysis and base calling were conducted by the HiSeq Control Software (HCS) on the HiSeq instrument. Raw sequence data (.bcl files) generated from Illumina HiSeq was converted into fastq files and de-multiplexed using Illumina bcl2fastq program version 2.17. One mis-match was allowed for index sequence identification. On average ˜29 million reads were generated for a sample and average insert size ranged between 204-231 for the samples.

### RNA Sequencing Data Processing

Transcript and gene level quantification was done with Kallisto (version 0.43.0)¹⁶. The RefSeq NM and NR sequence collection as of 2016 Nov. 21, was used to build the transcriptome index. Kallisto was run with the −-bias option to perform sequence based bias correction on each sample. Additionally, all reads were mapped with the STAR spliced aligner (version 2.5.2b)¹⁷ to the GRCm38 (mm10) genome after trimming the reads to 100 bp with Trimmomatic (version 0.36)¹⁸. Repeat expression was quantified by counting fragments overlapping with the repeat annotation from the Choi et al. 2017 paper, with using FeatureCounts from the subread package (version 1.5.2)¹⁹. FeatureCounts was run with the −p −B −C −-primary options.

### RNA Sequencing Gene Level Differential Expression

Read counts for each sample estimated by Kallisto were imported into the R statistical environment (version 3.2.2) using tximport (version 1.1.2)²⁰. The limma (version 3.24.15) package²¹ was used to test for differential expression between CNTL and APH or CNTL and APH+ATRi treatments after TMM normalization and voom transformation²². A linear model was fitted with the limma ImFit function and the moderated t-statistics was calculated with the eBayes function. Genes were defined as differentially expressed if they had FDR adjusted p-values <0.05 and |log₂ fold change|>1.

The robust z-score for each gene in each sample was calculated using the median and mean absolute deviation (MAD) for each gene across samples, and standardizing expression with the following formula:

$$Robust Z - score = \frac{x - median}{MAD}$$

where x is the expression in TPM for a single gene in a given sample.

### RNA Sequencing Repeat Analysis

Read counts for each sample based on the STAR alignment were imported into the R statistical environment (version 3.2.2) and summarized on the repeat subfamily level. edgeR²³ was used to test for differential expression between CNTL and APH treatments, after TMM normalization. All repeat subfamilies were dropped where the count per million value did not reach 1 in at least 2 samples. A linear model was fitted with the edgeR glmFit function and a likelihood ratio p-value was calculated with the glmLRT function. Repeat subfamilies were defined as differentially expressed if they had FDR adjusted p-values <0.05 and |log 2 fold change|>1.

### RNA Sequencing Gene Set Enrichment Analysis

Gene set enrichment was calculated using a mouse version of MSigDB available from the website of the bioinformatics group at the Walter and Eliza Hall Institute (Accessed Sep. 12, 2016). The CAMERA method²⁴ available in the limma package was used to check for significant enrichment of specific gene sets between the CNTL and APH treatments, using the same TMM normalized read counts as in the gene differential expression analysis. Additionally, the GSVA²⁵ package was used to transform gene level read counts per sample to a gene set level enrichment score per sample and for each MSigDB gene set. After this, the same limma analysis was carried out on the gene set enrichment values as for the gene level data. Gene sets were defined as differentially expressed if they had FDR adjusted p-values <0.05 and |log₂ fold change|>0.5.

### RNA Sequencing Literature Comparison

For all of the literature comparisons, the same transcript and gene level quantification methods and transcriptome annotations were used as for our data using the same limma package and methods to define differentially expressed genes between conditions. The Fisher-test was used to check for the significance of the overlaps in differentially expressed genes between our data and the literature datasets.

### Transfection

ESCs were plated in 2i plus LIF medium as described above without antibiotics. Lipofectamine 2000 (Sigma-Aldrich) was diluted 1:50 in Opti-MEM (ThermoFisher) and incubated for 5 minutes at room temperature. Dux MISSION esiRNA pool (Sigma-Aldrich, EMU204411) or Trp53 siRNA (Sigma-Aldrich, SASI_Mm02_00310137) were diluted in Opti-MEM to the opportune concentration and then added to the lipofectamine emulsion at 1:1 ratio. This mixture was incubated for 20 minutes at room temperature and then was added to the cells suspension in culture medium. The final concentration of Dux and Trp53 were 75 nM and 40 nM, respectively. For each experiment a sample was transfected with MISSION siRNA universal negative control (scrambled siRNA) (Sigma-Aldrich, SIC005) at the same concentration used for the esiRNA or siRNA of interest. Gene expression was assessed 48 hrs post transfection.

### ETAA1-AAD Cloning, Lentivirus Production and Induction

Lenti-XTM Tet-On® 3G Inducible Expression System was used to express ETAA1-AAD protein in ESCs. The cDNA of ETAA1-AAD (kind gift from Niels lab) was cloned in pLVX-TRE3G vector. Viral particles of pLVX-TRE3G-ETAA1-AAD and pLVX-EF1a-Tet3G vectors were separately produced (as mentioned above). ESCs were co-infected with both lentiviruses in the presence of 8 μg/mL polybrene, and subsequently infected cells were undergone puromycin and neomycin selection for two weeks. To induce ETAA1-AAD expression, selected ESCs were treated with 1 μg/mL Dox for 48 hrs.

### Example 2: RS Increases 2C-Like Cells in ESCs Culture and Activates 2C-Like Gene Expression in Mouse Embryos

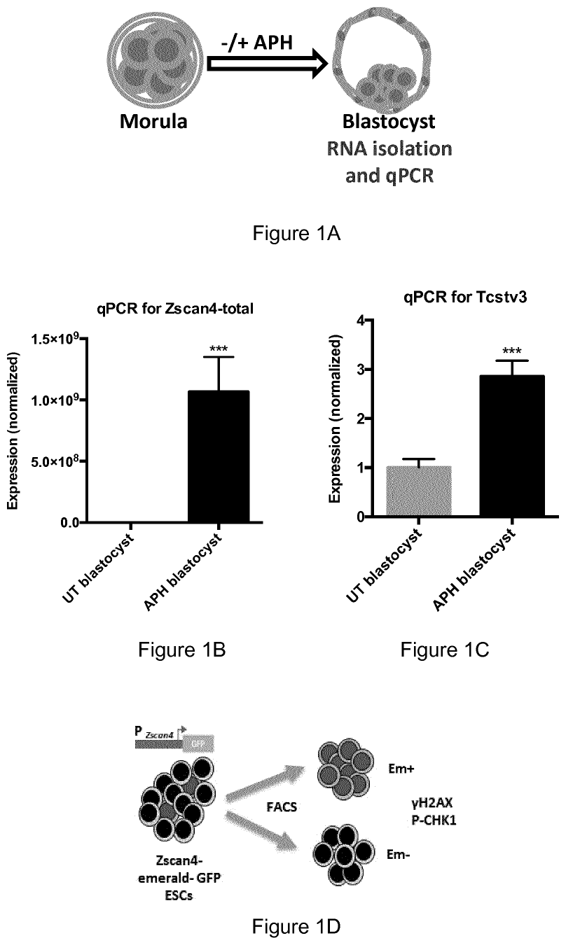

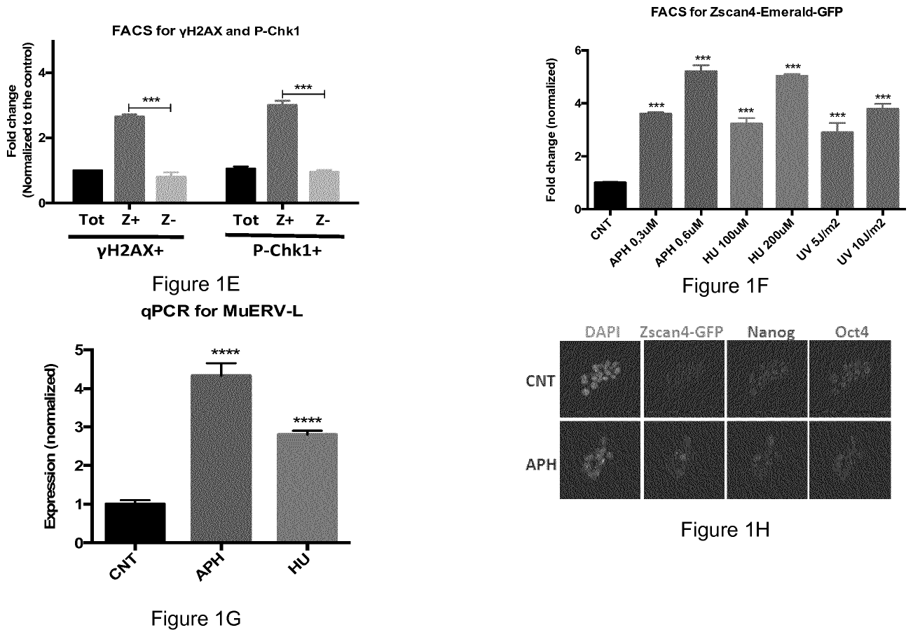

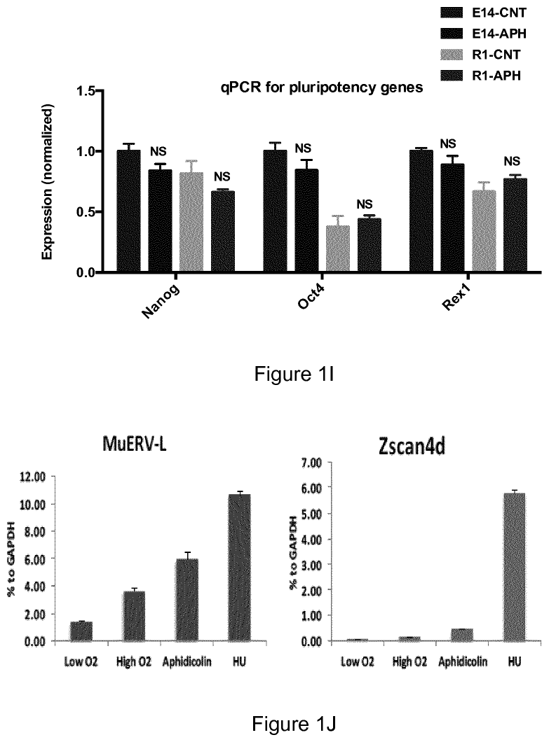

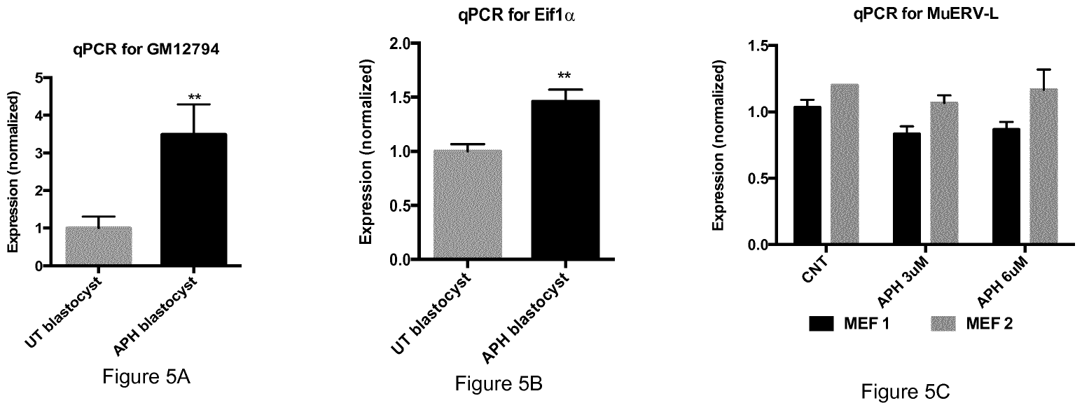

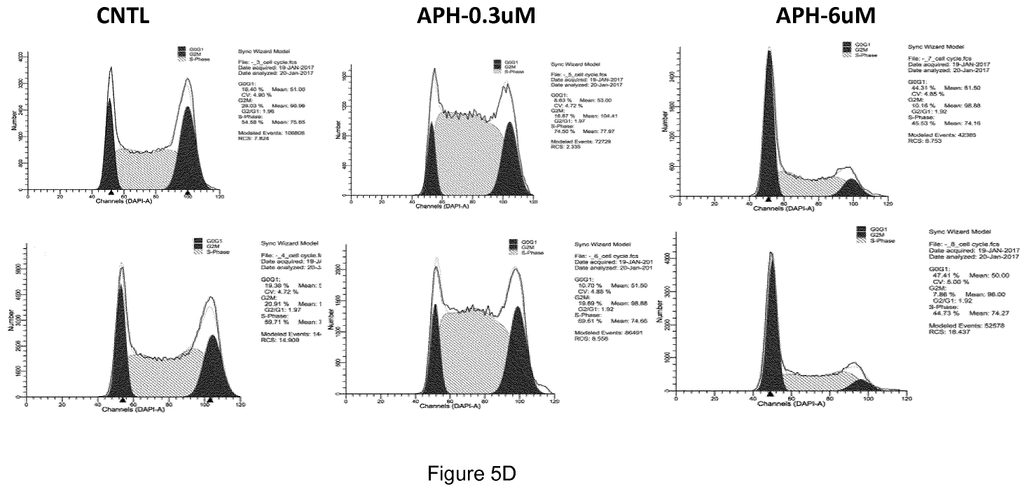

Maintenance of genome stability despite unlimited self-renewal is a unique feature of ESCs²⁶. Recent evidences have implicated that the key 2C-embryo-specific gene, Zscan4, is also expressed in ESCs where its transient burst was shown to play a critical role in maintaining genomic stability 5, 10. To evaluate the physiological relevance of these findings in vivo, we asked if forced induction of replication stress could activate the expression of key 2C-embryo specific genes in morula stage mouse embryos. To this aim, healthy synchronized morula-stage embryos were treated with polymerase inhibitor, aphidicolin and subjected to qPCR assay (FIG. 1a). Strikingly, induction of DNA damage response could activate several key 2C-embryo-specific genes including Zscan4, Tcstv3, GM12794 and Eif1a 2 (FIG. 1b-c; FIG. 5a,b).

In spite of the eminent genome stability, the presence of replication stress has been well documented in mESCs 11 and has been linked to high basal level of histone variant H2AX phosphorylation (γH2AX). As Zscan4 is periodically expressed in a fraction of ESCs, we tested whether its expression is associated with the accumulation of spontaneous replication stress. To test this hypothesis, we first examined the basal expression level of γH2AX and P-CHK1 as the indicators of DNA replication stress response in Zscan4-Emerald ESCs. Interestingly, flow cytometry analysis revealed a significant enrichment of γH2AX and P-CHK1 cells within Zscan4-Emerald positive ESC population with respect to Zscan4-Emerald negative ESC population (FIG. 1d-e).

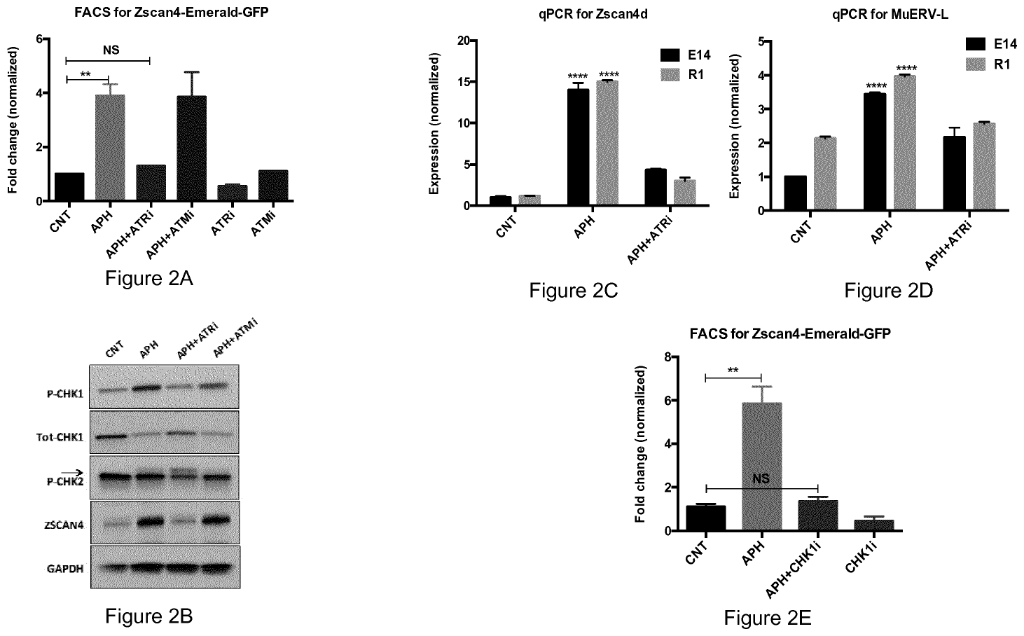

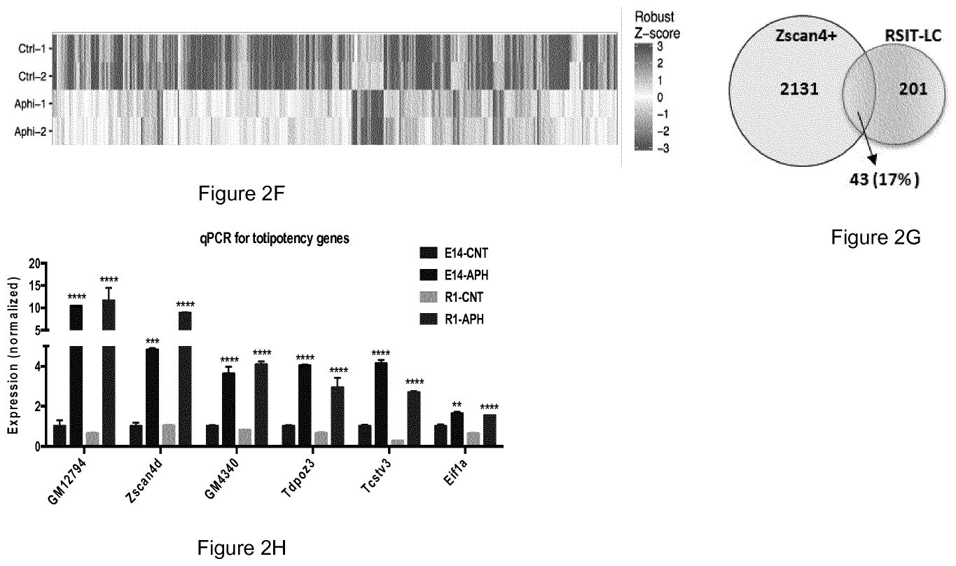

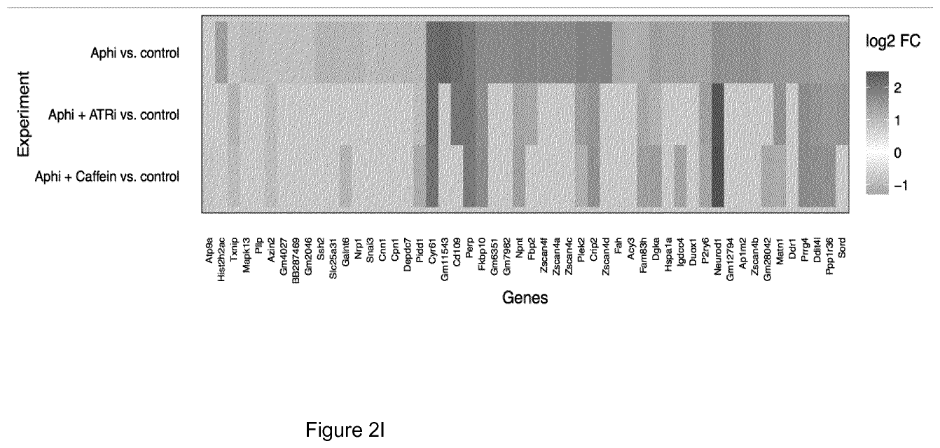

Due to the important role of MuERV-L-derived LTR elements in shaping the transcriptional signature of 2C-stage embryos 12, we next asked whether replication stress induced Zscan4 expression was accompanied by activation of MuERV-L retroelements. To this end, we exposed ESCs to a wide range of replication stress inducing agents including aphidicolin (APH), hydroxyurea (HU) and ultraviolet light (UV) (FIG. 1f-g; FIG. 2b) and 20% O₂ tension (FIG. 1j) 11. Strikingly, QPCR and FACS analysis showed that the expression of Zscan4 and MuERV-L are significantly upregulated upon all treatments in a time and dosage-dependent manner in various ESC lines with distinct genetic backgrounds (E14 and R1) (FIG. 1f-g; FIG. 5c). Overall, these findings indicate that spontaneous and induced replication stress lead to the activation of Zscan4 and 2C-stage related genes.

Two cell (2C)-like cells have been shown to reduce the expression of the pluripotency markers at the protein but not transcriptional level 1, 2. Thus, in order to understand if replication stress induced cells share such features with the 2C-like cells, we checked the expression of the canonical pluripotency markers upon APH treatment. In agreement with previous publications 1, 2, we found a mild downregulation of Nanog, Pou5f1 (Oct4) in Zscan4 positive cells upon immunostaining (FIG. 1h). However, no significant alterations in the expression of pluripotency related genes (e.g., Sox2, Nanog and Pou5f1) were observed at the transcriptional level (FIG. 1i).

Finally, we found that APH treatment do not alter the expression of main 2C-specific markers such as MuERV-L in mouse embryonic fibroblast (MEF) lines with distinct genetic backgrounds suggesting that the activation of 2C-like genes upon replication stress could be cell type specific (FIG. 5d). These results overall suggest that transition to Zscan4 positive cells in ESC culture is regulated through DNA replication stress response.

Next, to gain further insight into the mechanisms through which replication stress response could contribute to the reactivation of 2C-like specific genes in ESC culture, we asked if suppression of DNA damage response pathway (DDR) could restrain the reactivation of main 2C-like genes under normal culture condition. Thus, we inhibited DDR by addition of specific ATM and ATR inhibitors (KU-55933 and VE 822, respectively) in Zscan4-Emerald ES cells. Flow cytometry analysis revealed that the addition of ATRi could markedly reduce the number of Zscan4-Emerald positive cells however, treatment with ATM inhibitor did not change the number of the Zscan4 positive population in the dish significantly (FIG. 2a).

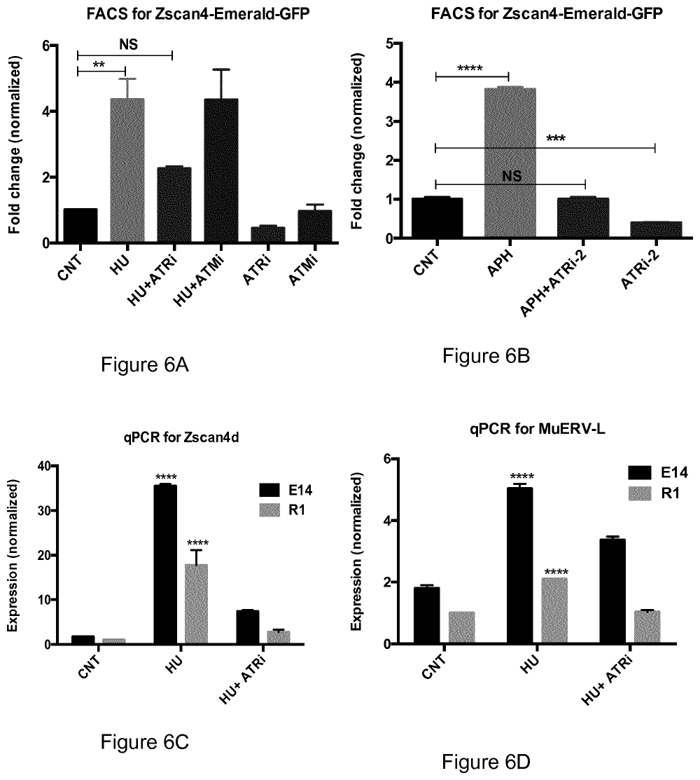

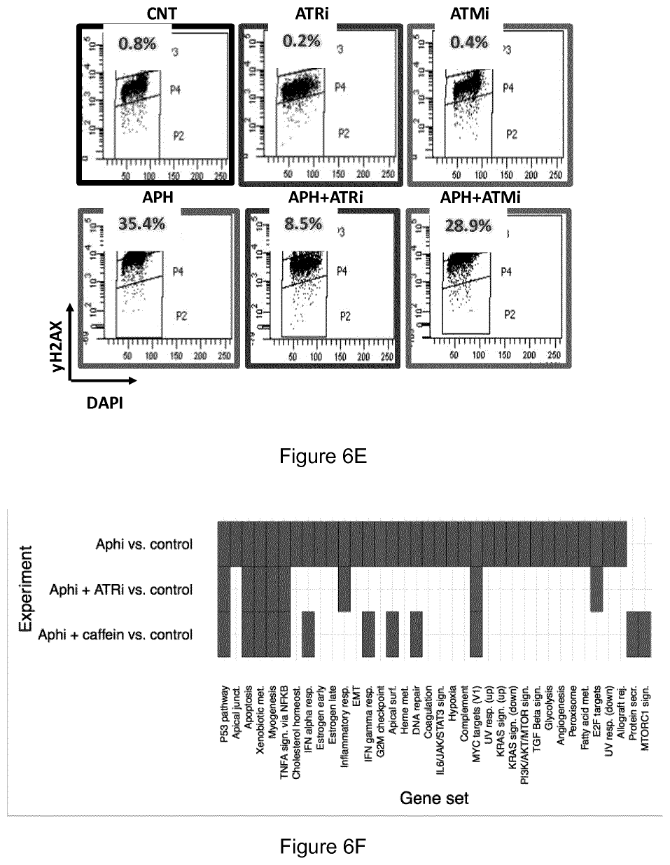

Next, we asked whether inhibition of DDR could revert the transcriptional activation of MuERV-L and Zscan4d under replication stress inducing condition to uncover which specific pathway is mostly involved in replication stress induced 2C-like genes activation. To this aim, Zscan4-Emerald ES cells were treated with specific ATM and ATR inhibitors upon APH and HU treatment. While the immunoblot results confirmed the robust and highly specific inhibition of ATM and ATR pathway upon replication stress (shown by the phosphorylation status of CHK1, CHK2), flow cytometry analysis and immunoblot revealed that ATR inhibitor could robustly suppress the elevation of 2C-like specific genes whereas ATM inhibition could not alter the elevated expression of Zscan4 upon APH and HU treatment (FIG. 2a,b; FIG. 6a). These results were further validated on two established ESC lines (E14 and R1) through qPCR assay for Zscan4 and MuERV-L genes (FIG. 2c,d; FIG. 5-d). Of note, similar results were also obtained upon CHK1 and ATR inhibition through different inhibitors suggesting that ATR mediated response triggers the activation of key 2C-like genes in ESC culture (FIG. 2e; FIG. 5b). Finally, the number of Zscan4 positive population both under normal condition but also upon APH treatment, noticeably correlated with the number of γH2AX positive cells in line with recent studies where the bona fide replication stress and high level of γH2AX in unperturbed ESCs were found to be dependent on ATR but not ATM 11 (FIG. 5e).

Next, to understand if activation of ATR dependent response could result in global transcriptional alterations similar to that of 2 cell-stage embryos, we performed whole gene expression profiling on two established mESC lines namely E14 and R1 upon APH treatment. Analysis of differentially expressed genes (DEGs) (False discovery rate (FDR)-adjusted P-value cutoff) identified 193 upregulated genes with more than two-fold changes in gene expression upon APH treatment, and only 21 downregulated genes which is in agreement with general openness of the chromatin in totipotent state 1 (FIG. 2f). To understand how many of the identified DEGs overlap with those specifically expressed in 2C-like cells we compared our DEGs with those of previously published datasets 2, 10, 13. Through such comparison, we found that 17% (43 out of 201) of DEGs overlap with totipotency specific genes among which qPCR results validated significant upregulation of main 2C-like related genes including GM4340, Gm12794, Zscan4 genes (Zscan4b and Zscan4d), Zfp352, Zfp750, Tdpoz genes (Tdpozl and Tdpoz3), Eif1a and Tmem19 (FIG. 2g,h).

Next, we aimed to dissect the role of ATR on the global transcriptional reversion of replication stress induced 2C-specific genes. While the geneset enrichment analysis confirmed a robust silencing of DNA repair signaling pathway upon addition of specific ATR inhibitors and Caffeine (FIG. 5f). Strikingly, the heatmap analysis identified considerable portion 72% (31 out of 43) of genes which has reverted their expression level upon addition of ATR inhibitor and Caffeine, respectively (FIG. 2i).

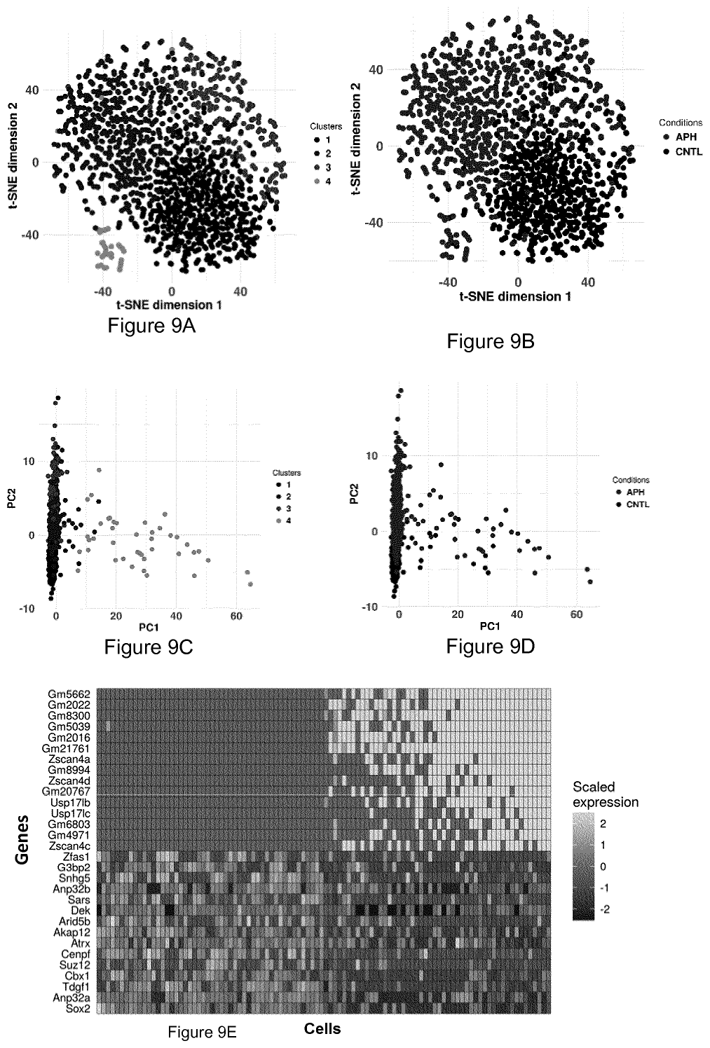

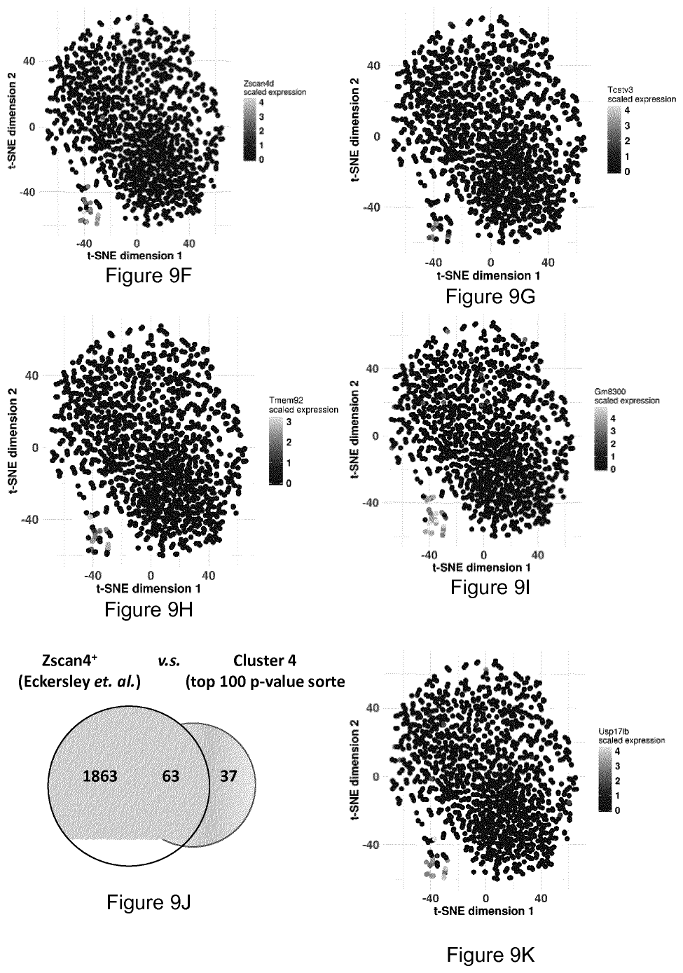

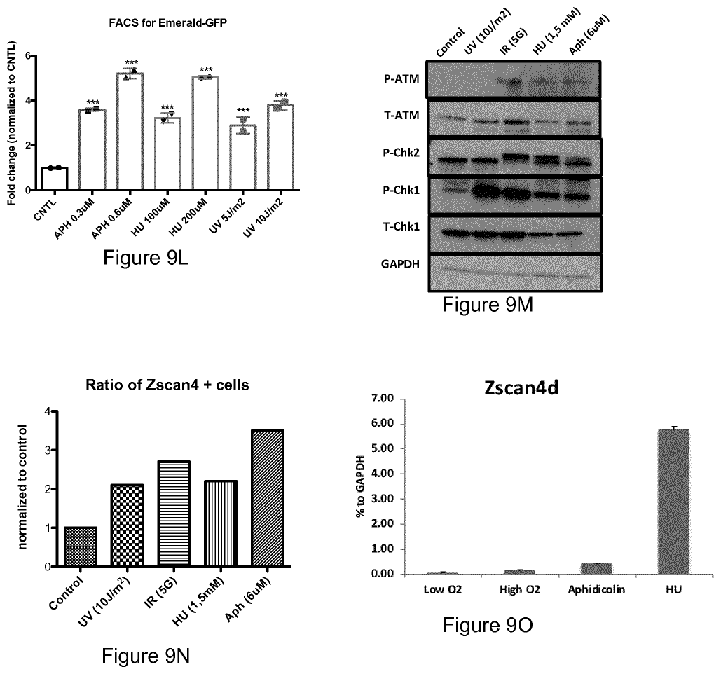

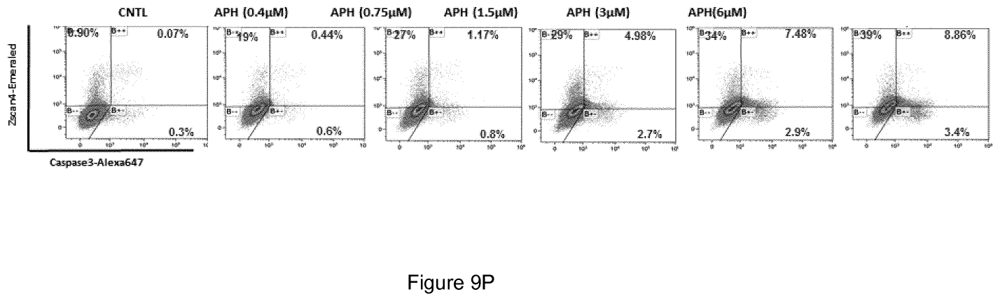

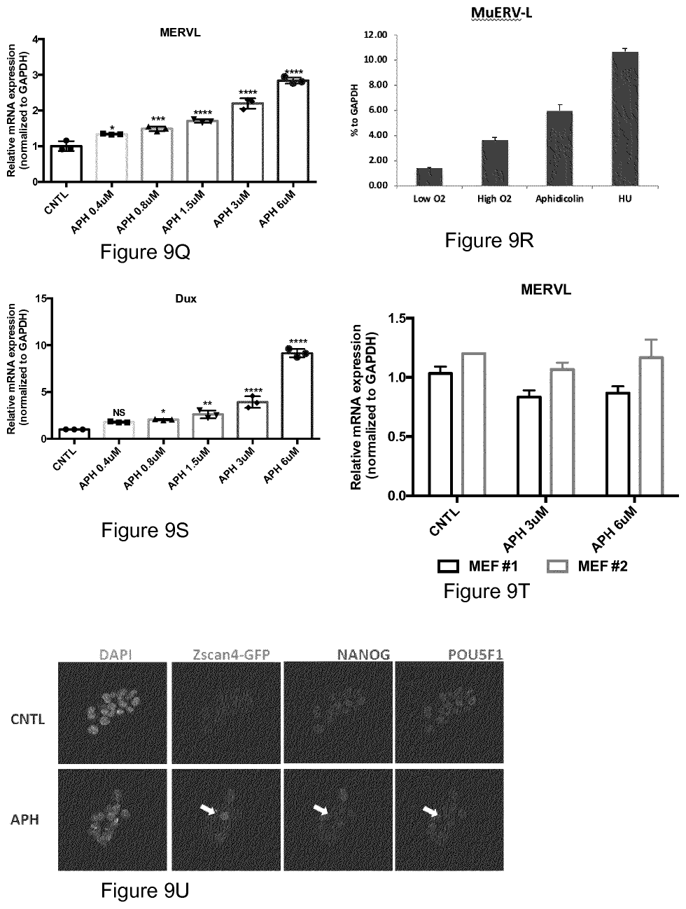

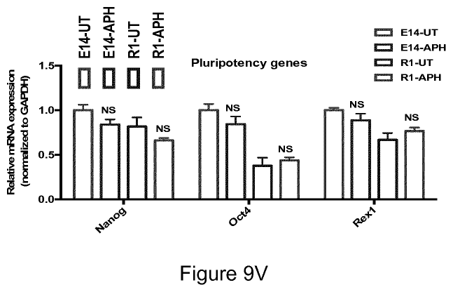

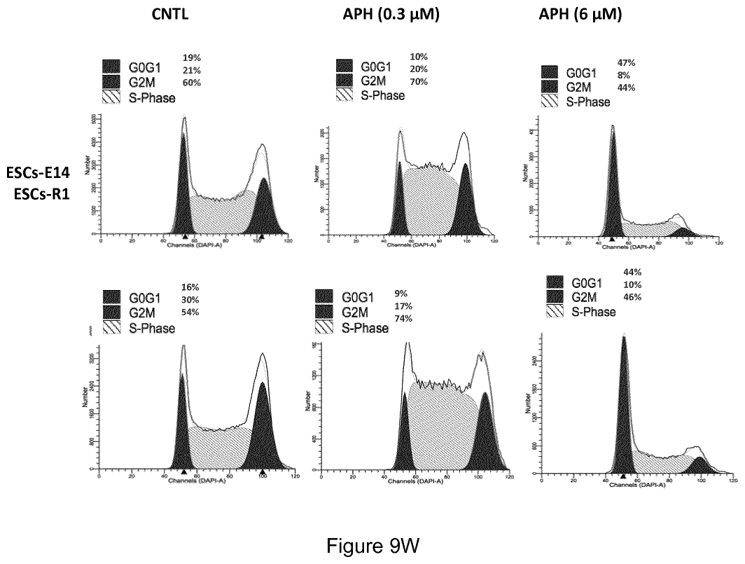

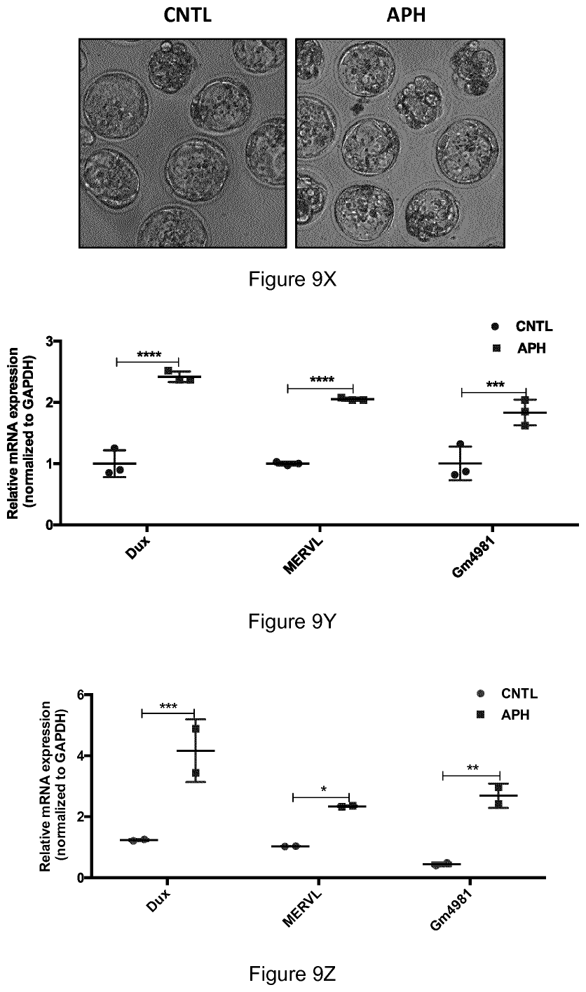

To understand how ESCs coordinate these functions, we asked how ESCs respond to RS at the single cell level. To this end, we performed single cell transcriptional profiling²⁷ on E14 mouse ESCs cultivated in Leukemia Inhibitory Factor (LIF) plus MEK and GSK inhibitors (2i) upon treatment with aphidicolin (APH), a reversible inhibitor of DNA polymerase that induces RS by stalling replication fork progression²⁸. Unsupervised clustering analysis²⁷ identified four distinct clusters of cells (FIG. 9a); three of which largely overlapped with control (CNTL) and APH treated conditions (FIG. 9a, cluster 1, 2 and 3), along with the emergence of a small cell population (FIG. 9a, cluster 4) that was significantly increased upon RS (FIG. 9b). Notably, Principal Component (PC) 1 clearly separated this small population from the rest of the cells (FIG. 9c,d) Extended data FIG. 1a, b). Remarkably, the analysis of differentially expressed genes (DEGs) identified a significant enrichment of 2C-like cell specific genes in the newly emerging population (cluster 4), including Eif1a-like genes (Gm5662, Gm4027, Gm2016, and Gm8300), Tcstv3, and Zscan4 genes (Zscan4a-Zscan4d), (FIG. 9e-k), whose transcriptional burst has been shown to play a critical role in maintaining ESCs genome stability^{5,29,30}. To understand whether the increase in the number of 2C-like cells was due to the specific role of APH as a RS inducing agent, we exposed pZscan4-Emerald ESCs (generated by the stable introduction of Emerald-GFP reporter under the Zscan4c promoter⁵) to a range of RS inducing agents, including APH, hydroxyurea (HU), 20% O₂ tension, irradiation (IR) and ultraviolet light (UV)²⁸. The qPCR and Fluorescence Activated Cell Sorting (FACS) analysis confirmed a significant increase in the number of Emerald-GFP positive (Em⁺) ESCs across all treated conditions in a dose-dependent manner (FIG. 9l-o). Although APH, mostly at high concentration, induced mild cell apoptosis, the majority of Em⁺ cells were not co-stained with apoptosis marker CASPASE-3, suggesting that emergence of ZSCAN4 positive (Z+) cells upon APH treatment was not due to activation of the apoptosis pathway (FIG. 9p).

Due to the key role of Dux and MERVL-derived long terminal repeat (LTR) elements in shaping the transcriptional signature of 2C-stage embryos and 2C-like cells^{28-10,31}, we next asked whether RS-induced expression of Zscan4 genes was accompanied by activation of these retroelements. The qPCR results revealed a linear correlation between concentration of RS inducing agents and the expression of key 2C-like markers, Dux and MERVL (FIG. 9q-s). However, induction of RS did not affect the expression of main 2C-like specific markers such as MERVL (FIG. 9t) and Zscan4 (no expression was detected by qPCR) in two mouse embryonic fibroblast (MEF) lines with distinct genetic backgrounds (C57BL/6J and 129P2/OlaHsd), suggesting involvement of alternative repressive mechanisms that suppress the activation of 2C-like related pathway in more differentiated cells.

2C-like cells reduce the expression of pluripotency markers at protein but not transcriptional level^{1,2,31}. Thus, to understand whether RS-induced cells share such features with the 2C-like cells, we monitored the expression of the canonical pluripotency markers upon APH treatment. In agreement with previous findings^{1,31}, we detected downregulation of NANOG and POU5F1 (also known as OCT4) proteins in Z⁺ cells by immunostaining (FIG. 9u). However, no significant alterations in the expression of pluripotency related genes (e.g., Nanog, Pou5f1 and Rex1) were observed at the transcriptional level (FIG. 9v).

The Z+/MERVL cells were reported to be present in all phases of the cell cycle albeit with higher percentage in G2/M phase¹³. Of note, the cell cycle analysis on pZscan4-Emerald ESCs confirmed that APH treatment neither at low (0.3 μM) nor at high (6 μM) concentrations could increase G2/M population in culture (FIG. 9w), indicating that augmentation of Z⁺ population upon APH treatment was not due to cell cycle arrest in G2/M. This is consistent with previous work showing that G2/M arrest by nocodazole is not sufficient to trigger Zscan4 expression³². Finally, to evaluate the physiological relevance of these findings in vivo, we asked whether transient induction of RS could activate the expression of key 2C embryo specific genes in mouse embryos. To this end, morula-stage embryos were treated with APH, and subsequently the synchronized embryos were selected and subjected to qPCR (FIG. 9x). Strikingly, induction of RS activated the expression of several key 2C embryo specific markers, including Dux, MERVL and Gm4981 in mouse embryos (FIG. 9y,z).

Overall, these findings indicate that RS leads to the activation of 2C embryo specific genes in ESCs and mouse embryos.

### Example 3: Activation of Endogenous DNA Damage Pathways is Responsible for the Emergence of 2C-Like Cells Under Normal Culture Condition

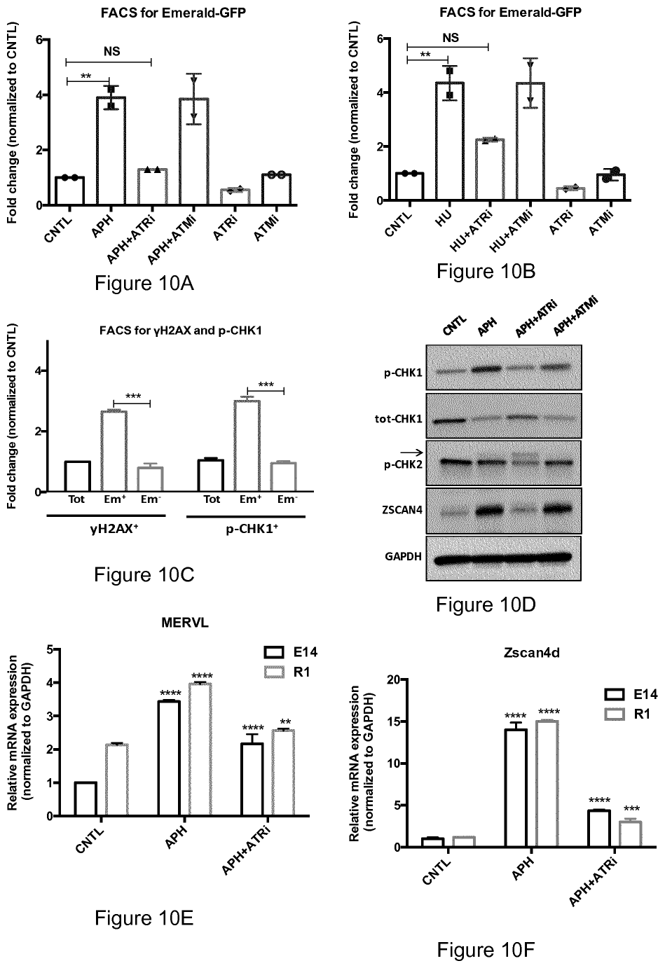

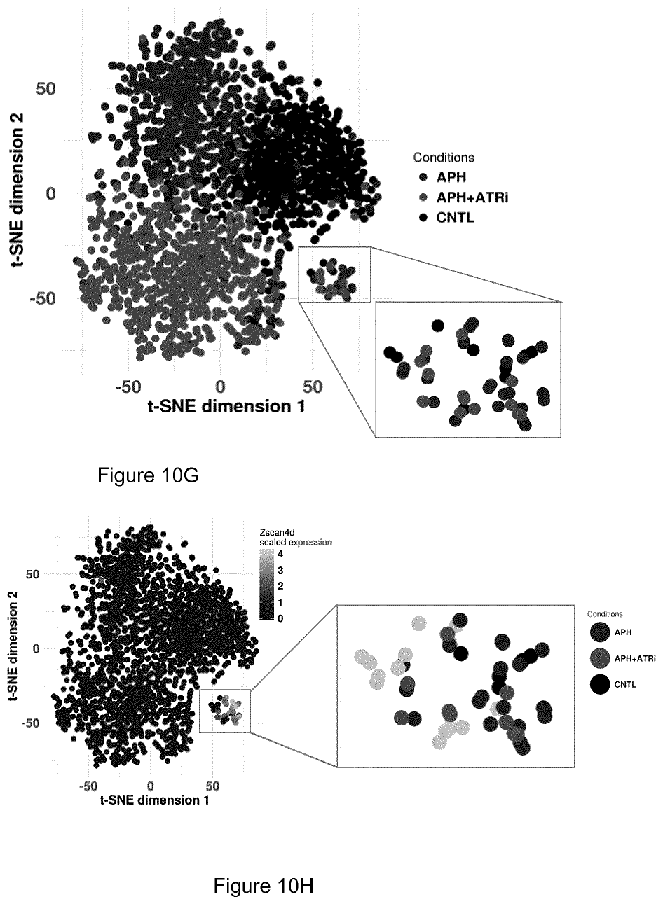

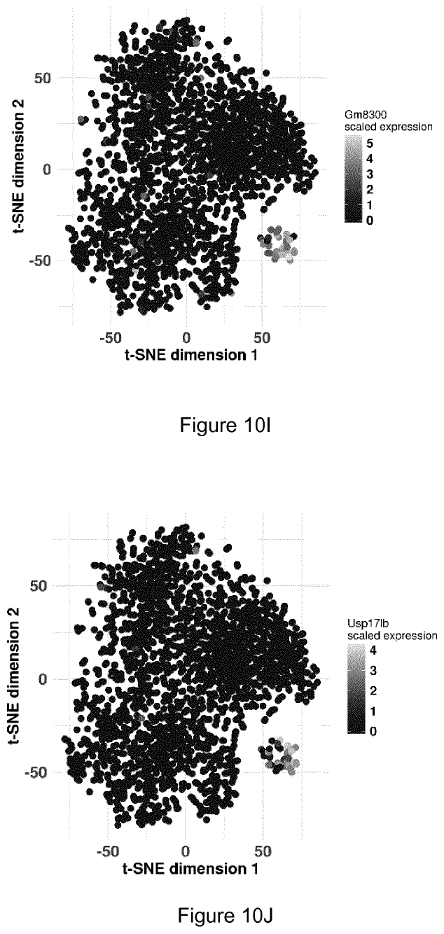

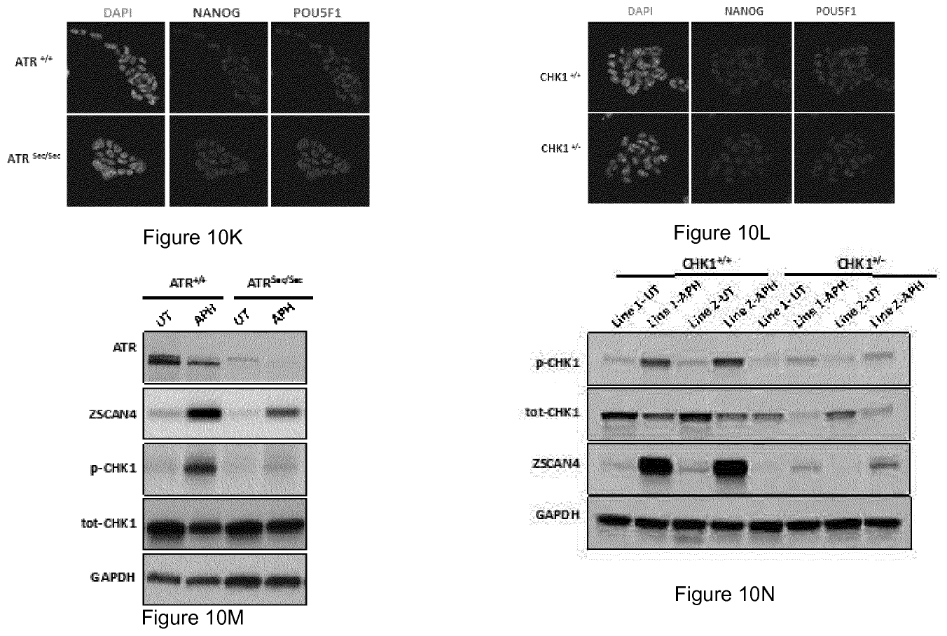

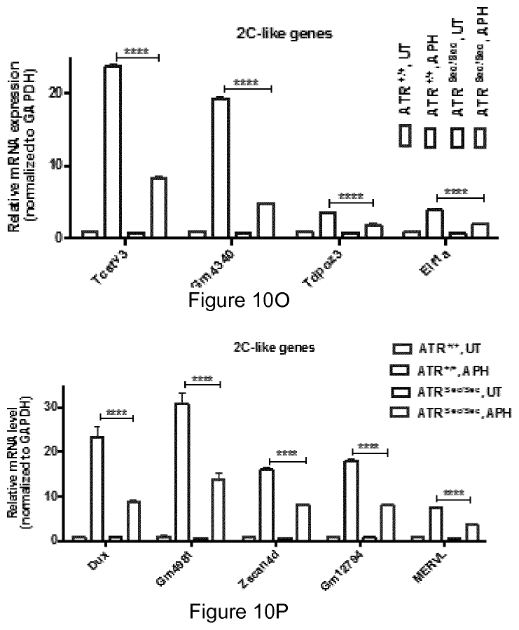

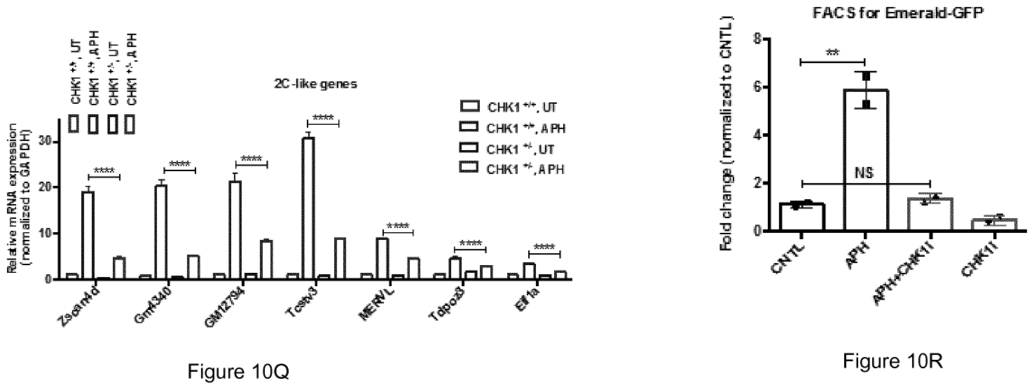

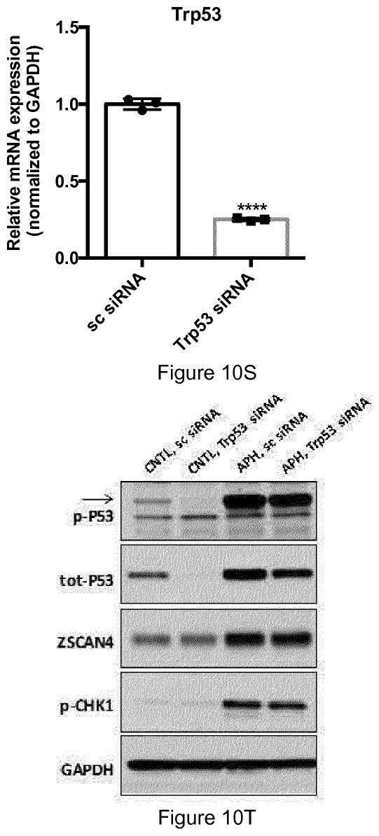

Next, to gain further insight into the mechanisms through which RS response (RSR) could contribute to the emergence of 2C-like cells in ESCs culture, we asked whether activation of the endogenous DNA damage response pathways (DDR) is responsible for the emergence of 2C-like cells under normal culture condition. Thus, we inhibited DDR by treating pZscan4-Emerald ESCs with specific ATM and ATR inhibitors (ATMi, KU-55933 and ATRi, VE 822, respectively). FACS analysis revealed a marked reduction in the number of Em⁺ cells upon ATR inhibition. However, treatment with ATMi did not significantly reduce the fraction of Em⁺ cells in culture (FIG. 10a,b). In addition, FACS analysis revealed a significant enrichment of γH2AX and p-CHK1 (indicators of RS) populations within Em⁺ ESCs (FIG. 10c), suggesting that the transient activation of ZSCAN4 is linked to the presence of endogenous RS in 2C-like cells. Consistent with these results, inhibition of ATR but not ATM activity could robustly revert the expression of RS-induced (HU and APH-induced) 2C-like specific markers, ZSCAN4 and MERVL (FIGS. 10a,b and d). These results were further validated on E14 and R1 ESC lines through qPCR assay for Zscan4 and MERVL (FIG. 10e,f).

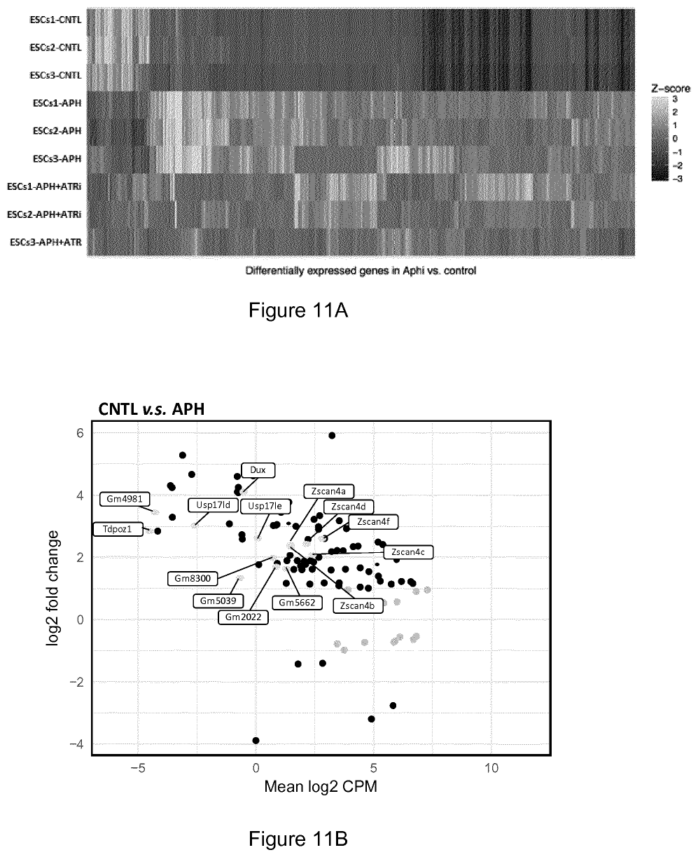

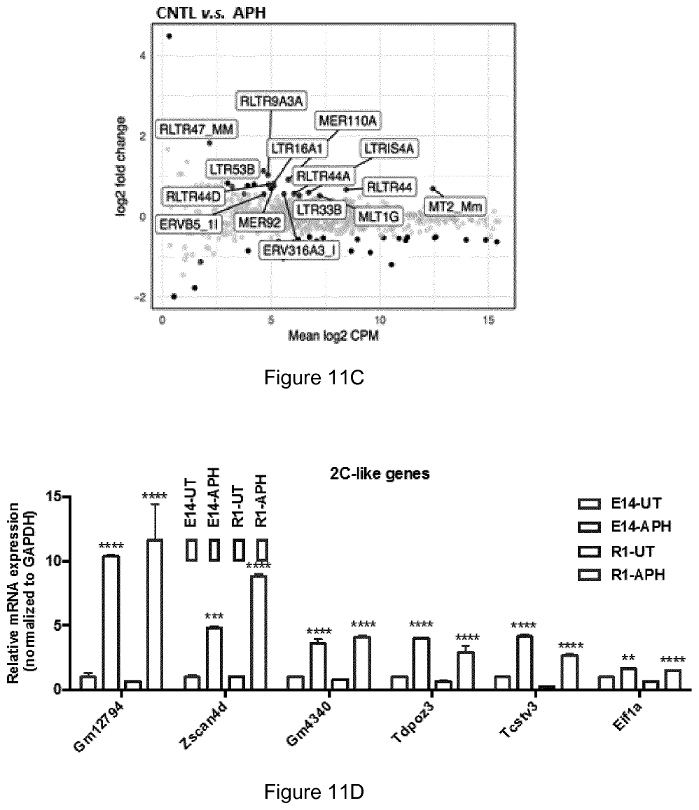

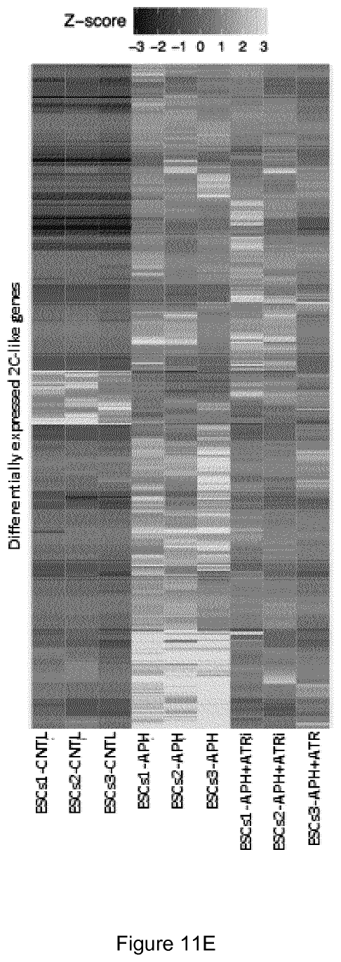

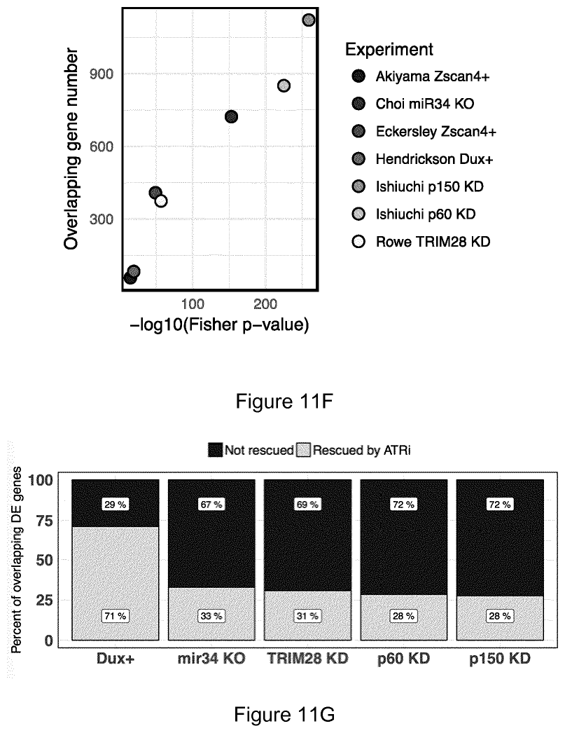

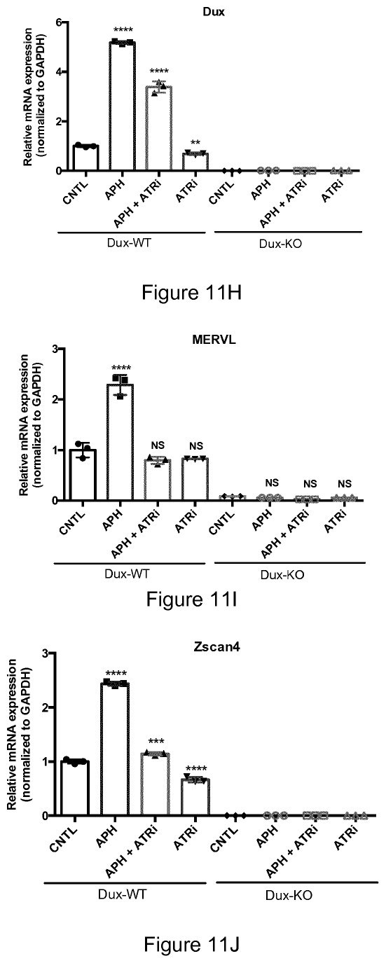

In agreement with our findings, single cell transcriptional profiling revealed that the number of RS-induced cells in the newly emerging cluster was significantly reduced upon ATR inhibition (FIG. 10g). In addition, suppression of ATR activity by ATRi also led to a marked reduction in the number APH-induced 2C-like gene expressing cells (e.g., Zscan4d⁺, Gm8300⁺, and Usp14ib⁺) (FIG. 11h-k).

### Example 4: ATR and CHK1-Mediated RS Response Triggers Activation of Key 2C-Like Genes in ESCs

Next, to exclude any unspecific inhibitory effect of ATR and CHK1 inhibitors on the transcriptional repression of 2C-like genes, we aimed to generate ATR Seckel and CHK1 heterozygous ESCs from previously reported mice models 14, 15 as the complete ablation of ATR and CHK1 causes embryonic lethality. To this aim, two heterozygous mouse for ATR Seckel and CHK1 allele were crossed and ESC were isolated at embryonic day 3.5 (E3.5). On the basis of genotyping results, two homozygous ATR Seckel and two heterozygous CHK1 mESC were used for further characterization (FIG. 6a,b).

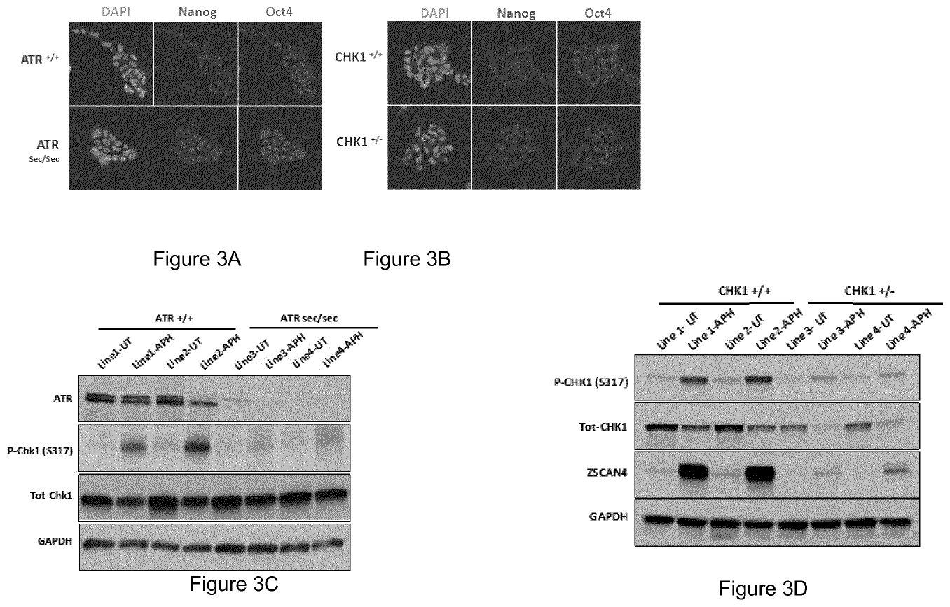

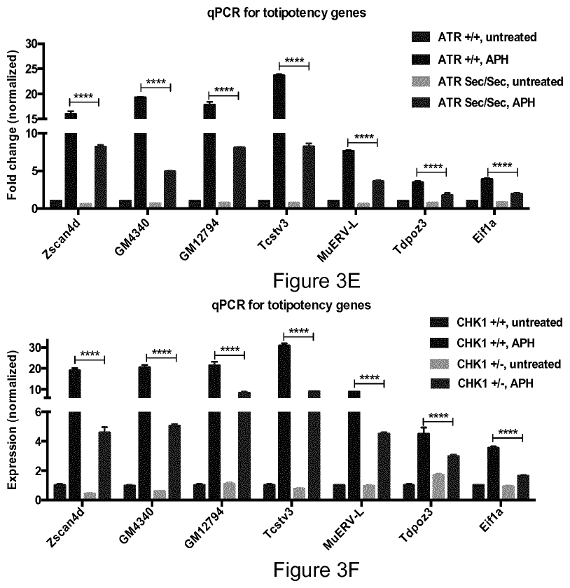

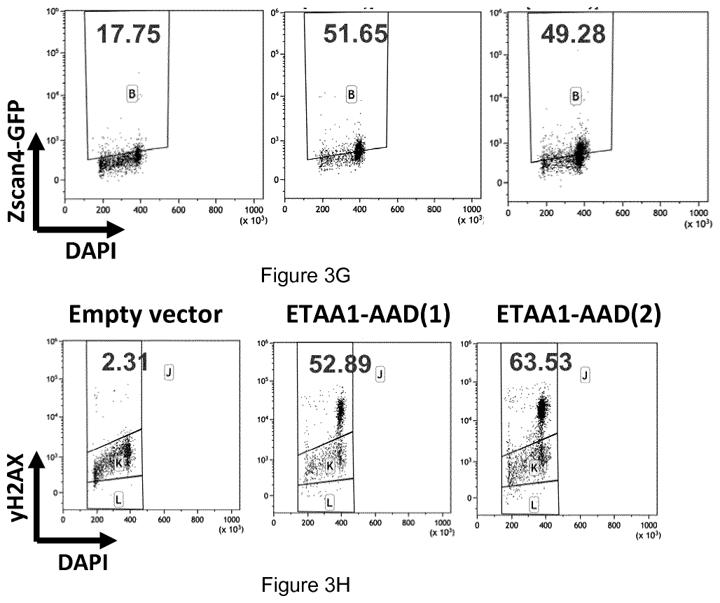

Although immunoblot results confirmed that the level of ATR and CHK1 protein is severely reduced in comparison to the wild type control (FIG. 3c,d), no significant alterations in the expression of key pluripotency genes were observed with respect to the wild type control by immunofluorescent. (FIG. 3a,b) This suggest that though introduced mutation reduces the abundance of ATR to almost undetectable, yet the remaining protein is sufficient for viability and maintenance of mESC.

Importantly, the qPCR results in two ATR Seckel and two control lines on a large panel of 2C-like specific genes validated our previous data. Of note, similar to inhibitory impact of ATR inhibitors on expression of 2C-like genes in ESC, APH treatment on ATR Seckel ESC led only to a milder increase of 2C-like genes expression unlike normal wild type ESC (FIG. 3e; FIG. 6c). In line with these finding, immunoblot results showed that the level of ATR protein in ATR Seckel ESCs was markedly decreased in comparison to WT controls. Moreover, upon APH treatment the level of phospho-Chk1 protein (S345 and S317) was found to be significantly lower in ATR Seckel ESCs with respect to wild type controls. As expected, similar results were obtained in CHK1 heterozygous ESC (FIG. 3e,f; FIG. 6,d), which overall, exhibit a robust association between activation of ATR and expression of 2C-like specific genes.

To further validate these findings, we derived ATR-deficient Seckel (ATR^{Sec/Sec}) and CHK1^{+/-} ESCs from previously reported mouse models^{33,34} as the complete ablation of ATR or CHK1 causes embryonic lethality in mice^{34,35}. To this end, ESC lines were established in culture from pre-implantation embryos obtained from crosses between heterozygous mice either for ATR Seckel or for CHK1 KO alleles. On the basis of genotyping results, two homozygous ATR Seckel and two heterozygous CHK1 ESC lines were characterized for further investigations (FIG. 10l-o). Although immunoblot results confirmed that the level of ATR and CHK1 proteins was severely reduced in comparison to the wild type (WT) ESCs (FIG. 10m, o), no significant alterations in the expression of key pluripotency genes were observed in ATR^{Sec/Sec} or CHK1′ ESCs compared to WT ESCs (FIG. 10l,n). Noticeably, similar to the inhibitory impact of ATRi on the expression of 2C-like genes, APH treatment on ATR^{Sec/Sec} ESCs led only to a mild increase in the expression of 2C-like genes unlike normal WT ESCs (FIG. 10m,p,q). As expected, similar results were obtained with CHK1^{+/-} ESCs and upon treatment with CHK1 inhibitor (CHK1i) (FIG. 10o,r,s) but not upon p53 knockdown (KD), suggesting that activation of 2C-like pathway does not require the known mediator of DDR, p53. (FIG. 10t, u).

Overall, these data demonstrate that an ATR and CHK1-stimulated response to endogenous and induced RS regulates the activation of 2C-like specific genes.

Recent evidence point to the activation of the ATR kinase by the RPA-binding protein, Ewing's tumor-associated antigen 1 (ETAA1) 16. Importantly, several lines of evidence suggested that, the γH2AX positivity caused by ATR activating domain (ETAA1-AAD) overexpression is not a consequence of DNA breakage 16, 17. Hence, in order to exclude the possibility of the activation of 2C-like gene due to physical damage of DNA and not due to ATR activation, we aimed to activate ATR in DNA damage free context through ETAA1-ATR activating domain (AAD) overexpression. To this aim, Zscan4-Emeraled ESCs were infected by two independent ETAA1-ADD expressing lentiviruses. FACS analysis on mCherry positive infected cells, confirmed activation of ATR pathway (i.e., increase in the number of γH2AX positive cell) upon infection with ETAA1-AAD lentivirus with respect to the empty vector (EV) infected cells (FIG. 3g,h). Strikingly, activation of ATR was accompanied by up to 3-fold increase in the number of Zscan4-positive population in ESC culture (FIG. 3g,h).

These data collectively further consolidate that activation of ATR, even in the absence of replication stress, could trigger the global reactivation of 2C-like gene network and that ATR inhibition exerts a robust suppressive impact on such transcriptional activation.

### Example 5: ETAA1-Mediated Direct Activation of ATR Induces 2C-Like Cells in a RS-Free Context

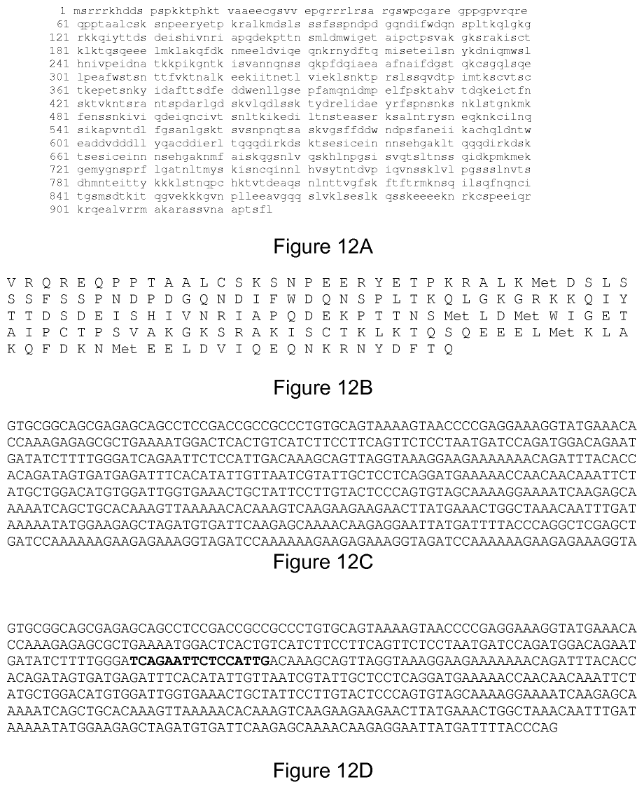

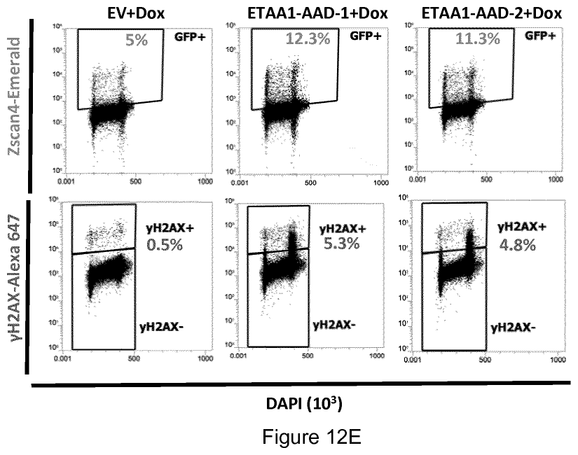

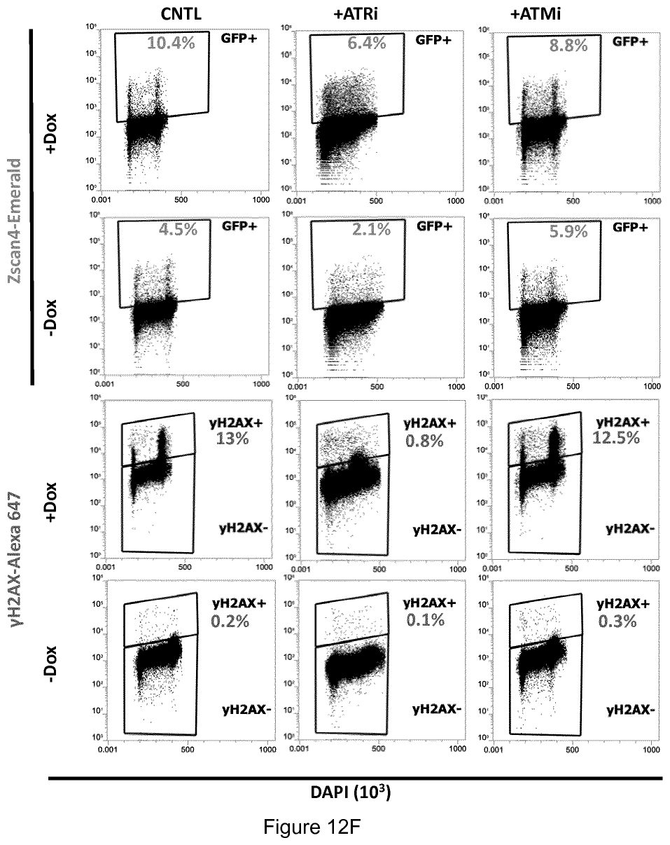

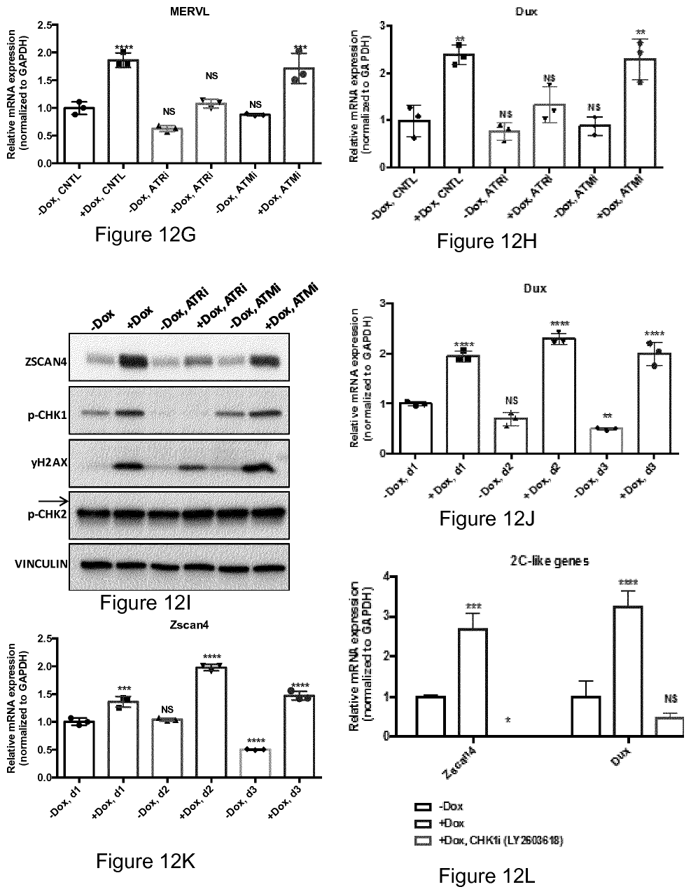

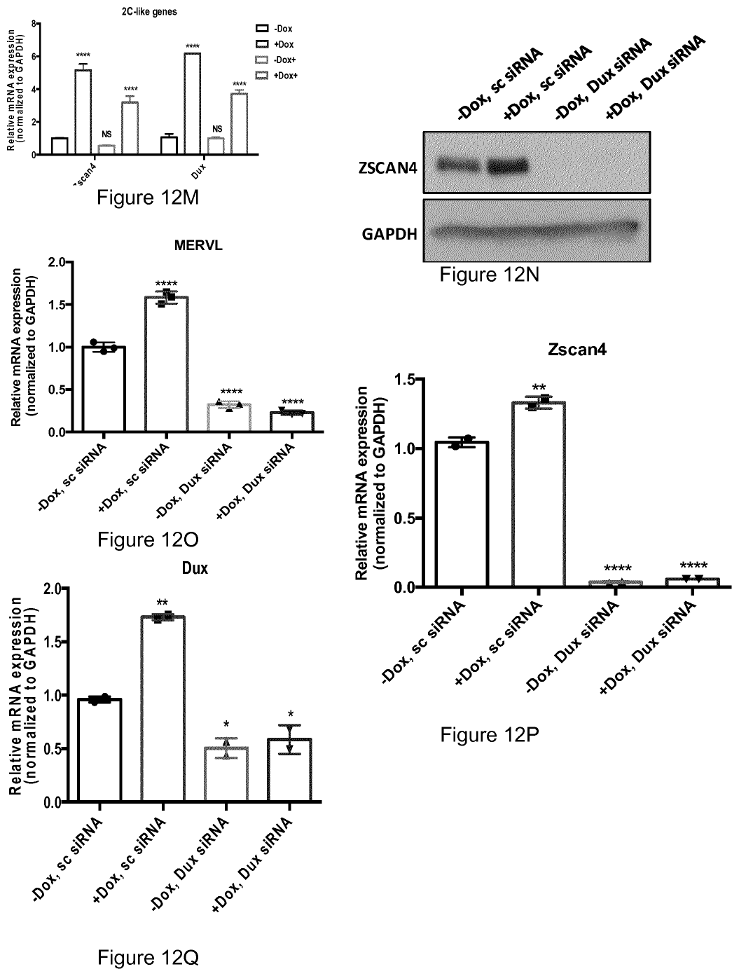

Recent evidence showed that ATR kinase can be directly activated by the RPA-binding protein, Ewing's tumor-associated antigen 1 (ETAA1) in the absence of RS^{38,39}. Hence, to confirm that the activation of 2C-like genes is due to ATR-mediated response but not the physical damage to DNA, we aimed to activate ATR in a DNA damage-free context through overexpression of ETAA1-ATR activating domain (ETAA1-AAD) in ESCs. To this end, pZscan4-Emerald ESCs were infected with two independent lentiviruses generated from two different clones of ETAA1-expressing lentivectors in which ETAA1-ADD was expressed under the control of a doxycycline (Dox) inducible promoter, and subsequently selected against puromycin and neomycin for two weeks (FIG. 12a,b). As expected, FACS analysis, confirmed the activation of the ATR pathway as shown by the increase in the number of γH2AX cells upon Dox-inducible expression of ETAA1-AAD in infected ESCs (Dox-iETAA1 ESCs) compared to ESCs infected with empty vector (EV) (FIG. 12c). Strikingly, ETAA1-stimulated activation of ATR was accompanied by a significant increase of the Z⁺ population in ESCs culture and transcriptional activation of Zscan4, Dux and MERVL (FIG. 12d-i). As expected, this transcriptional activation was fully abolished upon ATR and CHK1 inhibition (FIG. 12d-k).

Importantly, several lines of evidence demonstrated that the γH2AX positivity caused by the overexpression of ETAA1 was not a consequence of DNA breakage^{38,39}. We also confirmed that ATR activation through ETAA1 expression leads to the phosphorylation of H2AX and CHK1 but not CHK2 in ESCs (FIG. 12d, g). Moreover, while inhibition of ATR activity reduced the level of γH2AX upon ETAA1-mediated direct activation of ATR, inhibition of ATM activity did not exert any noticeable effect (FIG. 12d, g). Finally, we demonstrated that activation of canonical 2C-like genes upon ETAA1-mediated ATR activation is DUX dependent, as Dux KD completely prevented ETAA1-mediated expression of MERVL and ZSCAN4. (FIG. 12i-o).

These results collectively indicate that DNA damage-free ETAA1-stimulated ATR activation in ESCs is sufficient to activate 2C-like genes through DUX induction.

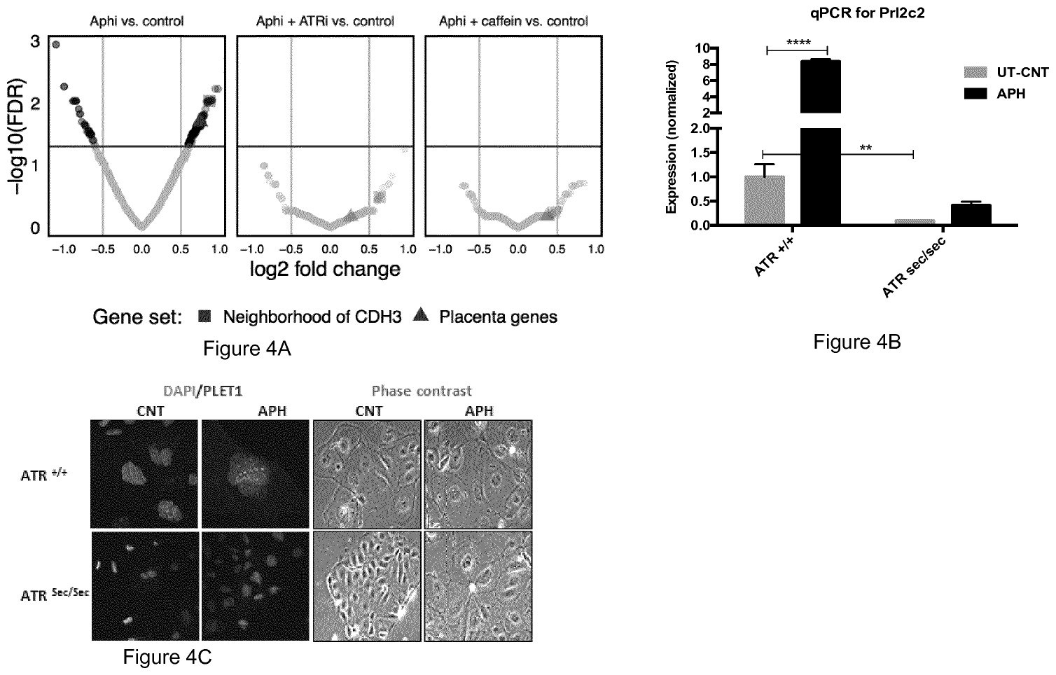

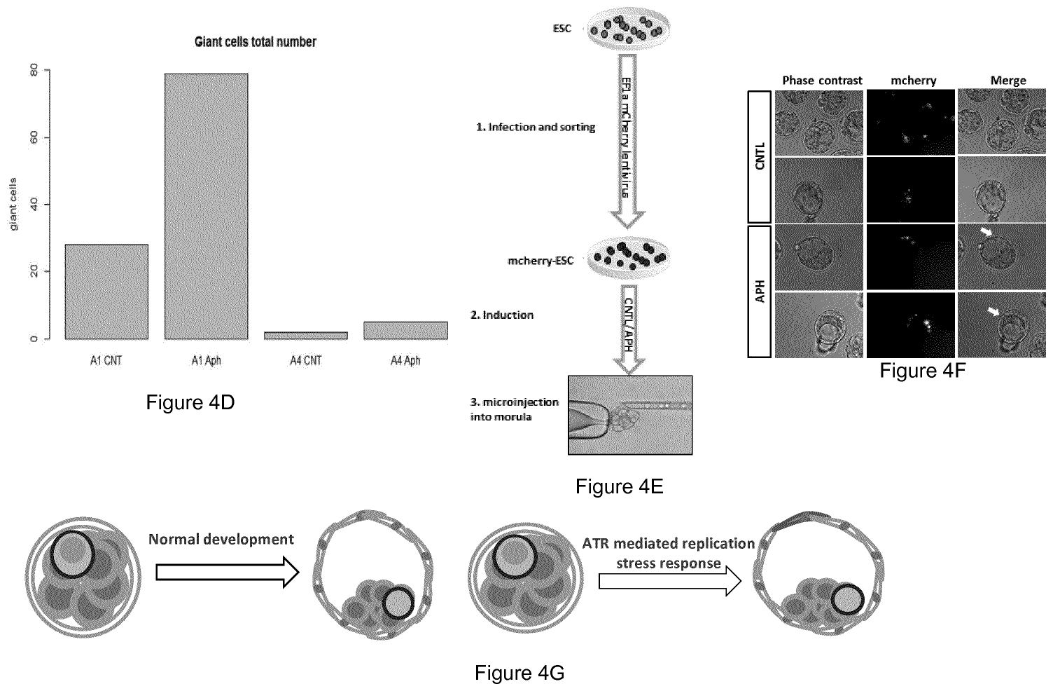

Our results so far indicated that ATR activated cells exhibit similar transcriptional profile and characteristics of 2-cell-stage embryos. Strikingly, computational gene-sets analysis of APH vs CNTL through molecular signature database (MSigDB), identified a strong and highly significant enrichment in two placenta-related gene categories which are robustly suppressed upon ATR inhibition by specific ATR inhibitor and Caffeine (FC and p-value) (FIG. 4a).

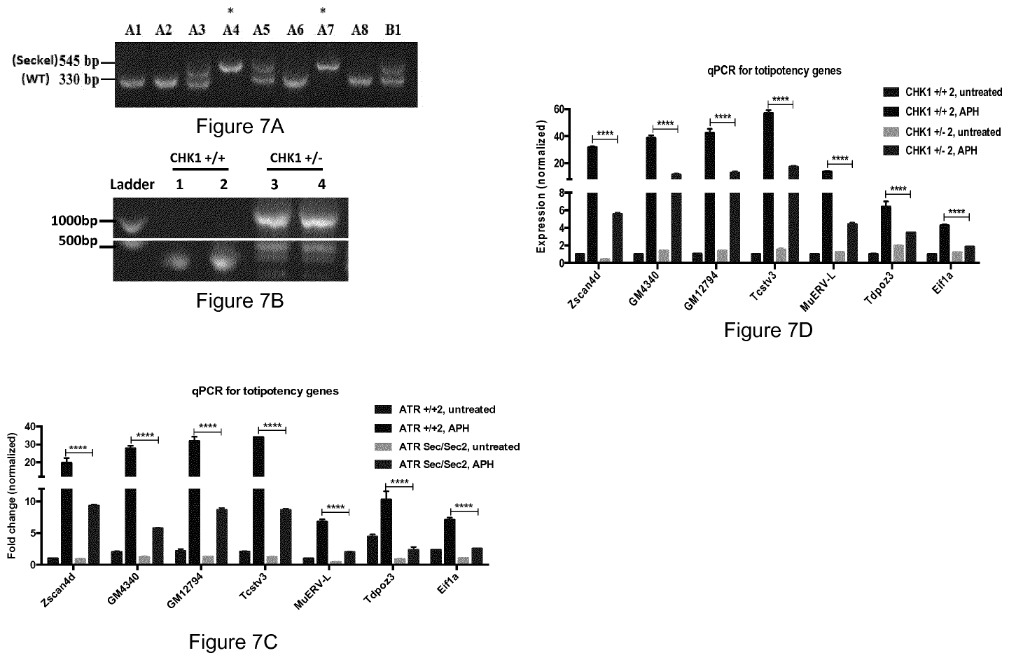

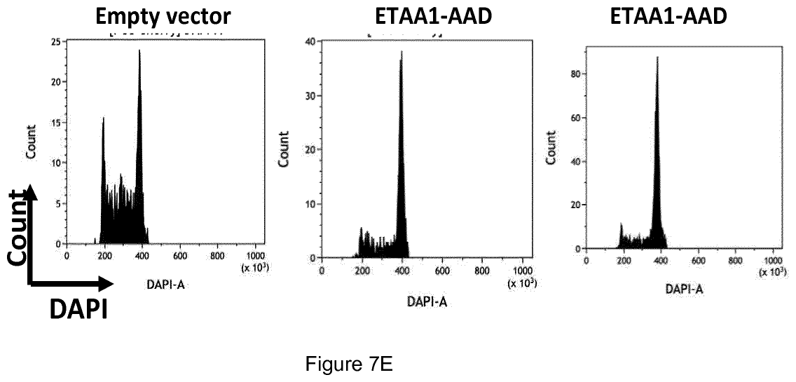

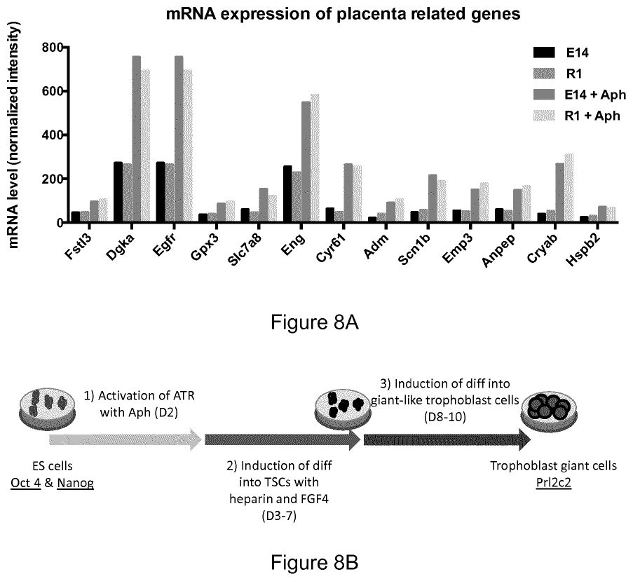

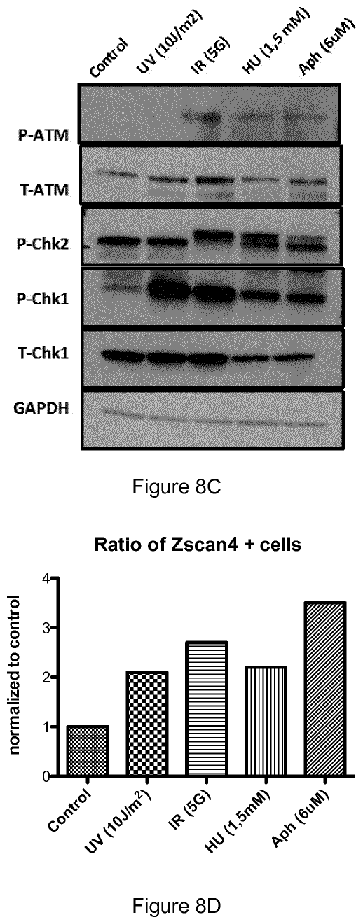

In addition, whole transcriptional profiling results also revealed the activation of several key placenta related genes upon replication stress in ESC including; Dgka, Egfr, Slc7a8, Eng, Cyr61, Adm, Scn1b, Emp3, Anpep, Cryab and Hspb2, among which some (e.g., Dgka, Eng and Egfr) are interestingly involved in exosome formation and angiogenesis, the critical pathways that are potentially exploited by placenta to gain migratory, invasive and immune suppressive features 18 (FIG. 7).

These findings led us to ask whether ATR activated ESC also gained expended development potency to be differentiated to extra-embryonic tissues. To test this hypothesis, both ATR Seckel and wild type ESC cells (upon treatment with APH) were differentiated in vitro toward trophoblast-like stem cells for three days followed by terminal differentiation to giant trophoblast-like cells upon withdrawal of FGF4 and heparin for 3 additional days (FIG. 7b) 19.

The qPCR results for the giant trophoblast cell specific marker, Prl2c2 revealed the highest expression in ATR activated condition while ATR Seckel cells could not significantly upregulate such a marker in the absence of APH (FIG. 4b). However, the level of Prl2c2 were found to have four-fold increase in ATR activated ATR Seckel cells with respect to the non-treated ATR Seckel cells (FIG. 4b). These results were also consistent with the number of giant trophoblast-like cell in four indicated conditions where APH treatment could increase the number of giant cells more than three folds (FIG. 4c,d). Of note, giant trophoblast-like cells were found to have a higher expression of Placenta-Expressed Transcript 1 Protein by immunofluorescent (FIG. 4c). These finding could also suggest a possible mechanism that could lead to the premature aging phenotype in ATR Seckel mice reported by Murga et al. In support of our finding, authors have reported accumulation of necrotic areas and overall loss of cellularity in mutant placentas that could also contribute to the dwarf phenotype regardless of intrinsic developmental defects.

### Example 6: ATR-Activated ESCs Gain Expanded Developmental Potential

Next, to understand if these cells have also gained distinct functional characteristics to give rise to extra-embryonic tissues along with inner cell mass derived lineages, FACS sorted positively pre-infected ESC with a lentivirus encoding mCherry protein were treated them with APH (FIG. 4e). Next, we injected five to eight cells into morula-stage embryos and 24 hrs post injection the contribution of cells into inner cell mass and trophectoderm were analyzed. Interestingly, in 7 out of 35 (20%) chimeric embryos, APH-induced 2C-like cells also contributed to the trophectoderm layer of the blastocysts, while none of the non-treated cells could contribute to the trophectoderm layer in CNTL condition (FIG. 4f). This result suggests that the developmental potential of ATR activated cells includes embryonic plus extra-embryonic tissues in contrast to ESC which are mostly restricted to generate embryonic cell types.

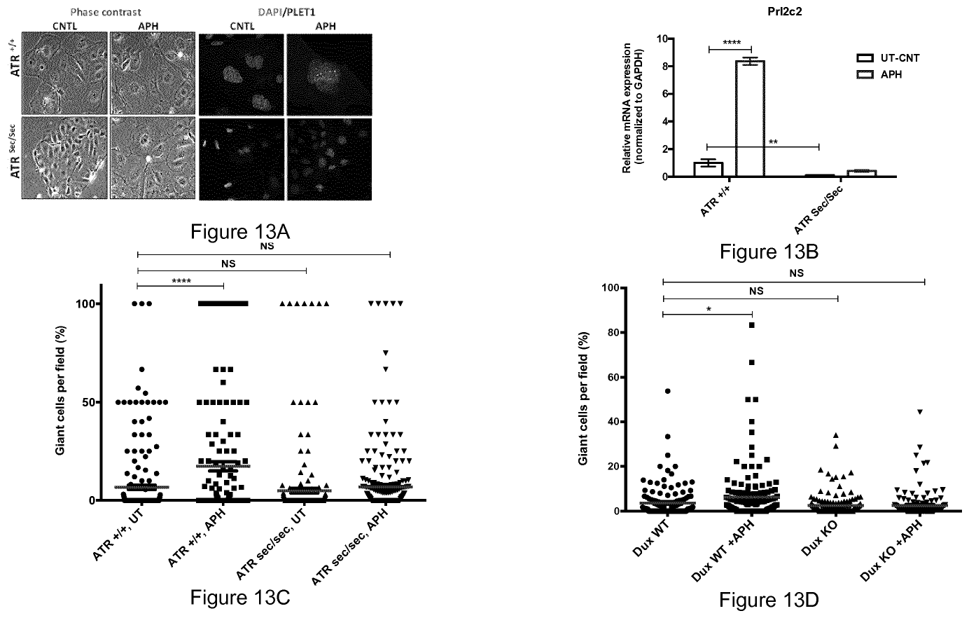

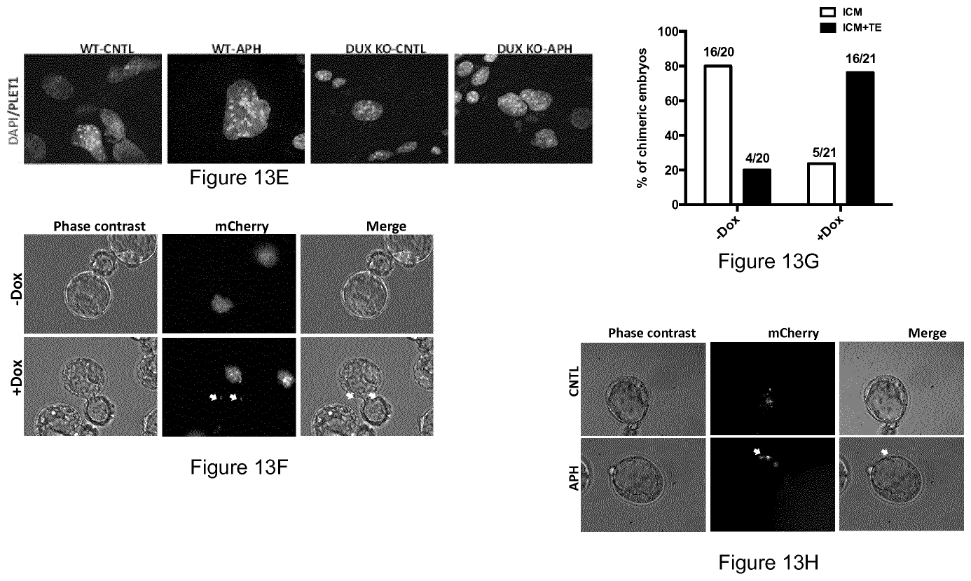

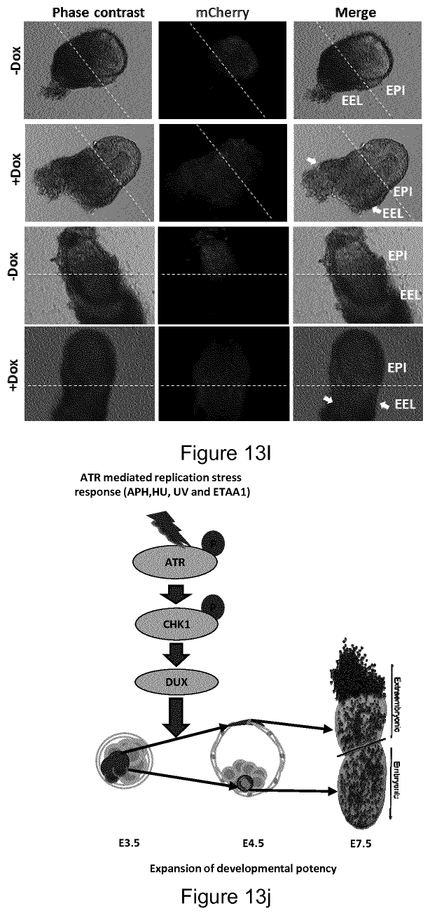

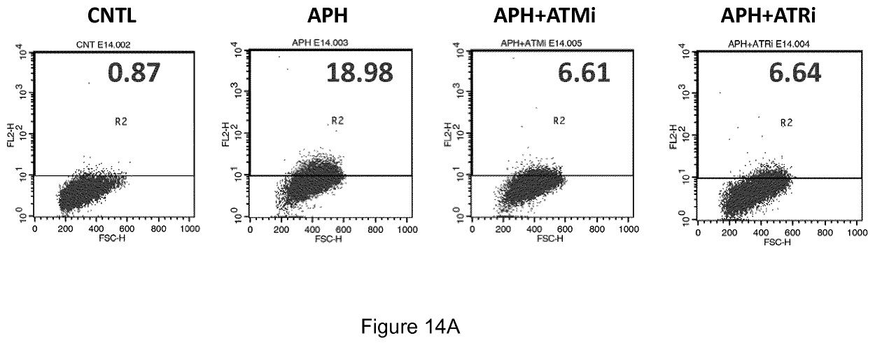

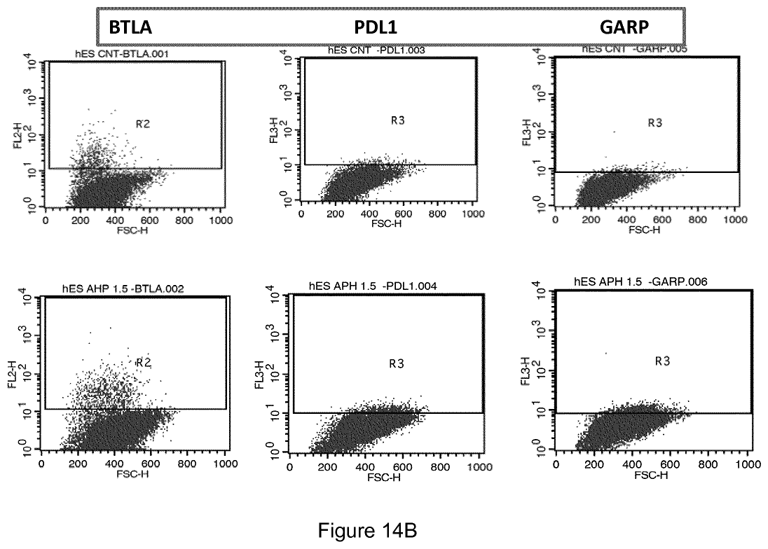

To test this hypothesis, both ATR^{Sec/Sec} and ATR^{+/+} ESCs were cultivated in the absence or in the presence of APH and were subsequently differentiated in vitro toward trophoblast-like stem cells (TSCs) for three days, followed by terminal differentiation to TGCs upon withdrawal of fibroblast growth factor 4 (FGF4) and heparin for three additional days (FIG. 13a)⁴⁰.

The qPCR results for the TGC specific marker, Pr/2c2, revealed the highest expression in ATR activated conditions, while ATR^{Sec/Sec} cells could not significantly upregulate this gene in the presence of APH (FIG. 13b). Moreover, Pr/2c2 basal level was four-fold higher in WT cells compared to non-treated ATR^{Sec/Sec} cells (FIG. 13b). These results were also consistent with the number of TGCs generated in each condition (FIG. 13c). Of note, TGCs had higher expression of Placenta-Expressed Transcript 1 protein (PLET1) as revealed by immunofluorescence (FIG. 13a).

Next, to understand whether ATR-induced differentiation to TGCs is also mediated through transition to 2C-like state, we differentiated Dux WT and KO ESCs toward trophoblast cells upon ATR activation (FIG. 13d,e). Significantly, ATR activation in DUX KO cells could not induce the formation of TGCs unlike WT cells, confirming the critical role of DUX in trophoblast-directed differentiation program (FIG. 13d). These experiments indicate that the transition to 2C-like cells is required for ATR-induced TGC differentiation.

Next, to define the cell fate potential of ATR-activated ESCs during embryonic development, we traced the fate of their progenies in chimeric blastocysts. To this end, ten mCherry fluorescent protein-labeled (mCherry-labeled) Dox-iETAA1 ESCs were treated with Dox and subsequently microinjected into each C57BL/6N recipient mouse morulae to generate chimeric blastocysts. Untreated Dox-iETAA1 ESCs were injected in parallel as control. Next, the contribution of ESCs to inner cell mass (ICM) and trophectoderm (TE) of the blastocysts was monitored 48 hrs post-injection (FIG. 13f). While all Dox-treated and untreated ESCs contributed to ICM, strikingly, ATR-activated ESCs progenies localized to both the ICM and TE layer of the blastocyst in approximately 76% of chimeras (FIG. 13g). Similar results were obtained when ATR was activated by APH treatment (FIG. 13h).

To further validate these findings, we then generated post-implantation chimeric embryos by microinjecting ten mCherry-labeled Dox-iETAA1 ESCs into each C57BL/6N morulae that were subsequently transferred to foster mothers. While WT ESCs contributed exclusively to the embryonic tissue (epiblast), ATR-activated cells contributed to both embryonic and extra-embryonic cell lineages in 50% (3 out of 6) of E7.5 (embryonic day 7.5) chimeric embryos (FIG. 13i). These results indicate that ATR-induced ESCs, gain expanded developmental potential (ATR-induced expanded potential stem cells, ATR-EPSc), in contrast to ESCs, which are mostly restricted to generate embryonic cell types (FIG. 13j).

### Example 7: ATR Induces Transcriptional Signature of 2C-Like Cells in ESCs Through DUX Activation

Next, to understand whether activation of ATR-dependent response could result in global transcriptional activation of 2C-like genes, we performed high-throughput transcriptional profiling on three ESC lines, namely E14, R1 and MC1 upon APH treatment. Analysis of differentially expressed genes (DEGs) (FDR<0.05 and |log 2 fold change|>1) identified 3074 upregulated genes with more than two-fold change in gene expression upon APH treatment, and only 640 downregulated genes (FIG. 11a), which is in agreement with general openness of the chromatin in 2C-like cells¹. To understand how many of the identified DEGs overlap with those specifically expressed in 2C-like cells, we compared our list of APH induced genes with a recently published dataset¹³. Through such comparison, we found that a significant fraction of APH induced retroviral elements and genes overlap with those expressed in Z⁺ population, including MERVL, MT2_Mm, Dux, Eif1a-like genes (Gm5662, Gm2022, Gm4027, and Gm8300), Zscan4 genes (Zscan4b and Zscan4d), Zfp352, Zfp750, Tdpoz genes (Tdpozl and Tdpoz3), and Tmem92 (FIG. 3b, c).

Considerably, qPCR results confirmed significant upregulation of main 2C-like genes upon APH treatment (FIG. 11d). Interestingly, the DEG analysis identified a large portion (48%) of APH-induced 2C specific genes to be transcriptionally suppressed upon ATR inhibition (FIG. 11e).

Recent reports demonstrated that 2C-like cells can be generated through genetic modulation of several factors including chromatin assembly factor-1 (CAF-1), KRAB (Kruppel-Associated Box Domain)-Associated Protein 1 (KAP1), microRNA-34 (miR-34) and DUX^{1,8,31,36}. Thus, to test whether these factors were involved in ATR-induced expression of 2C-like genes, we compared the transcriptome of APH-induced ESCs with those published datasets. While we found a significant overlap with all the datasets, the highest number of overlapping genes was found with the transcriptome of CAF-1 KD ESCs (FIG. 11f), possibly due to the previously reported role of CAF-1 in preventing RS³⁷. Next, we focused our analysis on the genes whose expression was reverted by ATRi to uncover a possible specific role of ATR in regulating these factors. Remarkably, through such analysis we found that 71% of the genes shared between APH and DUX-induced condition were rescued by ATRi (FIG. 11g), suggesting a possible role of DUX in activation of 2C-like genes through ATR. To validate this finding, we checked the expression of key 2C-like related genes in DUX KO ESCs after induction of RS.

Importantly, APH treatment could not induce the expression of key 2C-like genes in DUX KO ESCs, confirming that the expression of ATR-induced 2C-like genes requires DUX (FIG. 11h-j). These findings indicate that ATR-induced expression of main 2C-like genes is mediated by DUX, whose physiological role in the activation of cleavage-stage transcription program has been recently reported⁸⁻¹⁰.

### DISCUSSION

Here, we provide evidence that transition to bipotent 2C-like cells in ESCs culture is triggered by activation of ATR, a developmentally essential DDR gene, which plays a crucial role in the maintenance of stem cells both in embryonic and adult tissues^{35,41} First, we found a significant enrichment of γH2AX⁺ and CHK1+ cells within Z⁺ cell population. Second, inhibition of RSR by specific ATR and CHK1 inhibitors could markedly reduce the number of 2C-like cells in ESCs culture. Third, ATR-deficient Seckel and haploinsufficient CHK1 ESCs exposed to RS could not significantly induce 2C-like specific genes. Finally, robust activation of ATR through ETAA1 overexpression could further increase the bipotent 2C-like cell population in ESCs culture in the absence of RS.

Importantly, we unraveled the mechanistic basis of ATR-stimulated transition to bipotent 2C-like cells by showing that this response is mediated by DUX, the key inducer of zygotic genome activation (ZGA) in placental mammals^{10,39}. Although DNA damage induced cellular differentiation has been frequently shown in stem cells^{42,43}, here we report for first time to our knowledge that RS in ESC leads to the transition to a more developmentally potent state that is required for the subsequent differentiation toward TGCs. Our results show that, in response to RS, ATR triggers Dux expression that in turn increases bipotent 2C-like cells in ESC culture through global transcriptional activation of 2C-like genes, including genome caretaker genes Zscan4 and Tcstv1/3^{5,10,12,39}. In addition to their role in telomere elongation through activation of telomere sister chromatid exchange^{5,29}, Zscan4 genes could also promote DNA repair by facilitating heterochromatin decondensation and DNA demethylation^{13,30,44}. Therefore, ATR-mediated activation of Zscan4 genes likely contributes to promote ESCs genomic integrity in response to RS. Unexpectedly, our findings also show that ATR activation in ESCs can trigger the generation of ESCs with bidirectional cell fate potential (FIG. 13h). Activation of this pathway could act as a safeguard mechanism to ensure genome integrity of the developing embryo in response to RS by extending the contribution of ATR-activated ESCs to extra-embryonic tissues, thus limiting the incorporation of cells with unrepaired DNA in embryo proper tissues. However, how ATR coordinates genome stability maintenance through DNA repair and expansion of cell fate potential remains to be addressed.

ATR activation might also impact on cell fate at later stages of embryonic development. However, our findings clearly show that RS per se is not able to trigger 2C-like pathway in more committed embryonic cells such as MEFs, suggesting an involvement of robust repressive mechanisms in suppressing this pathway in differentiated cells. The consequence of unrepaired damage could be extremely serious during early embryonic development as it could subsequently result in embryonic lethality or transmission to the large populations of cells leading to teratogenicity or even germ lines incorporation. Therefore, it is vital for embryonic cells to respond efficiently to genotoxic stress through coordinated and integrated DNA damage repair pathways.

Though DNA damage induced cellular differentiation has been frequently reported in stem cells, to our knowledge, here for first time we report that replication stress in ESC, could lead to the transition to more primitive totipotent-like state followed by the global activation to 2C-like transcriptional network. More importantly, we found that the activation of 2C-like genes is mediated by ATR, which is reported to be essential to maintain stem cell self-renewal in embryonic and adult tissues. In addition to its involvement in DNA repair and response, ATR activation could boost the totipotency genes in face of exogenous and endogenous stresses. Upon failure of DNA repair, activation of trophectoderm differentiation program would prevent the contribution of ESCs with damaged DNA to developing tissues. It is tempting to speculate that activation of such ATR dependent pathway in committed adult stem cells might lead to expression of genes responsible for invasive and migratory behaviour of trophectoderm cells, which share key features with invasive and metastasizing cancer cells. This hypothesis would be compatible with replication stress being a major outcome of oncogene activity in cancer cells and would justify the potent suppressing role of ATR inhibitor in tumorigenesis.

In summary, our findings shed light on the endogenous and exogenous stimuli that could contribute to the cellular plasticity of ESCs and also provide a fundamental insight into the alternative mechanisms these cells exploit to respond to RS and maintain genome integrity.

### REFERENCES

1 Ishiuchi, T. et al. Early embryonic-like cells are induced by downregulating replication-dependent chromatin assembly. Nature Structural & Molecular Biology 22, 662-U635, doi:10.1038/nsmb.3066 (2015).
2 Macfarlan, T. S. et al. Embryonic stem cell potency fluctuates with endogenous retrovirus activity. Nature 487, 57-+, doi:10.1038/nature11244 (2012).
3 Zhou, L. Q. & Dean, J. Reprogramming the genome to totipotency in mouse embryos. Trends Cell Biol 25, 82-91, doi:10.1016/j.tcb.2014.09.006 (2015).
4 Akiyama, T. et al. Transient bursts of Zscan4 expression are accompanied by the rapid derepression of heterochromatin in mouse embryonic stem cells. DNA Res, doi:10.1093/dnares/dsv013 (2015).
5 Zalzman, M. et al. Zscan4 regulates telomere elongation and genomic stability in ES cells. Nature 464, 858-U866, doi:10.1038/nature08882 (2010).
6 Choi, Y. J. et al. Deficiency of microRNA miR-34a expands cell fate potential in pluripotent stem cells. Science 355, doi:10.1126/science.aag1927 (2017).
7 Amano, T. et al. Zscan4 restores the developmental potency of embryonic stem cells. Nature Communications 4, doi:Artn 1966 10.1038/Ncomms2966 (2013).
8 Hendrickson, P. G. et al. Conserved roles of mouse DUX and human DUX4 in activating cleavage-stage genes and MERVL/HERVL retrotransposons. Nature Genetics 49, 925-+, doi:10.1038/ng.3844 (2017).
9 Whiddon, J. L., Langford, A. T., Wong, C. J., Zhong, J. W. & Tapscott, S. J. Conservation and innovation in the DUX4-family gene network. Nature Genetics 49, 935-+, doi:10.1038/ng.3846 (2017).
De Iaco, A. et al. DUX-family transcription factors regulate zygotic genome activation in placental mammals. Nature Genetics 49, 941-+, doi:10.1038/ng.3858 (2017).
11 Amano, T. et al. Correction of Down syndrome and Edwards syndrome aneuploidies in human cell cultures. DNA Research 22, 331-342, doi:10.1093/dnares/dsv016 (2015).
12 Zhang, Q. et al. Tcstv1 and Tcstv3 elongate telomeres of mouse ES cells. Sci Rep 6, 19852, doi:10.1038/srep19852 (2016).
13 Eckersley-Maslin, M. A. et al. MERVL/Zscan4 Network Activation Results in Transient Genome-wide DNA Demethylation of mESCs. Cell Rep 17, 179-192, doi:10.1016/j.celrep.2016.08.087 (2016).
14 Falco, G. et al. Zscan4: A novel gene expressed exclusively in late 2-cell embryos and embryonic stem cells. Developmental Biology 307, 539-550, doi: 10.1016/j.ydbio.2007.05.003 (2007).
Jeong, Y. J. et al. Optimization of real time RT-PCR methods for the analysis of gene expression in mouse eggs and preimplantation embryos. Molecular Reproduction and Development 71, 284-289, doi: 10.1002/mrd.20269 (2005).
16 Bray, N. L., Pimentel, H., Melsted, P. & Pachter, L. Near-optimal probabilistic RNA-seq quantification. Nature Biotechnology 34, 525-527, doi:10.1038/nbt.3519 (2016).
17 Dobin, A. et al. STAR: ultrafast universal RNA-seq aligner. Bioinformatics 29, 15-21, doi:10.1093/bioinformatics/bts635 (2013).
18 Bolger, A. M., Lohse, M. & Usadel, B. Trimmomatic: a flexible trimmer for Illumina sequence data. Bioinformatics 30, 2114-2120, doi: 10.1093/bioinformatics/btu170 (2014).
19 Liao, Y., Smyth, G. K. & Shi, W. featureCounts: an efficient general purpose program for assigning sequence reads to genomic features. Bioinformatics 30, 923-930, doi: 10.1093/bioinformatics/btt656 (2014).
Soneson, C., Love, M. I. & Robinson, M. D. Differential analyses for RNA-seq: transcript-level estimates improve gene-level inferences. F1000Res 4, 1521, doi: 10.12688/f1000research.7563.2 (2015).
21 Ritchie, M. E. et al. limma powers differential expression analyses for RNA-sequencing and microarray studies. Nucleic Acids Res 43, e47, doi:10.1093/nar/gkv007 (2015).
22 Law, C. W., Chen, Y. S., Shi, W. & Smyth, G. K. voom: precision weights unlock linear model analysis tools for RNA-seq read counts. Genome Biology 15, doi:ARTN R29 10.1186/gb-2014-15-2-r29 (2014).
23 McCarthy, D. J., Chen, Y. S. & Smyth, G. K. Differential expression analysis of multifactor RNA-Seq experiments with respect to biological variation. Nucleic Acids Research 40, 4288-4297, doi:10.1093/nar/gks042 (2012).
24 Wu, D. & Smyth, G. K. Camera: a competitive gene set test accounting for inter-gene correlation. Nucleic Acids Res 40, e133, doi:10.1093/nar/gks461 (2012).
Hanzelmann, S., Castelo, R. & Guinney, J. GSVA: gene set variation analysis for microarray and RNA-Seq data. Bmc Bioinformatics 14, doi:Artn 7 10.1186/1471-2105-14-7 (2013).
26 Giachino, C., Orlando, L. & Turinetto, V. Maintenance of Genomic Stability in Mouse Embryonic Stem Cells: Relevance in Aging and Disease. International Journal of Molecular Sciences 14, 2617-2636, doi:10.3390/ijms14022617 (2013).
27 Macosko, E. Z. et al. Highly Parallel Genome-wide Expression Profiling of Individual Cells Using Nanoliter Droplets. Cell 161, 1202-1214, doi:10.1016/j.cell.2015.05.002 (2015).
28 Aze, A., Sannino, V., Soffientini, P., Bachi, A. & Costanzo, V. Centromeric DNA replication reconstitution reveals DNA loops and ATR checkpoint suppression. Nature Cell Biology 18, 684-+, doi:10.1038/ncb3344 (2016).
29 Nakai-Futatsugi, Y. & Niwa, H. Zscan4 Is Activated after Telomere Shortening in Mouse Embryonic Stem Cells. Stem Cell Reports 6, 483-495, doi:10.1016/j.stemcr.2016.02.010 (2016).
30 Akiyama, T. et al. Transient bursts of Zscan4 expression are accompanied by the rapid derepression of heterochromatin in mouse embryonic stem cells. DNA Res 22, 307-318, doi:10.1093/dnares/dsv013 (2015).
31 Choi, Y. J. et al. Deficiency of microRNA miR-34a expands cell fate potential in pluripotent stem cells. Science 355, 596-+, doi:10.1126/science.aag1927 (2017).
32 Storm, M. P. et al. Zscan4 Is Regulated by PI3-Kinase and DNA-Damaging Agents and Directly Interacts with the Transcriptional Repressors LSD1 and CtBP2 in Mouse Embryonic Stem Cells. Plos One 9, doi:ARTN e89821 10.1371/journal.pone.0089821 (2014).
33 Murga, M. et al. A mouse model of ATR-Seckel shows embryonic replicative stress and accelerated aging. Nature Genetics 41, 891-U846, doi:10.1038/ng.420 (2009).
34 Liu, Q. H. et al. Chk1 is an essential kinase that is regulated by Atr and required for the G(2)/M DNA damage checkpoint. Genes & Development 14, 1448-1459 (2000).
35 Brown, E. J. & Baltimore, D. ATR disruption leads to chromosomal fragmentation and early embryonic lethality. Genes & Development 14, 397-402 (2000).
36 Rowe, H. M. et al. KAP1 controls endogenous retroviruses in embryonic stem cells. Nature 463, 237-240, doi: 10.1038/nature08674 (2010).
37 Hoek, M. & Stillman, B. Chromatin assembly factor 1 is essential and couples chromatin assembly to DNA replication in vivo. Proceedings of the National Academy of Sciences of the United States of America 100, 12183-12188, doi:10.1073/pnas.1635158100 (2003).
38 Bass, T. E. et al. ETAA1 acts at stalled replication forks to maintain genome integrity. Nature Cell Biology 18, 1185-+, doi:10.1038/ncb3415 (2016).
39 Haahr, P. et al. Activation of the ATR kinase by the RPA-binding protein ETAA1. Nat Cell Biol 18, 1196-1207, doi:10.1038/ncb3422 (2016).
Abad, M. et al. Reprogramming in vivo produces teratomas and iPS cells with totipotency features. Nature 502, 340-+, doi:10.1038/nature12586 (2013).
41 Ruzankina, Y. et al. Deletion of the developmentally essential gene ATR in adult mice leads to age-related phenotypes and stem cell loss. Cell Stem Cell 1, 113-126, doi:10.1016/j.stem.2007.03.002 (2007).
42 Schneider, L. et al. DNA Damage in Mammalian Neural Stem Cells Leads to Astrocytic Differentiation Mediated by BMP2 Signaling through JAK-STAT. Stem Cell Reports 1, 123-138, doi:10.1016/j.stemcr.2013.06.004 (2013).
43 Santos, M. A. et al. DNA-damage-induced differentiation of leukaemic cells as an anti-cancer barrier. Nature 514, 107-+, doi:10.1038/nature13483 (2014).
44 Dan, J. M. et al. Zscan4 Inhibits Maintenance DNA Methylation to Facilitate Telomere Elongation in Mouse Embryonic Stem Cells. Cell Reports 20, 1936-1949, doi:10.1016/j.celrep.2017.07.070 (2017).
Ferretti, C., Bruni, L., Dangles-Marie, V., Pecking, A. P. & Bellet, D. Molecular circuits shared by placental and cancer cells, and their implications in the proliferative, invasive and migratory capacities of trophoblasts. Human Reproduction Update 13, 121-141, doi:10.1093/humupd/dm1048 (2007).
46 Yasuda, T. et al. Recurrent DUX4 fusions in B cell acute lymphoblastic leukemia of adolescents and young adults (vol 48, pg 569, 2016). Nature Genetics 48, 1591-1591 (2016).
47 Yoshimoto, T. et al. CIC-DUX4 Induces Small Round Cell Sarcomas Distinct from Ewing Sarcoma. Cancer Research 77, 2927-2937, doi: 10.1158/0008-5472.Can-16-3351 (2017).
48 Macheret, M. & Halazonetis, T. D. DNA Replication Stress as a Hallmark of Cancer. Annual Review of Pathology: Mechanisms of Disease, Vol 10 10, 425-448, doi:10.1146/annurev-pathol-012414-040424 (2015).
49 Dobbelstein, M. & Sorensen, C. S. Exploiting replicative stress to treat cancer. Nature Reviews Drug Discovery 14, 405-423, doi:10.1038/nrd4553 (2015).
50 Nieto-Soler, M. et al. Efficacy of ATR inhibitors as single agents in Ewing sarcoma. Oncotarget 7, 58759-58767, doi:10.18632/oncotarget.11643 (2016).
51 Bartkova, J. et al. DNA damage response as a candidate anti-cancer barrier in early human tumorigenesis. Nature 434, 864-870, doi:10.1038/nature03482 (2005).
52 Bartek, J., Mistrik, M. & Bartkova, J. Thresholds of replication stress signaling in cancer development and treatment. Nat Struct Mol Biol 19, 5-7, doi:10.1038/nsmb.2220 (2012).
53 Smith, Z. D. et al. Epigenetic restriction of extraembryonic lineages mirrors the somatic transition to cancer. Nature 549, 543-547, doi:10.1038/nature23891 (2017).

1. A method for inducing the expression of one or more 2 cell-like genes in a pluripotent stem cell, the method comprising activating replication stress response in a pluripotent stem cell.

2. The method of claim 1, comprising inducing replication stress in the pluripotent stem cell.

3. The method of claim 2, comprising exposing the pluripotent stem cell to one or more DNA damage inducing agents.

4. The method of claim 1, comprising culturing the pluripotent stem cell in the presence of one or more agent selected from Aphidicolin, ATR, ETAA1, hydroxyurea or high O₂ or exposing the cell to UV radiation or ionising radiation.

5. A method for inducing the expression of one or more 2 cell-like genes in a pluripotent stem cell, the method comprising activating the ATR pathway in the pluripotent stem cell.

6. The method of claim 1 or claim 5, comprising overexpressing ETAA1 or an ATR binding fragment thereof in the pluripotent stem cell.

7. The method of claim 6, wherein the pluripotent stem cell is infected with a lentivirus that expresses ETAA1 or an ATR binding fragment thereof.

8. The method of any one of the preceding claims wherein the one or more 2 cell-like genes is selected from the group comprising Eif1a-like genes (Gm5662, Gm2022, Gm4027, Gm2016, and Gm8300), MERVL, MT2_Mm, Dux, Gm4981, Zfp352, Zfp750, Tdpoz genes (Tdpozl and Tdpoz3), and Tmem92, Zscan4 genes (Zscan4a-Zscan4d), Dux, Tcstv3, GM12794, γH2AX, P-CHK1, GM4340, Gm12794, Afp352, Sfp750, Tdpoz, Eif1a and Tmem19.

9. The method of any one of the preceding claims wherein the cell expressing one or more 2 cell-like genes is substantially totipotent.

10. The method of any one of the preceding claims wherein the pluripotent stem cell is an induced pluripotent stem cell or an embryonic stem cell.

11. The method of any one of the preceding claims, further comprising a step of differentiating the cell into an embryonic or extra-embryonic tissue cell.

12. The method of any one of the preceding claims, further comprising isolating a cell expressing one or more 2 cell-like genes from pluripotent cells that do not express one or more 2 cell-like genes.

13. A 2 cell-like stem cell prepared by the method of any one of claims 1 to 12.

14. A stem cell that expresses one or more of Zscan4 genes, Tcstv3, GM12794, Eif, muERV-L, γH2AX, P-CHK1, GM4340, Gm12794, Afp352, Sfp750, Tdpoz, Eif1a and Tmem19.

15. Stem cell culture medium comprising a DNA damage inducing agent, the DNA damage agent being optionally selected from Aphidicolin, hydroxyurea or high O₂.

16. Use of a DNA damage inducing agent in the culture of stem cells.

17. Stem cell culture medium comprising an ATR activating agent.

18. Use of an ATR activating agent in the culture of stem cells.

19. A population of pluripotent stem cells wherein at least 6%, 7%, 8%, 9% or 10% of the stem cells express one or more 2C like genes.

20. A pluripotent stem cell containing a plasmid encoding ETAA1 or an ATR fragment thereof.
